(19) United States
(12) Patent Application Publication
(10) Pub. No.: US 2013/0158031 A1
(43) Pub. Date: Jun. 20, 2013

(54) ALKYNE-BRIDGED HETERO-AROMATICS AND USES THEREOF

(71) Applicants: Zhen-Wei CAI, Belle Mead, NJ (US); Ding ZHOU, Shanghai (CN); Yougang LIN, Shanghai (CN); Ping CHEN, Hillsborough, NJ (US)

(72) Inventors: Zhen-Wei CAI, Belle Mead, NJ (US); Ding ZHOU, Shanghai (CN); Yougang LIN, Shanghai (CN); Ping CHEN, Hillsborough, NJ (US)

(73) Assignee: SU ZHOU JING HONG BIOTECH CO., LTD., Shanghai (CN)

(21) Appl. No.: 13/718,113

(22) Filed: Dec. 18, 2012

Related U.S. Application Data
(60) Provisional application No. 61/591,021, filed on Jan. 26, 2012.

(30) Foreign Application Priority Data
Dec. 19, 2011 (CN) 201110428284.0

(51) Int. Cl. C07D 401/14; C07D 401/10

(52) U.S. Cl. CPC C07D 401/14; C07D 401/10
USPC 514/235.2; 546/176; 514/314; 544/284; 514/266.21; 546/256; 514/333; 544/333; 514/256; 544/128

(57) ABSTRACT

This invention relates to novel alkyne-bridged hetero-aromatics as described in the specification which are PDE10A inhibitors and useful for the treatment of neurological, psychiatric disorder, metabolic disorders, such as Schizophrenia, Parkinson's disease, Huntington's disease, Alzheimer's disease, learning memory disorder, drug addiction (abuse), sleeping disorder, depression, obesity, non-insulin dependent diabetes, bipolar disorder, obsessive compulsive disorder, or pain; to process for their preparation; to pharmaceutical compositions comprising them; and to methods of using them.

### CROSS REFERENCE TO RELATED APPLICATIONS

This application claims the benefit of Chinese Application No. 201110428284.0, filed Dec. 19, 2011, and U.S. Provisional Patent Application No. 61/591,021, filed Jan. 26, 2012, the entire contents of which are hereby incorporated by reference herein.

### FIELD OF THE INVENTION

This invention relates to novel alkyne-bridged hetero-aromatics which are PDE10A inhibitors and useful for the treatment of neurological, psychiatric disorder, metabolic disorders, such as Schizophrenia, Parkinson's disease, Huntington's disease, Alzheimer's disease, learning memory disorder, drug addiction (abuse), sleeping disorder, depression, obesity, non-insulin dependent diabetes, bipolar disorder, obsessive compulsive disorder, or pain; to process for their preparation; to pharmaceutical compositions comprising them; and to methods of using them.

### BACKGROUND OF THE INVENTION

Cyclic nucleotide phosphodiesterases (PDEs) are enzymes that catalyze the hydrolytic degradation of cAMP and cGMP and thus are the key regulators of intracellular cyclic nucleotide levels. cAMP and cGMP serve as important second messengers in the signal cascade of G-protein coupled receptors and mediate the biological response of a variety of extracellular signals like hormones, light, and neurotransmitters. Up to date, the PDEs are discovered and divide into 11 families with are encoded by 21 genes. Phosphodiesterase PDE10A is a ˜89 kD PDE family member expressed primarily in brain and testes and is highly localized in GABAergic medium spiny neurons in the striatum. This localization of PDE10A is expected to have an influence on the dopaminergic and glutamatergic pathways both which play critical roles in the pathology of several psychotic and neurodegenerative disorders. The human PDE10A sequence is reported to have high homology to both rat and mouse with 95% amino acid identity and 98% identity conserved in the catalytic domain.

PDE10A has the capability to hydrolyze both cAMP and cGMP, having a higher affinity for cAMP (Km=0.05 uM) than cGMP (Km=3 uM) (Soderling et. al. Proc. Natl. Sci. USA, 1999, 96(12), 7071-7076). Inhibition of PDE10A induces increase of cAMP and cGMP and regulates their production from these brain regions, all of which have been implicated in schizophrenia that is a severe and chronic mental illness affecting nearly 1% worldwide populations. The symptoms of schizophrenia includes hallucinations and delusions, or at the other extreme, anhedonia or social withdrawal. All these symptoms are indicative of cognitive impairment and functional disability. By inhibiting PDE10A activity, levels of cAMP and cGMP are increased within neurons, and the ability of these neurons to function properly is thereby improved. Therefore, PDE10A inhibitors are being developed in the treatment of schizophrenia and additionally, a variety of conditions as described herein like Parkinson's disease, Huntington's disease, Alzheimer's disease, learning memory disorder, drug addiction (abuse), sleeping disorder, and depression. They can also be used in treatment of other conditions or disorders such as obesity, non-insulin dependent diabetes, bipolar disorder, obsessive compulsive disorder, and pain. Mechanistically, PDE10A inhibition by upregulating cAMP and cGMP levels mimics the effects of D2 dopamine receptor antagonism that is the standard treatment for psychosis, along with D1 agonism which may lead to the minimization of the side-effect liabilities while contributing to a pro-cognitive profile.

### SUMMARY OF THE INVENTION

In one aspect, the present invention provides a compound having the following Formulae, or a pharmaceutical acceptable salt thereof:

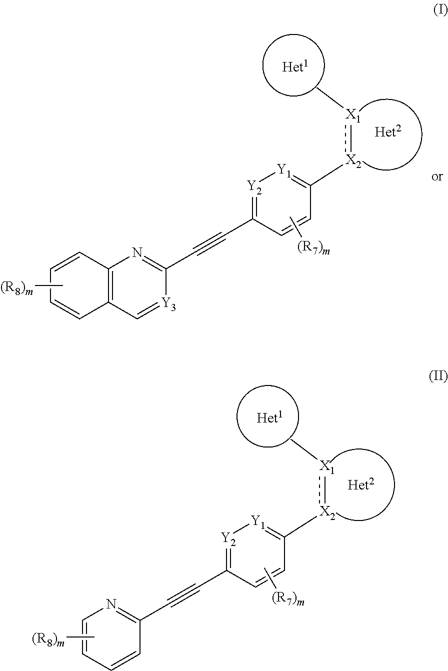

wherein X₁ and X₂ are each independently C or N;
Y₁, Y₂ and Y₃ are each independently C or N;
each Het¹ is independently aryl or 3-7-membered heterocycle containing at least one heteroatom selected from the group consisting of nitrogen, oxygen, and sulfur, in which said aryl may be optionally substituted by one to three R₁ and one R₂, and said heterocycle may be optionally substituted by one to three R₁;
each Het² is independently 3-7-membered heterocycle containing at least one heteroatom selected from the group consisting of nitrogen, oxygen, and sulfur, in which said heterocycle may be optionally substituted by one R₅ and one to three R₆;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₂ is independently H, OH, CN, or NRₐR_{b};
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In another aspect, the present invention provides a pharmaceutical composition comprising at least one compound as described herein and a pharmaceutically-acceptable carrier or diluent.

In yet another aspect, the present invention provides a method for treating a psychotic disorder in a mammalian species in need thereof, the method comprising administering to the mammalian species a therapeutically effective amount of at least one compound as described herein, wherein the psychotic disorder is selected from schizophrenia, delusional disorders and drug induced psychosis.

In yet another aspect, the present invention provides a method for treating an anxiety disorder in a mammalian species in need thereof, the method comprising administering to the mammalian species a therapeutically effective amount of at least one compound as described herein, wherein the anxiety disorder is selected from panic disorder, agoraphobia, a specific phobia, social phobia, obsessive-compulsive disorder, post-traumatic stress disorder, acute stress disorder, and generalized anxiety disorder.

In a further another aspect, the present invention provides a method for treating a neurodegenerative disorder in a mammalian species in need thereof, the method comprising administering to the mammalian species a therapeutically effective amount of at least one compound as described herein, wherein the neurodegenerative disorder is selected from Parkinson's disease, Huntington's disease, dementia, Alzheimer's disease, multi-infarct dementia, AIDS-related dementia, Fronto temperal Dementia, neurodegeneration associated with cerebral trauma, neurodegeneration associated with stroke, neurodegeneration associated with cerebral infarct, hypoglycemia-induced neurodegeneration, neurodegeneration associated with epileptic seizure, neurodegeneration associated with neurotoxin poisoning, and multi-system atrophy.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. The term “(C₁-C₆)alkyl” refers to a straight or branched chain alkane (hydrocarbon) radical containing from 1 to 6 carbon atoms, such as n-hexyl, 2-methylpentyl, 3-methylpentyl, 4-methylpentyl, 2,3-dimethylbutyl, 2,2-dimethylbutyl, in addition to those exemplified for “(C₁-C₄)alkyl.” “Substituted alkyl” refers to an alkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c}, together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substitutents, groups such as alkyl, cycloalkyl, alkenyl, alkynyl, cycloalkenyl, heterocycle and aryl can themselves be optionally substituted.

The term “alkenyl” refers to a straight or branched chain hydrocarbon radical containing from 2 to 12 carbon atoms and at least one carbon-carbon double bond. Exemplaries of such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethyl-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted.

The term “alkynyl” refers to a straight or branched chain hydrocarbon radical containing from 2 to 12 carbon atoms and at least one carbon to carbon triple bond. An exemplary of such groups includes ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, hex-3-ynyl. “Substituted alkynyl” refers to an alkynyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substitutents can themselves be optionally substituted.

The term “cycloalkenyl” refers to a partially unsaturated cyclic hydrocarbon group containing 1 to 4 rings and 3 to 8 carbons per ring. Exemplaries of such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). “Substituted aryl” refers to an aryl group substituted by one or more substituents, preferably 1 to 3 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl;

each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “alkylamino” refers to a group having the structure —NHR′, wherein R′ is hydrogen, alkyl or substituted alkyl, cycloalkyl or substituted cycloalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cyclolalkenyl, aryl or substituted aryl, heterocylyl or substituted heterocyclyl, as defined herein. R and R′ may be the same or different in an dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

The terms “halogen” or “halo” refer to chlorine, bromine, fluorine or iodine.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of a compound of the present invention may be formed, for example, by reacting a compound I with an amount of acid or base, such as an equivalent amount, in a medium such as one in which the salt precipitates or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

Compounds of the present invention which contain an acidic moiety, such but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to greater than 95%, equal to or greater than 99% pure (“substantially pure” compound I), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specifications, groups and substituents thereof may be chosen to provide stable moieties and compounds.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, or an enantiomer, diastereomer, tautomer, or pharmaceutically acceptable salt or solvate thereof, which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds can generally be prepared by carrying out the procedures disclosed in the Schemes and/or in the Examples below, by substituting a readily available isotopically labeled reagent for a non-isotopically labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

### Compounds

The novel alkyne-bridged hetero-aromatics compounds of the present invention are PDE10A inhibitors.

In one aspect, the present invention provides a compound having the following Formulae, or a pharmaceutical acceptable salt thereof:

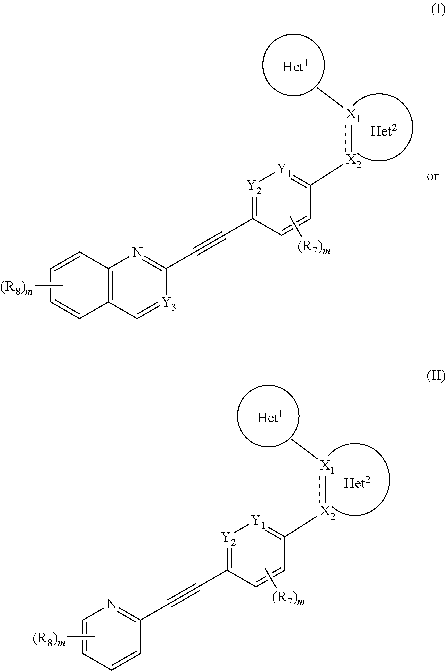

wherein X₁ and X₂ are each independently C or N;
Y₁, Y₂ and Y₃ are each independently C or N;
each Het¹ is independently aryl or 3-7-membered heterocycle containing at least one heteroatom selected from the group consisting of nitrogen, oxygen, and sulfur, in which said aryl may be optionally substituted by one to three R₁ and one R₂, and said heterocycle may be optionally substituted by one to three R₁;
each Het² is independently 3-7-membered heterocycle containing at least one heteroatom selected from the group consisting of nitrogen, oxygen, and sulfur, in which said heterocycle may be optionally substituted by one R₅ and one to three R₆;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₂ is independently H, OH, CN, or NRₐR_{b};
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In certain embodiments, the present invention provides a compound of Formula (I):

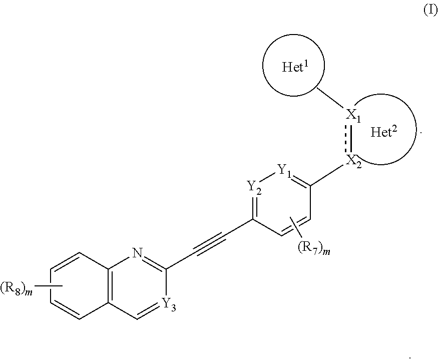

In certain other embodiments, the present invention provides a compound of Formula (II):

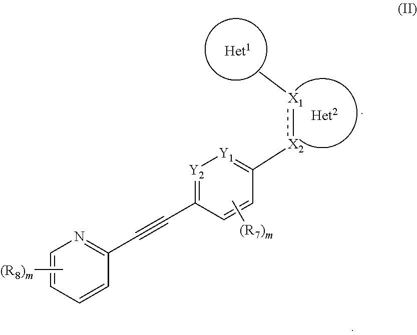

In certain embodiments, Het¹ is independently selected from Formulae A1-A5 below:

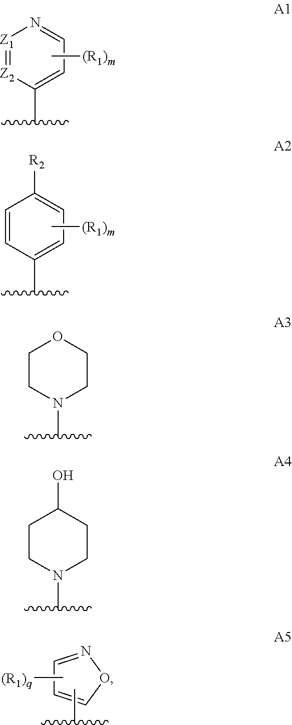

Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₂ is independently H, OH, CN, or NRₐR_{b};
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
m is 1-3; and
q is 1-2.

In certain embodiments, each Het² is independently selected from Formulae B1-B23 below:

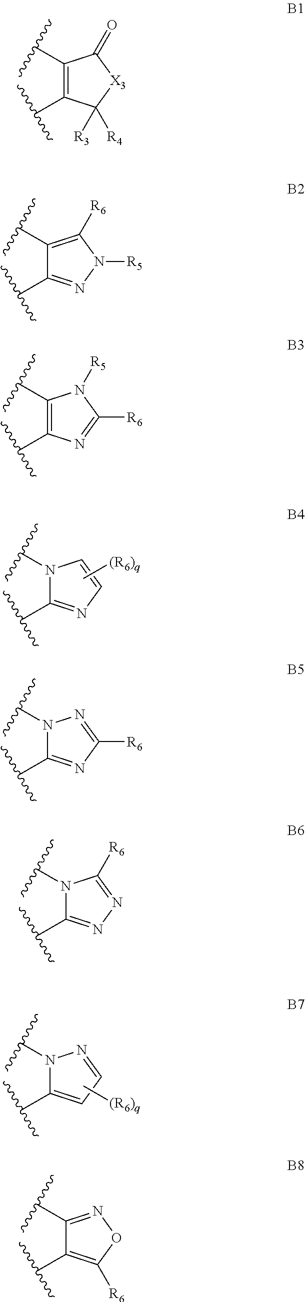

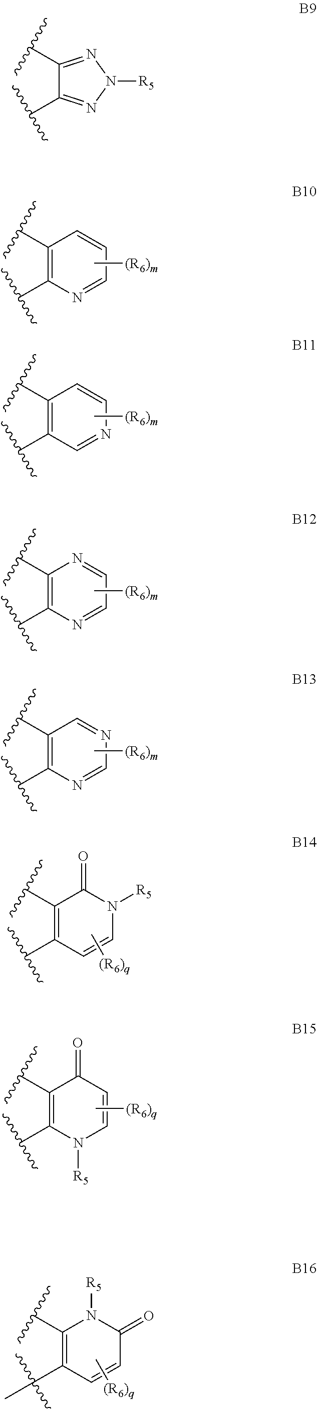

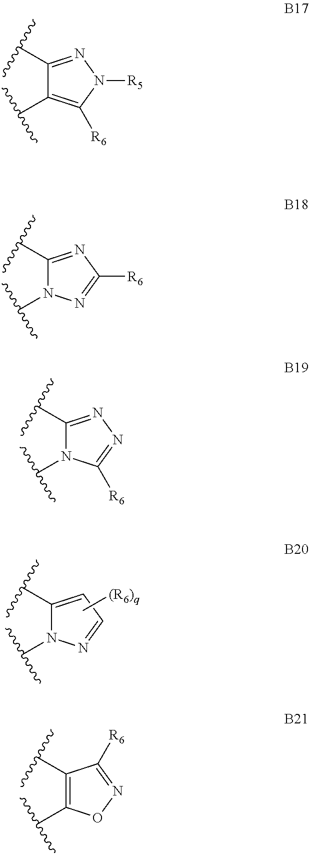

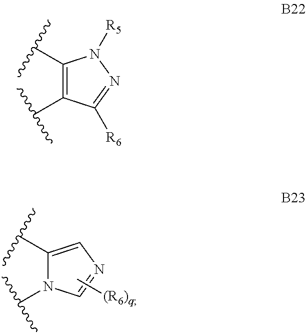

wherein X₃ is O, or NR₅;
each R₃ and R₄ is independently H, or (C₁-C₆)alkyl;
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl; each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
m is 1-3; and
q is 1-2.

In certain embodiments, the present invention provides a compound of Formula (III):

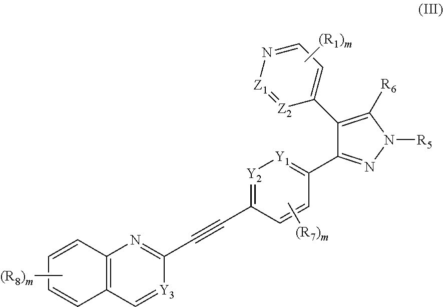

wherein Y₁, Y₂ and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In certain embodiments, the present invention provides a compound of Formula (IIIa):

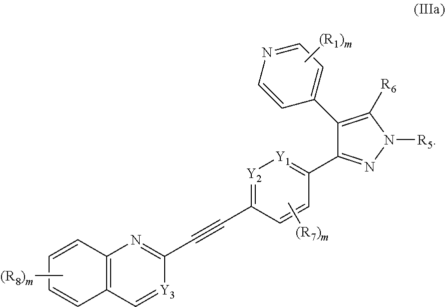

In certain embodiments, the present invention provides a compound of Formula (IIIc):

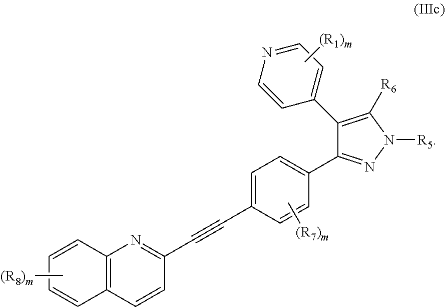

In certain embodiments, the present invention provides a compound of Formula (IIId):

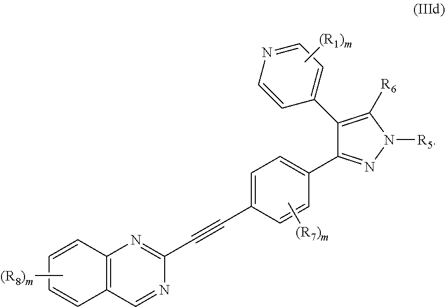

In certain embodiments, the present invention provides a compound of Formula (IV):

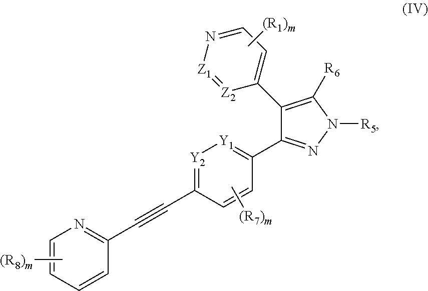

wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In certain embodiments, the present invention provides a compound of Formula (IVa):

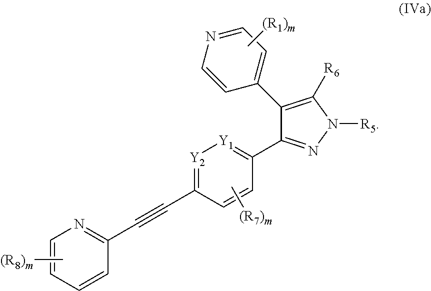

In certain embodiments, the present invention provides a compound of Formula (IVb):

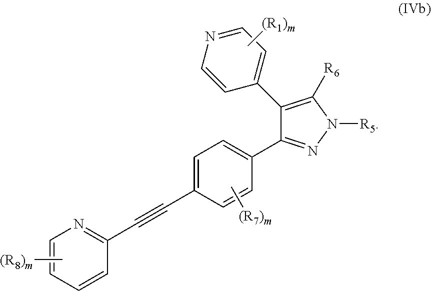

In certain embodiments, the present invention provides a compound of Formula (V):

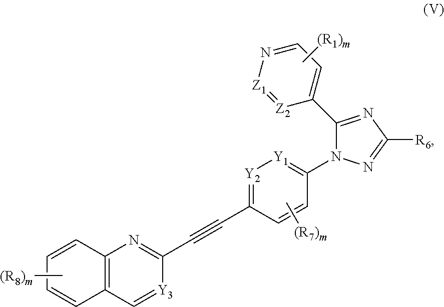

wherein Y₁, Y₂ and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In certain embodiments, the present invention provides a compound of Formula (Va):

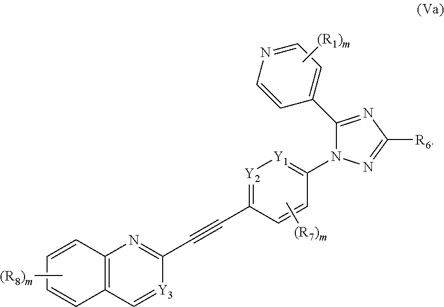

In certain embodiments, the present invention provides a compound of Formula (Vb):

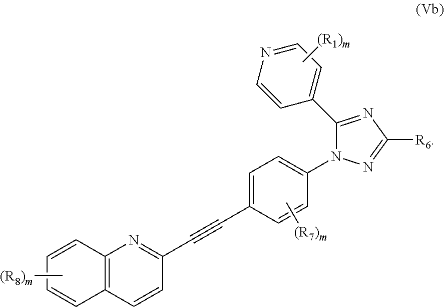

In certain embodiments, the present invention provides a compound of Formula (VI):

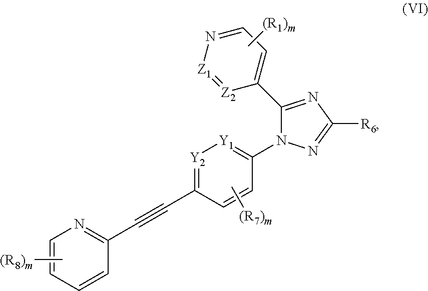

wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In certain embodiments, the compound of Formula (VI) has the structure of Formula (VIa):

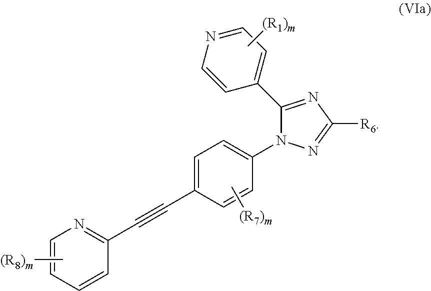

In certain embodiments, the present invention provides a compound of Formula (VII):

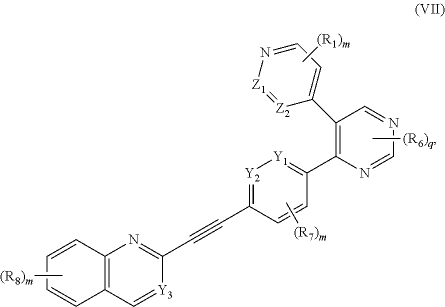

wherein Y₁, Y₂ and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6;
m is 1-3; and
q is 1-2.

In certain embodiments, the present invention provides a compound of Formula (VIII):

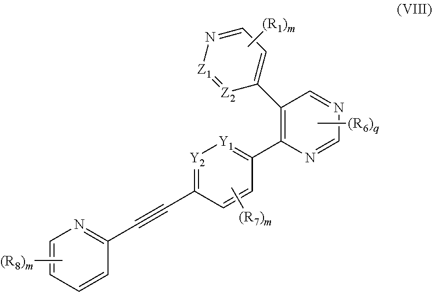

wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In certain embodiments, the present invention provides a compound of Formula (IX):

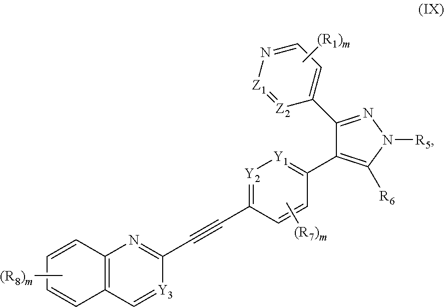

wherein Y₁, Y₂, and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
m is 1-3.

In certain embodiments, the present invention provides a compound of Formula (X):

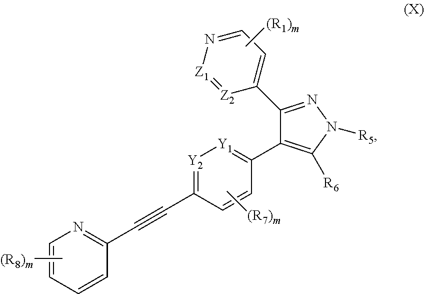

wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In certain embodiments, the present invention provides a compound of Formula (XI):

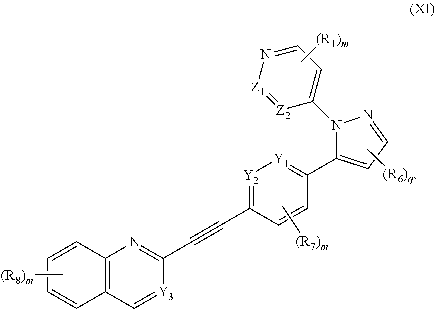

wherein Y₁, Y₂, and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
q is 1-2; and m is 1-3.

In certain embodiments, the present invention provides a compound of Formula (XII):

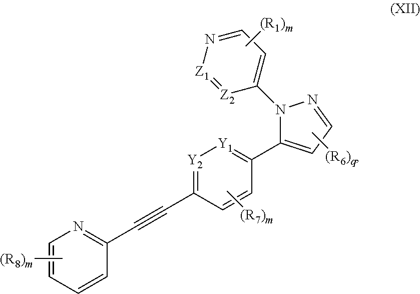

wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In certain embodiments, the present invention provides a compound of Formula (XIII):

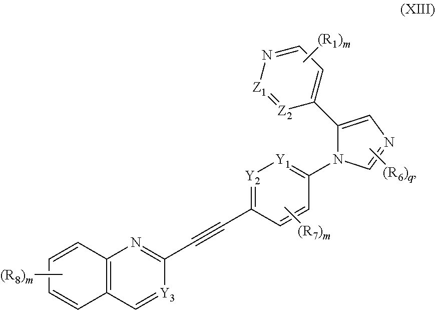

wherein Y₁, Y₂, and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
q is 1-2; and m is 1-3.

In certain embodiments, the present invention provides a compound of Formula (XIV):

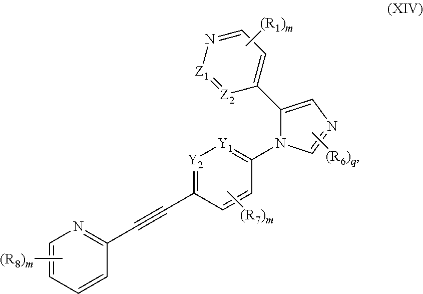

wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In certain embodiments, the present invention provides a compound of Formula (XV):

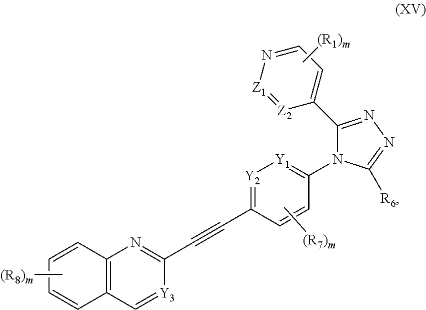

wherein Y₁, Y₂, and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
q is 1-2; and m is 1-3.

In certain embodiments, the present invention provides a compound of Formula (XVI):

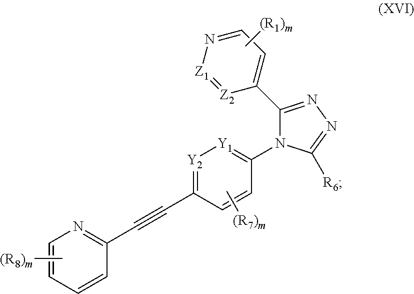

wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In certain embodiments, the present invention provides a compound of Formula (XVII):

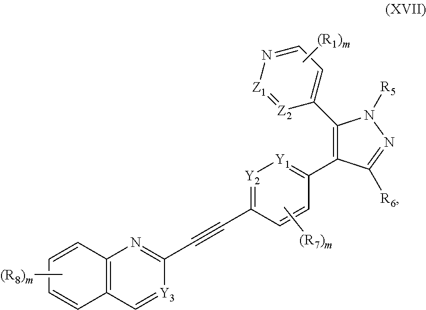

wherein Y₁, Y₂, and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃); each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
q is 1-2; and m is 1-3.

In certain embodiments, the present invention provides a compound of Formula (XVIII):

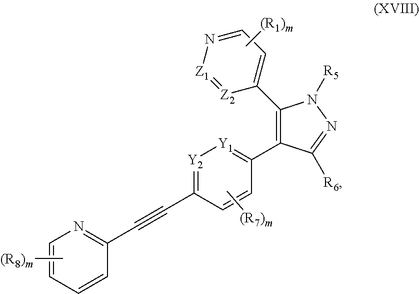

wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In certain embodiments, the present invention provides a compound of Formula (IXX):

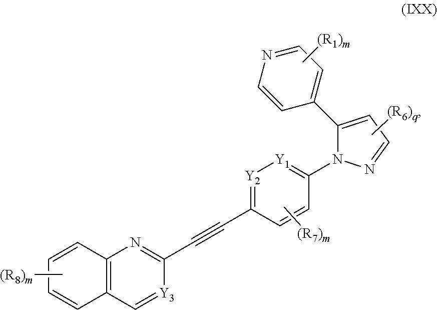

wherein Y₁, Y₂, and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
q is 1-2; and m is 1-3.

In certain embodiments, the present invention provides a compound of Formula (XX):

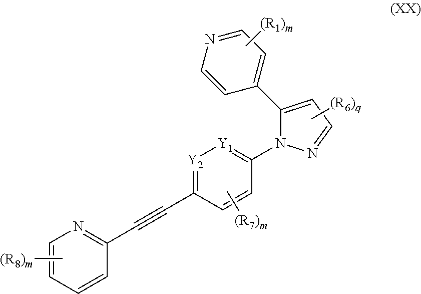

wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

In certain embodiments, Z₁ and Z₂ are each independently C.

In certain embodiments, each Y₃ is independently C.

In certain embodiments, each Y₃ is independently N.

In certain embodiments, Y₁ and Y₂ are each independently C. In certain other embodiments, one of Y₁ and Y₂ is independently C, and the other of Y₁ and Y₂ is independently N.

In certain embodiments, each R₁ is independently H. In certain other embodiments,

each R₁ is independently (C₁-C₄)alkyl and m is 1.

In certain embodiments, each R₅ is independently H, (C₁-C₄)alkyl, or (C₃-C₇)cycloalkyl. In certain other embodiments, each R₅ is independently (CH₂)ₙOH, or (CH₂)ₙ(C₁-C₄)alkoxy, and n is 1-6.

In certain embodiments, each R₅ is independently (CH₂)ₙNRₐR_{b}, in which n is 1-6, and each occurrence of Rₐ and R_{b} is independently hydrogen or (C₁-C₆)alkyl, or Rₐ and R_{b}, together with the nitrogen atom to which they are attached, form a ring selected from:

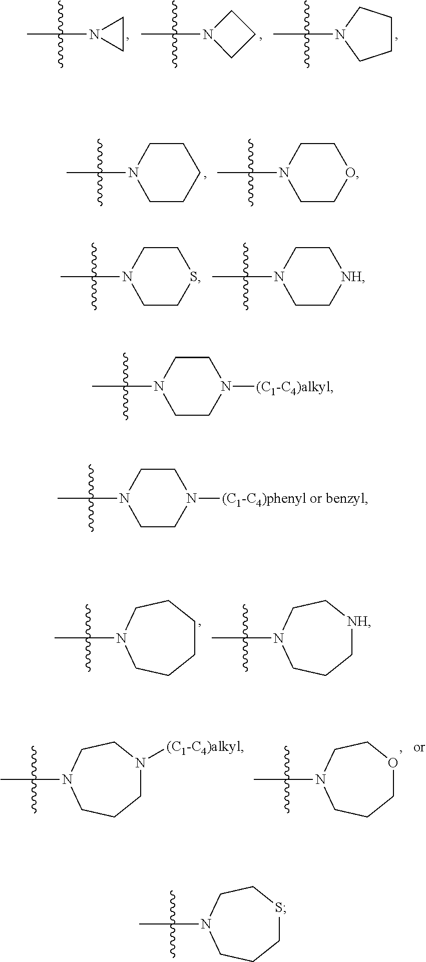

and n is 1-6.

In certain embodiments, each R₆ is independently H, halogen, OH, CN, (C₁-C₆)alkyl, (C₁-C₆)alkoxy, or (C₁-C₆)haloalkyl (e.g., CF₃). In certain other embodiments, each R₆ is independently H.

In certain embodiments, each R₇ is independently H, halogen, OH, CN, OCF₃, (C₁-C₆)alkyl, (C₁-C₆)alkoxy, or (C₁-C₆)haloalkyl (e.g., CF₃), and m is 1. In certain other embodiments, each R₇ is independently H.

In certain embodiments, each R₈ is independently H, halogen, OH, CN, (C₁-C₆)alkyl, (C₁-C₆)alkoxy, (C₃-C₇)cycloalkyl, or (C₁-C₆)haloalkyl (e.g., CF₃), and m is 1. In certain other embodiments, each R₈ is independently H.

In another aspect, the present invention provides a pharmaceutical composition comprising at least one compound as described herein and a pharmaceutically-acceptable carrier or diluent.

In yet another aspect, the present invention provides a method for treating a psychotic disorder in a mammalian species in need thereof, the method comprising administering to the mammalian species a therapeutically effective amount of at least one compound as described herein, wherein the psychotic disorder is selected from schizophrenia, delusional disorders and drug induced psychosis.

In certain embodiments, the present invention provides a method for treating an anxiety disorder in a mammalian species in need thereof, the method comprising administering to the mammalian species a therapeutically effective amount of at least one compound as described herein, wherein the anxiety disorder is selected from panic disorder, agoraphobia, a specific phobia, social phobia, obsessive-compulsive disorder, post-traumatic stress disorder, acute stress disorder, and generalized anxiety disorder.

In certain embodiments, the present invention provides a method for treating a neurodegenerative disorder in a mammalian species in need thereof, the method comprising administering to the mammalian species a therapeutically effective amount of at least one compound as described herein, wherein the neurodegenerative disorder is selected from Parkinson's disease, Huntington's disease, dementia, Alzheimer's disease, multi-infarct dementia, AIDS-related dementia, Fronto temperal Dementia, neurodegeneration associated with cerebral trauma, neurodegeneration associated with stroke, neurodegeneration associated with cerebral infarct, hypoglycemia-induced neurodegeneration, neurodegeneration associated with epileptic seizure, neurodegeneration associated with neurotoxin poisoning, and multi-system atrophy.

### Utility and Methods of Use

Provided herein are methods for treating a disorder or disease by inhibiting PDE10 enzyme. The methods, in general, comprises the step of administering a therapeutically effective amount of at least one compound of the present invention, or a pharmaceutically acceptable salt thereof, to a patient in need thereof to treat the disorder or disease.

In certain embodiments, this invention provides a use of at least one compound as described herein in the manufacture of a medicament for treating a disorder or disease treatable by inhibition of PDE10.

The compounds of the present invention inhibit PDE 10 enzyme activity, in particular PDE10A enzyme activity and hence raise the levels of cAMP or cGMP within cells that express PDE10. Accordingly, inhibition of PDE10 enzyme activity may be useful in the treatment of diseases caused by deficient amounts of cAMP or cGMP in cells. PDE10 inhibitors may also be of benefit in cases wherein raising the amount of cAMP or cGMP above normal levels results in a therapeutic effect Inhibitors of PDE 10 may be used to treat disorders of the peripheral and central nervous system, cardiovascular diseases, cancer, gastro-enterological diseases, endocrinological diseases, metabolic diseases or urological diseases.

In certain embodiments, indications that may be treated with PDE 10 inhibitors, either alone or in combination with other drugs, include, but are not limited to, those diseases thought to be mediated in part by the basal ganglia, prefrontal cortex, and hippocampus.

In certain embodiments, these indications include psychoses, Parkinson's disease, dementias, obsessive compulsive disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease.

Psychoses are disorders that affect an individual's perception of reality. Psychoses are characterized by delusions and hallucinations. The compounds of the present invention are useful for treating patients suffering from all forms of psychoses, including, but are not limited to, schizophrenia, schizoaffective disorders, prodromal schizophrenia, late-onset schizophrenia, and bipolar disorders. Treatment can be for the positive symptoms of schizophrenia as well as for the cognitive deficits and negative symptoms. Other indications for PDE10 inhibitors include psychoses resulting from drug abuse (including amphetamines and PCP), encephalitis, alcoholism, epilepsy, Lupus, brain tumors, multiple sclerosis, dementia with Lewy bodies, sarcoidosis, or hypoglycemia. Other psychiatric disorders, like posttraumatic stress disorder (PTSD), and schizoid personality can also be treated with PDE10 inhibitors.

Obsessive-compulsive disorder (OCD) has been linked to deficits in the frontal-striatal neuronal pathways (Saxena et al., Br. J. Psychiatry Suppl, 35:26-37, 1998). Neurons in these pathways project to striatal neurons that express PDE10. PDE10 inhibitors cause cAMP to be elevated in these neurons; elevations in cAMP result in an increase in CREB phosphorylation and thereby improve the functional state of these neurons. The compounds of the present invention are therefore useful for the indication of OCD. OCD may result, in some cases, from streptococcal infections that cause autoimmune reactions in the basal ganglia (Giedd et al., Am J Psychiatry. 157:281-283, 2000). Because PDE10 inhibitors may serve a neuroprotective role, administration of PDE10 inhibitors may prevent the damage to the basal ganglia after repeated streptococcal infections and thereby prevent the development of OCD.

In the brain, the level of cAMP or cGMP within neurons is believed to be related to the quality of memory, especially long term memory. Without wishing to be bound to any particular theory, it is hypothesized that, since PDE10 degrades cAMP or cGMP, the level of this enzyme affects memory in animals, for example, in humans. A compound that inhibits cAMP phosphodiesterase (PDE) can thereby increase intracellular levels of cAMP, which in turn activate a protein kinase that phosphorylates a transcription factor (cAMP response binding protein). The phosphorylated transcription factor then binds to a DNA promoter sequence to activate genes that are important in long term memory. The more active such genes are, the better is long-term memory. Accordingly, by inhibiting a phosphodiesterase, long term memory can be enhanced.

Dementias are diseases that include memory loss and additional intellectual impairment separate from memory. The compounds of the present invention are useful for treating patients suffering from memory impairment in all forms of dementia. Dementias are classified according to their cause and include, e.g., neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, bacterial meningitis, Creutzfeld-Jacob Disease, psychiatric (e.g., depression and schizophrenia), and hydrocephalus.

The condition of memory impairment is manifested by impairment of the ability to learn new information and/or the inability to recall previously learned information. The present invention includes methods for dealing with memory loss separate from dementia, including mild cognitive impairment (MCI) and age-related cognitive decline. The present invention includes methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline. The compounds of the present invention are useful for treatment of memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, aging, head trauma, stroke, spinal cord injury, CNS hypoxia, cerebral senility, Creutzfeld-Jakob disease, depression, diabetes associated cognitive impairment, memory deficits from early exposure of anesthetic agents, multiinfarct dementia and other neurological conditions including acute neuronal diseases, as well as HIV and cardiovascular diseases.

The compounds of the present invention are also useful for treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins include, but are limited to, dentatorubral-pallidoluysian atrophy, D PLA (atrophin-1); spinocerebellar ataxia type-1 (ataxin-1); spinocerebellar ataxia type-2 (ataxin-2); spinocerebellar ataxia type-3 (also called Machado-Joseph disease or MJD) (ataxin-3); spinocerebellar ataxia type-6 (alpha la-voltage dependent calcium channel); spinocerebellar ataxia type-7 (ataxin-7); and spinal and bulbar muscular atrophy (SBMA, also known as Kennedy disease).

The basal ganglia are important for regulating the function of motor neurons; disorders of the basal ganglia result in movement disorders. Most prominent among the movement disorders related to basal ganglia function is Parkinson's disease (Obeso et al, Neurology. 62(1 Suppl 1):S17-30, 2004). Other movement disorders related to dysfunction of the basal ganglia include tardive dyskinesia, progressive supranuclear palsy and cerebral palsy, corticobasal degeneration, multiple system atrophy, dystonia, tics, Wilson disease, and chorea. The compounds of the invention are also useful for treatment of movement disorders related to dysfunction of basal ganglia neurons.

PDE10 inhibitors are useful in raising cAMP or cGMP levels and prevent neurons from undergoing apoptosis. PDE10 inhibitors may be anti-inflammatory by raising cAMP in glial cells. The combination of anti-apoptotic and anti-inflammatory properties, as well as positive effects on synaptic plasticity and neurogenesis, make these compounds useful to treat neurodegeneration resulting from any disease or injury, including stroke, spinal cord injury, Alzheimer's disease, amylolaterosclerosis (ALS), multiple sclerosis, and multiple systems atrophy (MSA).

Autoimmune diseases or infectious diseases that affect the basal ganglia may result in disorders of the basal ganglia including ADHD, OCD, tics, Tourette's disease, and Sydenham chorea. In addition, any insult to the brain can potentially damage the basal ganglia including strokes, metabolic abnormalities, liver disease, multiple sclerosis, infections, tumors, drug overdoses or side effects, and head trauma. Therefore, the compounds of the invention can be used to hinder or delay disease progression or restore damaged circuits in the brain by a combination of effects including increased synaptic plasticity, anti-inflammatory, nerve cell regeneration, neurogenesis, and decreased apoptosis.

The growth of some cancer cells is inhibited by cAMP and cGMP. Upon transformation, cells may become cancerous by expressing PDE10 and reducing the amount of cAMP or cGMP within cells. In these types of cancer cells, inhibition of PDE10 activity inhibits cell growth by raising cAMP. In some cases, PDE10 may be expressed in the transformed, cancerous cell but not in the parent cell line. In transformed renal carcinoma cells, PDE10 is expressed and PDE10 inhibitors reduce the growth rate of the cells in culture. Similarly, breast cancer cells are inhibited by administration of PDE10 inhibitors. Many other types of cancer cells may also be sensitive to growth arrest by inhibition of PDE10. Accordingly, compounds disclosed in this invention can be used to inhibit the growth of cancer cells that express PDE10.

The compounds of the invention are also useful for treatment of diabetes and related disorders such as obesity, via regulating the cAMP signaling system. By inhibiting PDE-10, especially PDE10A, intracellular levels of cAMP are increased, thereby increasing the release of insulin-containing secretory granules and, therefore, increasing insulin secretion.

In certain embodiments, the indications that may be treated with PDE10 inhibitors include neurological and psychiatric disorders selected from psychotic disorders and conditions; anxiety disorders; movement disorders; drug abuse; mood disorders; neurodegenerative disorders; disorders or conditions comprising as a symptom a deficiency in attention and/or cognition; pain and metabolic disorders.

In certain embodiments, the psychotic disorders and conditions associated with PDE10 dysfunction include one or more of the following conditions or diseases: schizophrenia, for example of the paranoid, disorganized, catatonic, undifferentiated or residual type; schizophreniform disorder; schizoaffective disorder, such as delusional or depressive type; delusional disorder; substance-induced psychotic disorder such as psychosis induced by alcohol, amphetamine, cannabis, cocaine, hallucinogens, inhalants, opioids, or phencyclidine; personality disorders of the paranoid type; and personality disorder of the schizoid type.

In certain embodiments, the anxiety disorders include panic disorder; agoraphobia; specific phobia; social phobia; obsessive-compulsive disorder; post-traumatic stress disorder; acute stress disorder; and generalized anxiety disorder.

In certain embodiments, movement disorders include Huntington's disease and dyskinesia; Parkinson's disease; restless leg syndrome and essential tremor. In addition, Tourette's syndrome and other tic disorders can be included.

In certain embodiments, the central nervous system disorder is a substance-related disorder selected from the group of alcohol abuse; alcohol dependence; alcohol withdrawal; alcohol withdrawal delirium; alcohol-induced psychotic disorder; amphetamine dependence; amphetamine withdrawal; cocaine dependence; cocaine withdrawal; nicotine dependence; nicotine withdrawal; opioid dependence and opioid withdrawal.

In certain embodiments, mood disorders and mood episodes include depression, mania and bipolar disorders. Preferably, the mood disorder is selected from the group of bipolar disorders (I and II); cyclothymic disorder; depression; dysthymic disorder; major depressive disorder and substance-induced mood disorder.

In certain embodiments, neurodegenerative disorders include Parkinson's disease; Huntington's disease; dementia such as for example Alzheimer's disease; multi-infarct dementia; AIDS-related dementia or fronto temperal dementia. The neurodegenerative disorder or condition comprises neurodegeneration of striatal medium spiny neurons.

In certain embodiments, disorders or conditions comprising as a symptom a deficiency in attention and/or cognition include dementia, such as Alzheimer's disease; multi-infarct dementia; alcoholic dementia or drug-related dementia; dementia associated with intracranial tumours or cerebral trauma; dementia associated with Huntington's disease; dementia associated with Parkinson's disease; AIDS-related dementia; other diseases include delirium; amnestic disorder; post-traumatic stress disorder; mental retardation; a learning disorder; attention-deficit/hyperactivity disorder (ADHD); and age-related cognitive impairment.

In certain embodiments, pain includes acute and chronic states, severe pain, intractable pain, neuropathic pain and post-traumatic pain.

In certain embodiments, metabolic disorders include diabetes, in particular type 1 or type 2 diabetes, and related disorders such as obesity. Additional related disorders include syndrome X, impaired glucose tolerance, impaired fasting glucose, gestational diabetes, maturity-onset diabetes of the young (MODY), latent autoimmune diabetes adult (LADA), associated diabetic dyslipidemia, hyperglycemia, hyperinsulinemia, dyslipidemia, hypertriglyceridemia, and insulin resistance.

Further, the growth of some cancer cells is inhibited by cAMP and cGMP, the compounds of the invention may be useful in the treatment of cancer, such as renal carcinoma and breast cancer.

In certain embodiments, the psychotic disorder is selected from the group of schizophrenia, delusional disorder, schizoaffective disorder, schizophreniform disorder and substance-induced psychotic disorder.

In certain embodiments, the central nervous system disorder is a personality disorder selected from the group of obsessive-compulsive personality disorder and schizoid, schizotypal disorder.

In certain embodiments, the central nervous system disorder is a mood disorder selected from the group of bipolar disorders (I & II), cyclothymic disorder, depression, dysthymic disorder, major depressive disorder and substance-induced mood disorder.

In certain embodiments, the central nervous system disorder is attention-deficit/hyperactivity disorder.

In certain embodiments, the central nervous system disorder is a cognitive disorder selected from the group of delirium, substance-induced persisting delirium, dementia, dementia due to HIV disease, dementia due to Huntington's disease, dementia due to Parkinson's disease, dementia of the Alzheimer's type, substance-induced persisting dementia and mild cognitive impairment.

In certain embodiments, the disorders treated by the compounds of the present invention are selected from schizophrenia; obsessive-compulsive disorder; generalized anxiety disorder; Huntington's disease; dyskinesia; Parkinson's disease; depression; bipolar disorders; dementia such as Alzheimer's disease; attention-deficit/hyperactivity disorder; drug abuse; pain; diabetes and obesity.

In certain embodiments, the disorders treated by the compounds of the present invention are schizophrenia, including positive and negative symptoms thereof, and cognitive deficits, such as impaired attention or memory.

In certain embodiments, the compounds of the present invention are particularly useful for treatment of anxiety, obsessive-compulsive disorder, schizophrenia, depression, attention-deficit/hyperactivity disorder, Alzheimer's disease and diabetes.

In certain embodiments, the invention also relates to the use of a compound according to the invention, for the manufacture of a medicament for the treatment or prevention of any one of the disease conditions mentioned hereinbefore.

In view of the utility of the compounds according to the invention, there is provided a method of treating warm-blooded animals, including humans, suffering from any one of the diseases mentioned hereinbefore, and a method of preventing in warm-blooded animals, including humans, any one of the diseases mentioned hereinbefore.

Said methods comprise the administration, i.e. the systemic or topical administration, preferably oral administration, of a therapeutically effective amount of a compound according to the invention to warm-blooded animals, including humans.

Therefore, the invention also relates to a method for the prevention and/or treatment of any one of the diseases mentioned hereinbefore comprising administering a therapeutically effective amount of compound according to the invention to a patient in need thereof.

The PDE10 inhibitors described herein can be used alone, in combination or in combination with other pharmaceutical agents such as other agents used in the treatment of psychoses, such as schizophrenia and bipolar disorder, obsessive-compulsive disorder, Parkinson's disease, cognitive impairment and/or memory loss, e.g. nicotinic α-7 agonists, PDE4 inhibitors, other PDE10 inhibitors, calcium channel blockers, muscarinic m1 and m2 modulators, adenosine receptor modulators, ampakines, NMDA-R modulators, mGluR modulators, dopamine modulators, serotonin modulators, cannabinoid modulators, and cholinesterase inhibitors (e.g. donepezil, rivastigmine, and galantamine). In such combinations, the compounds of the present invention may be utilized in combination with one or more other drugs in the treatment, prevention, control, amelioration, or reduction of risk of diseases or conditions for which compounds of Formula (I) or the other drugs may have utility, where the combination of the drugs together are safer or more effective than either drug alone.

One skilled in the art will recognize that a therapeutically effective amount of the PDE10 inhibitors of the present invention is the amount sufficient to inhibit the PDE10 enzyme and that this amount varies inter alia, depending on the type of disease, the concentration of the compound in the therapeutic formulation, and the condition of the patient. Generally, an amount of PDE10 inhibitor to be administered as a therapeutic agent for treating diseases in which inhibition of the PDE10 enzyme is beneficial, such as the disorders described herein, will be determined on a case by case by an attending physician.

Generally, a suitable dose is one that results in a concentration of the PDE10 inhibitor at the treatment site in the range of 0.5 nM to 200 μM, and more usually 5 nM to 50 μM. To obtain these treatment concentrations, a patient in need of treatment likely will be administered between 0.001 mg/kg to 15 mg/kg body weight, in particular from 0.01 mg/kg to 2.50 mg/kg body weight, in particular, from 0.01 to 1.5 mg/kg body weight, in particular from 0.1 mg/kg to 0.50 mg/kg body weight. The amount of a compound according to the present invention, also referred to here as the active ingredient, which is required to achieve a therapeutic effect may vary on case-by-case basis, vary with the particular compound, the route of administration, the age and condition of the recipient, and the particular disorder or disease being treated. A method of treatment may also include administering the active ingredient on a regimen of between one and four intakes per day. In these methods of treatment the compounds according to the invention are preferably formulated prior to admission. As described herein below, suitable pharmaceutical formulations are prepared by known procedures using well known and readily available ingredients.

### Methods of Preparation:

### ABBREVIATIONS

### ACN Acetonitrile

EA Ethyl acetate
DMF Dimethyl formamide
PE Petroleum ether

### DCM Dichloromethane

### THF Tetrahydrofuran

EDCI 1-Ethyl-3-(3-dimethylaminopropyl)carbodiimide
DMF-DMA N,N-Dimethylformamide dimethyl acetal

### TEA Triethylamine

### DIPEA Diisopropylethylamine

### DMAP 4-Dimethylaminopyridine

### NBS N-bromosuccinimide

### NCS N-chlorosuccinimide

### NIS N-iodosuccinimide

TsOH p-Toluenesulfonic acid
TFA Trifluoroacetic acid

Following are general synthetic schemes for manufacturing compounds of the present invention. These schemes are illustrative and are not meant to limit the possible techniques one skilled in the art any use to manufacture compounds disclosed herein. Different methods will be evident to those skilled in the art. Additionally, the various steps in the synthesis may be performed in an alternate sequence or order to give the desired compound(s). All documents cited herein are incorporated herein by reference in their entirety. For example, the following reactions are illustrations but not limitations of the preparation of some of the starting materials and examples used herein.

Schemes 1-16 describe various methods for the synthesis of the compounds of the present invention. Various modifications to these methods may be envisioned by those skilled in the art to achieve similar results to that of the inventors given below. In these schemes, the symbols are as defined in the claims and specification unless otherwise noted.

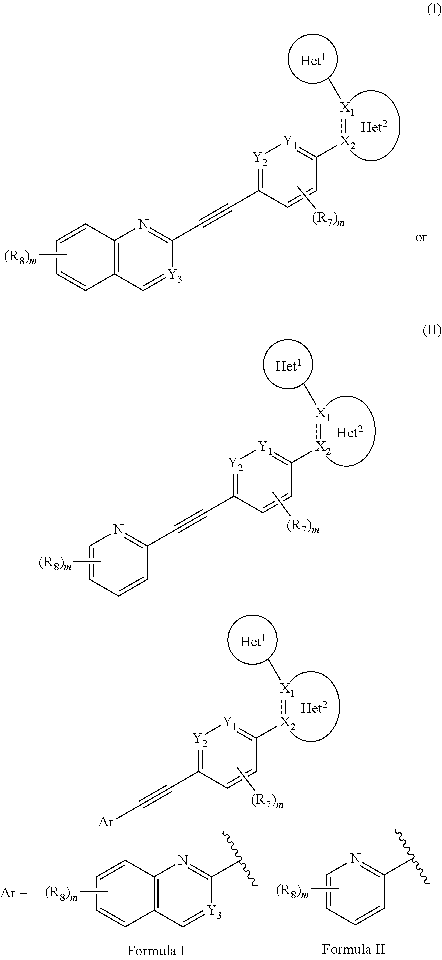

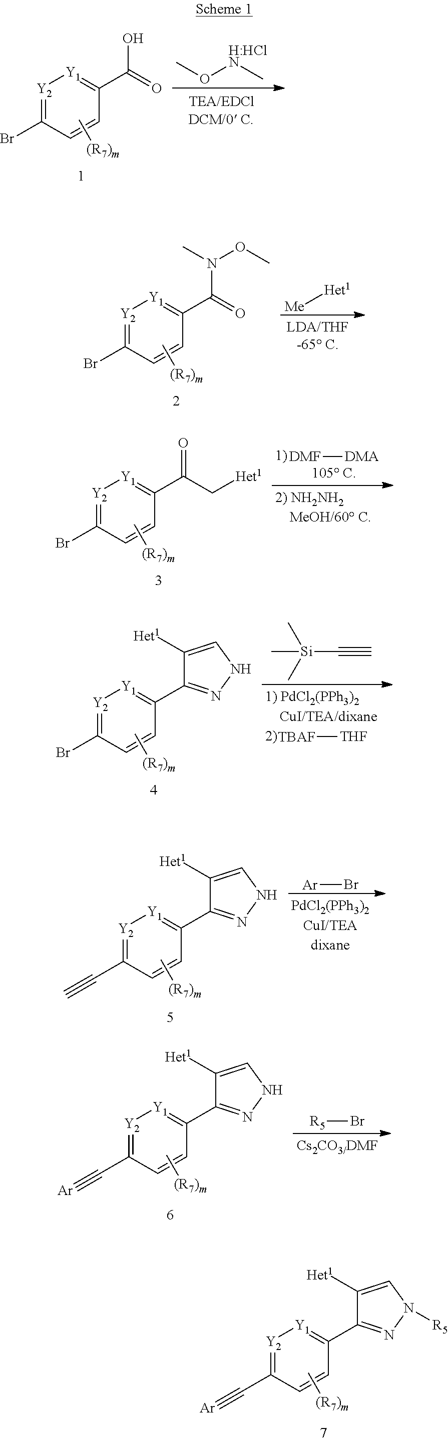

Compounds of Formulae (I) and (II) in which Het¹=aryl or heteroaryl, and compounds of Formulae (III) and (IV) may be prepared according to Scheme 1.

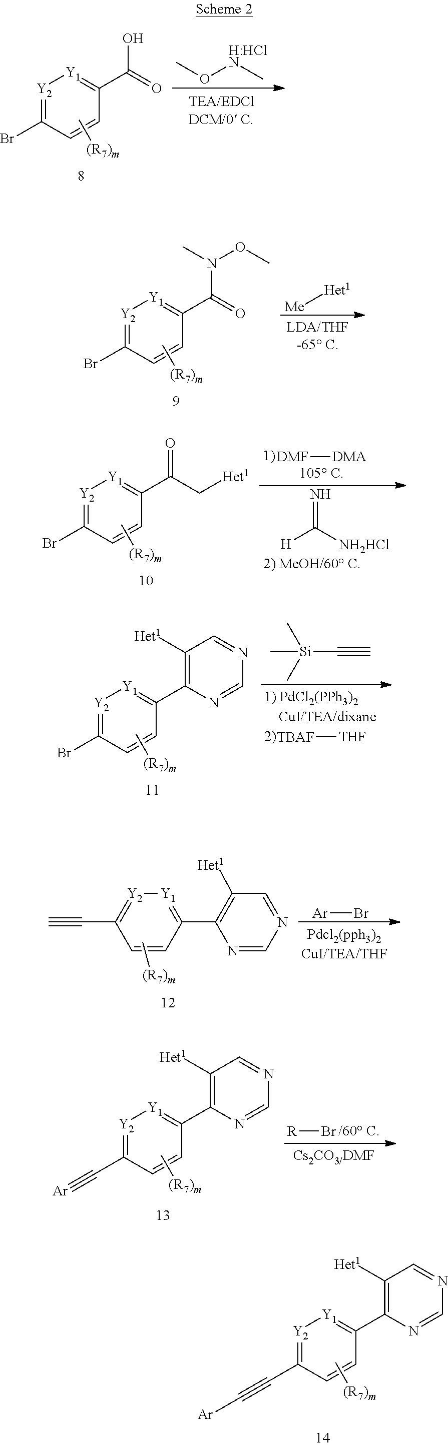

Compounds of Formulae (I) and (II) in which Het¹=aryl or heteroaryl, and compounds of Formulae (VII) and (VIII) may be prepared according to Scheme 2.

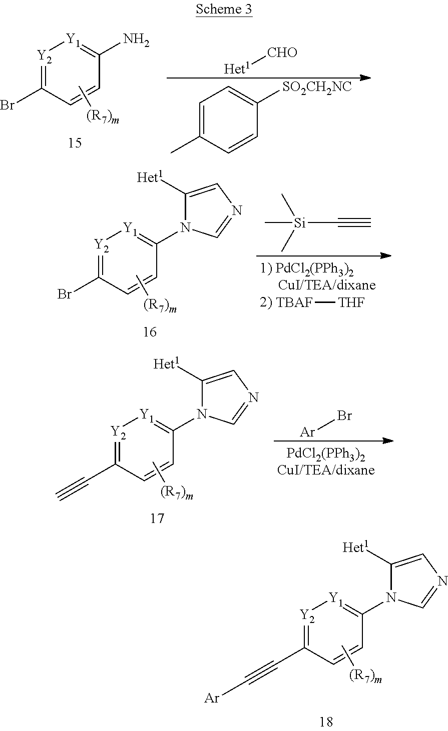

Compounds of Formulae (I) and (II) in which Het¹=aryl or heteroaryl, and compound of Formulae (XIII) and (XIV) may be prepared according to Scheme 3.

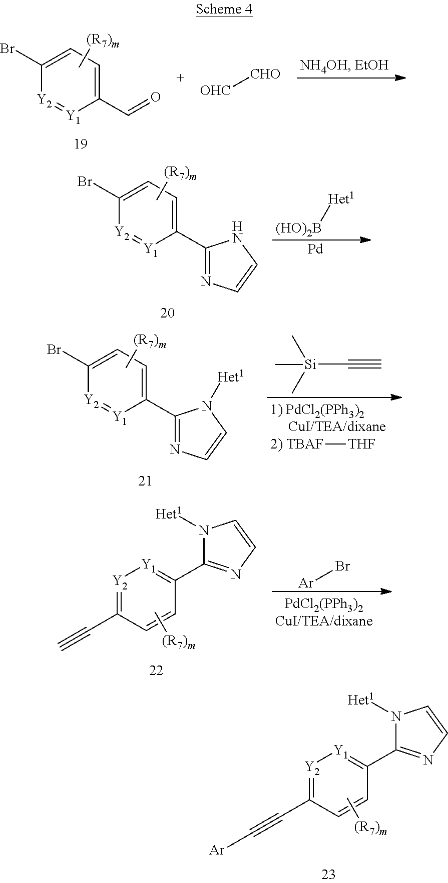

Compounds of Formulae (I) and (II) in which Het¹=aryl or heteroaryl, may be prepared according to Scheme 4.

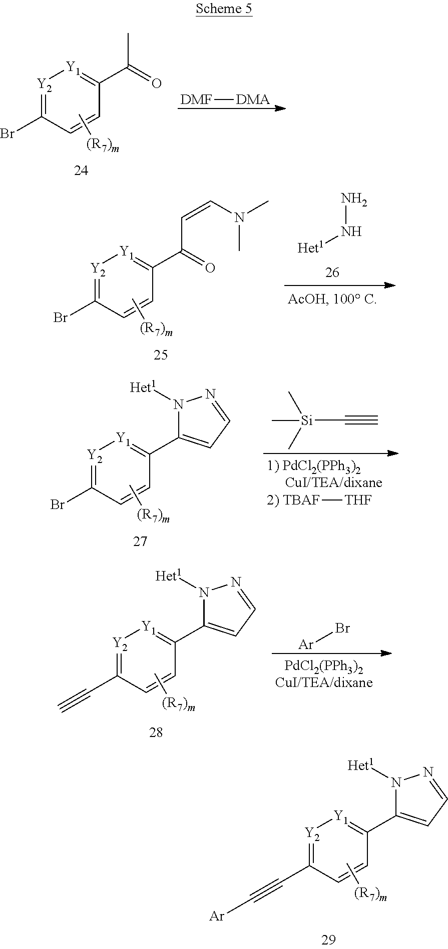

Compounds of Formulae (I) and (II) in which Het¹=aryl or heteroaryl, and compounds of Formulae (XI) and (XII) may be prepared according to Scheme 5.

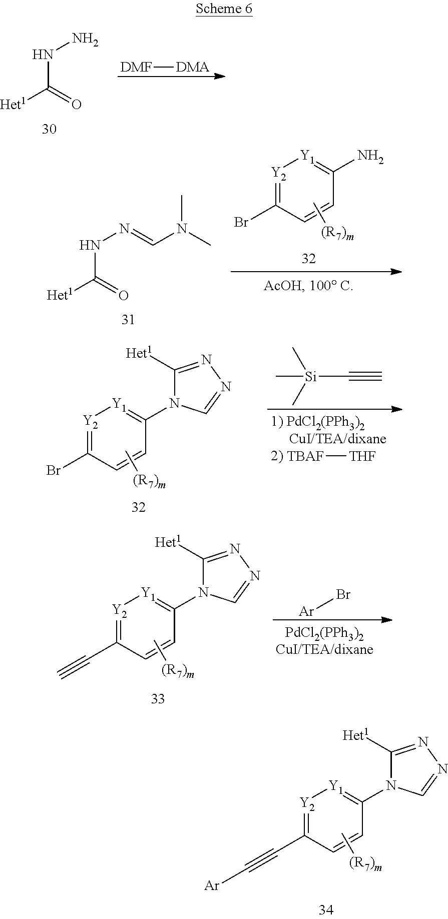

Compounds of Formulae (I) and (II) in which Het¹=aryl or heteroaryl, and compounds of Formulae (XV) and (XVI) may be prepared according to Scheme 6.

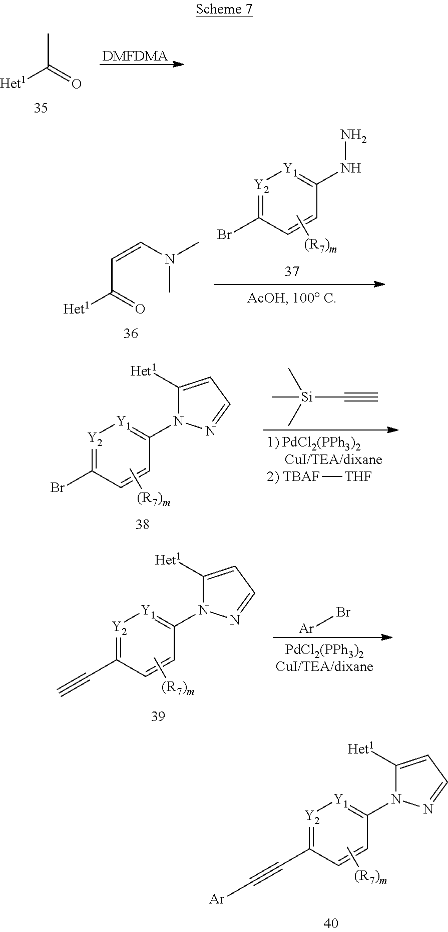

Compounds of Formulae (I) and (II) in which Het¹=aryl or heteroaryl, and compounds of Formulae (IXX) and (XX) may be prepared according to Scheme 7.

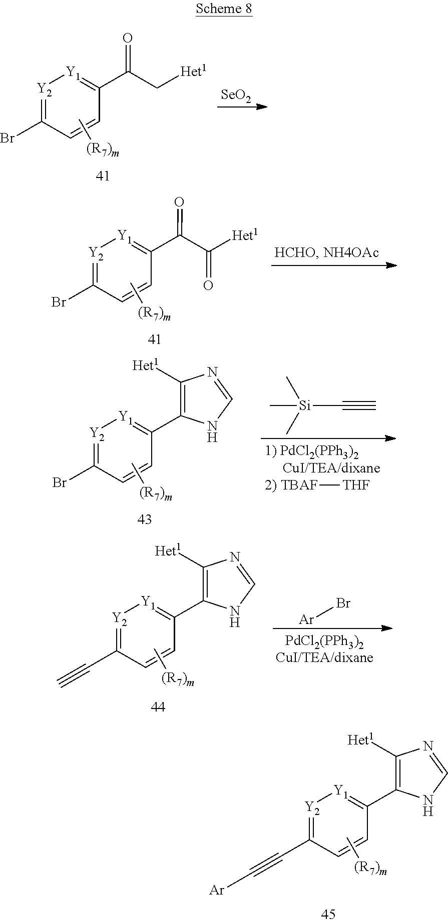

Compounds of Formulae (I) and (II) in which Het¹=aryl or heteroaryl, may be prepared according to Scheme 8.

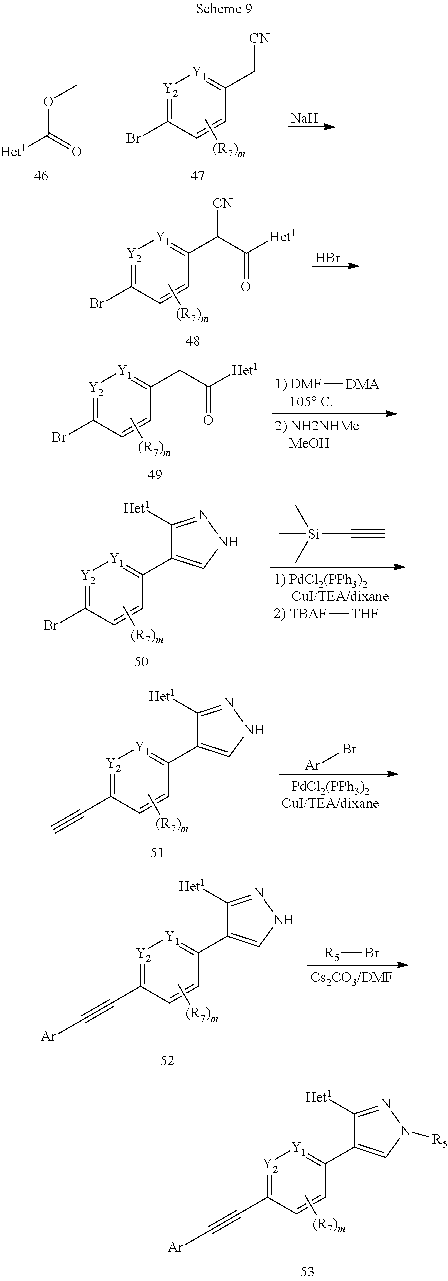

Compounds of Formulae (I) and (II) in which Het¹=aryl or heteroaryl, and compounds of Formulae (XVII) and (XVIII) may be prepared according to Scheme 9.

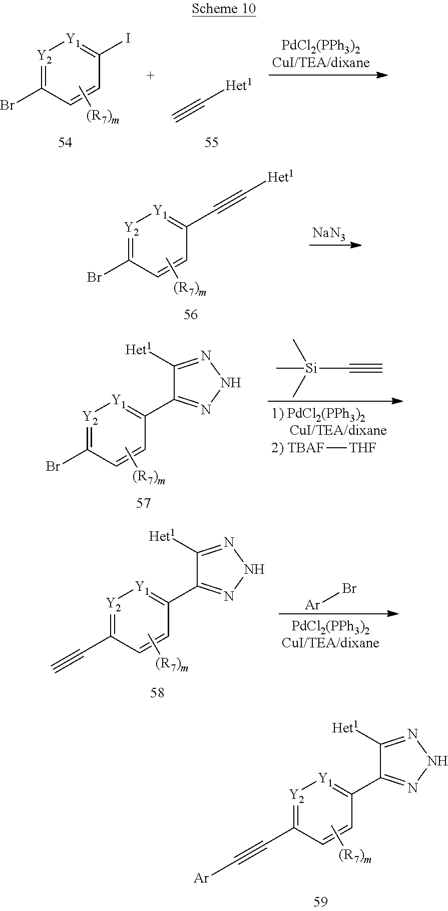

Compounds of Formulae (I) and (II) in which Het¹=aryl or heteroaryl, may be prepared according to Scheme 10.

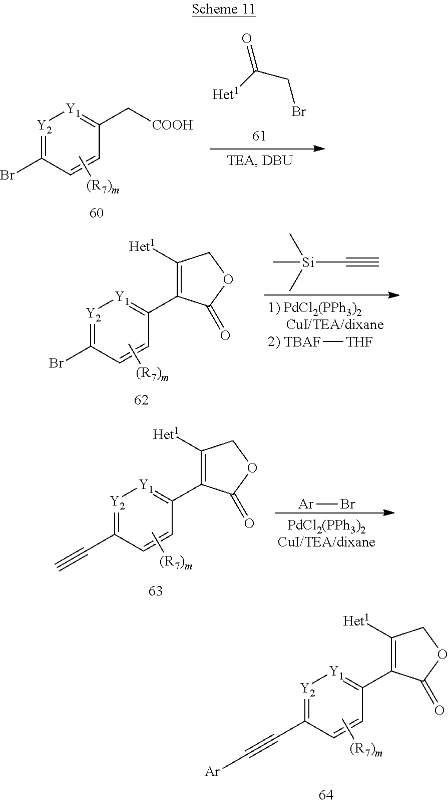

Compounds of Formulae (I) and (II) in which Het¹=aryl or heteroaryl, may be prepared according to Scheme 11.

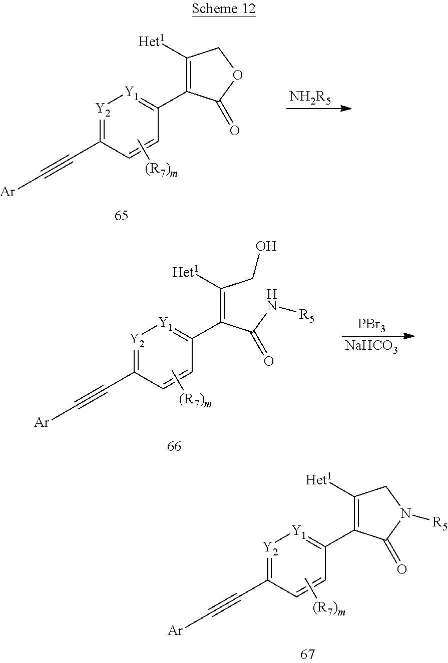

Compounds of Formulae (I) and (II) in which Het¹=aryl or heteroaryl, may be prepared according to Scheme 12.

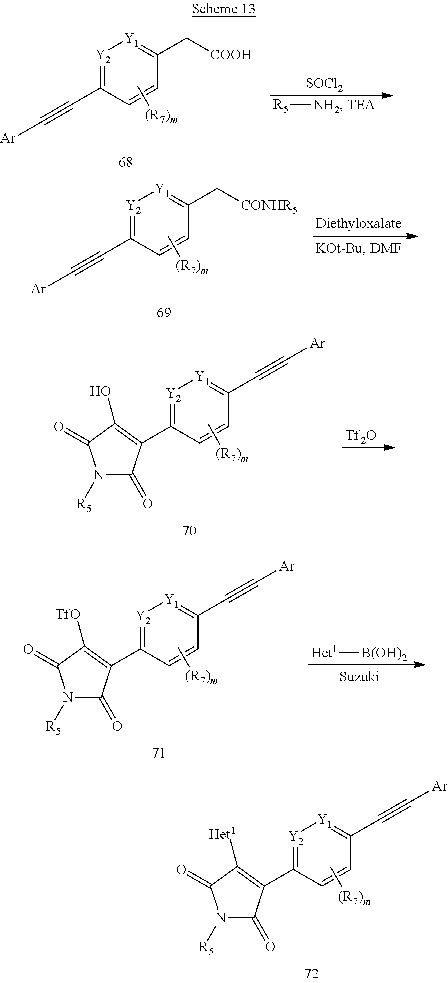

Compounds of Formulae (I) and (II) in which Het¹=aryl or heteroaryl, may be prepared according to Scheme 13.

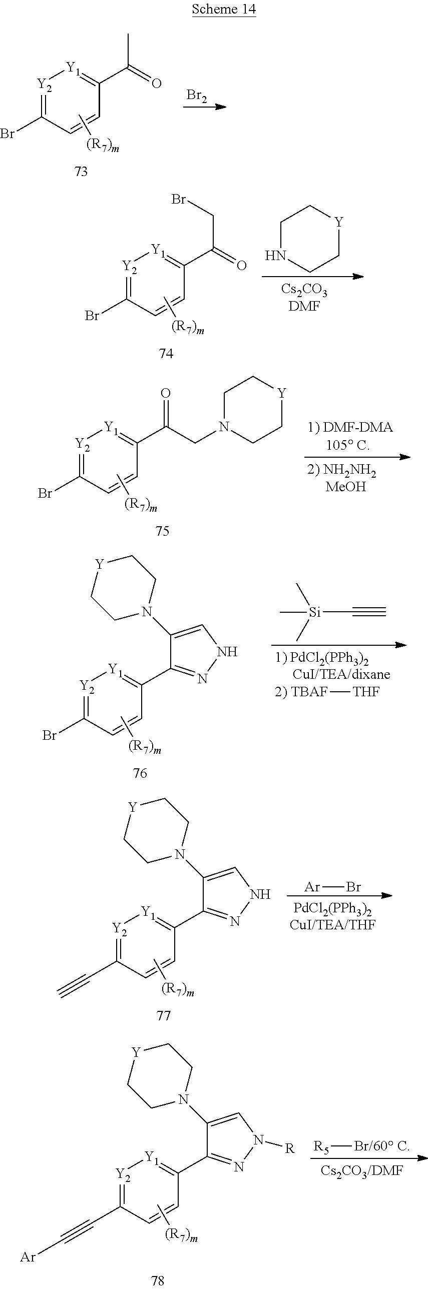

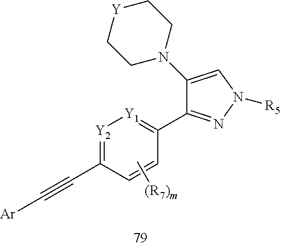

Compounds of Formulae (I) and (II) in which Het¹=aryl or heteroaryl, may be prepared according to Scheme 14.

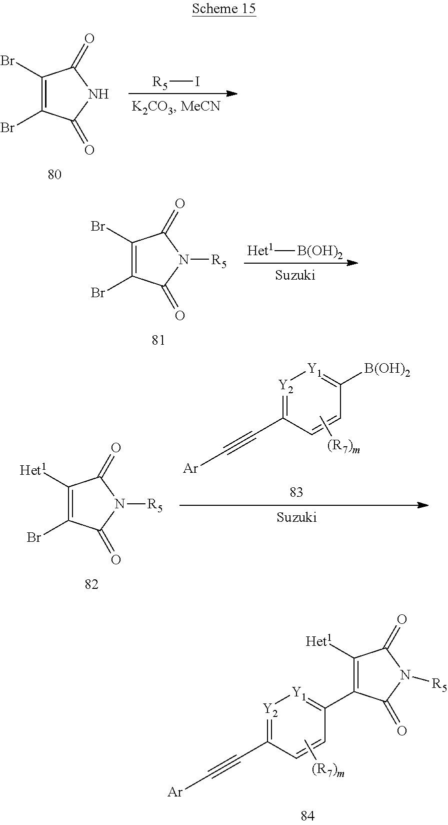

Compounds of Formulae (I) and (II) in which Het¹=aryl or heteroaryl, may be prepared according to Scheme 15.

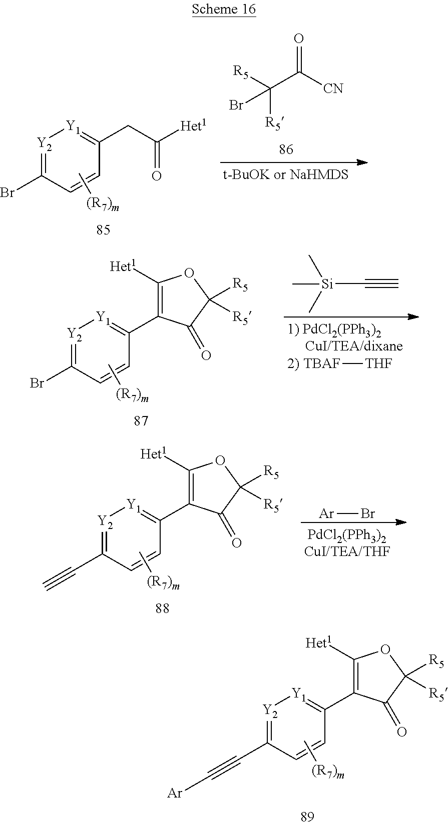

Compounds of Formulae (I) and (II) in which Het¹=aryl or heteroaryl, may be prepared according to Scheme 16.

### Pharmaceutical Compositions

This invention also provides a pharmaceutical composition comprising at least one of the compounds as described herein or a pharmaceutically-acceptable salt thereof, and a pharmaceutically-acceptable carrier.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As set out above, certain embodiments of the present pharmaceutical agents may be provided in the form of pharmaceutically-acceptable salts. The term “pharmaceutically-acceptable salt”, in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al., (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate, magnesium stearate, and polyethylene oxide-polybutylene oxide copolymer as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets, may be, made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying butortions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples are embedding compositions, which can be used include polymeric substances and waxes. The active ingredient can also be in micro-encapsulated form, if apbutriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-.beta.-cyclodextrin, may be used to solubilize compounds.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active pharmaceutical agents of the invention.

Formulations of the present invention which are suitable for vaginal administration also include pessaries, tampons, creams, gels, pastes, foams or spray formulations containing such carriers as are known in the art to be apbutriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or butellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary butellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving, or dispersing the pharmaceutical agents in the buter medium. Absorption enhancers can also be used to increase the flux of the pharmaceutical agents of the invention across the skin. The rate of such flux can be controlled, by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polybutylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anti-HCV agent), or they may achieve different effects (e.g., control of any adverse effects).

The compounds of the invention may be administered intravenously, intramuscularly, intraperitoneally, subcutaneously, topically, orally, or by other acceptable means. The compounds may be used to treat arthritic conditions in mammals (i.e., humans, livestock, and domestic animals), birds, lizards, and any other organism, which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and equivalents thereof.

### EXAMPLES

### HPLC Condition

### Method A:

### Column: Chromolith SpeedROD, RP-18e, 50*4.6 mm

### Mobile Phase: A: CH3CN/H2O/HCOOH=10/90/0.05

### B: CH3CN/H2O/HCOOH=90/10/0.05

### Temperature: 40° C.

Flow rate: 3 mL/min
Run time: 0.8 min@10% B, 2.7 min gradient (10-95% B), then 0.8 min@95% B
Injection volume: 1 uL
Mass range: 100-1000

### Detector: UV, Wavelength: 254/220 nm

### Method B:

### Column: Chromolith SpeedROD, RP-18e, 50×4.6 mm

### Mobile Phase Solvent A: CH3CN/H2O/FA=10/90/0.05

### Solvent B: CH3CN/H2O/FA=90/10/0.05

### Temperature: 40° C.

Flow rate: 3 mL/min.
Run time: 0.8 min@ 10% B, 2.7 min gradient (10-95% B), then 0.8 min@95% B.

### Detector: UV, Wavelength: 254/220 nm

### Example 1

### 2-((4-(1-methyl-4-(pyridin-4-yl)-1H-pyrazol-3-yl)phenyl)ethynyl)quinoline

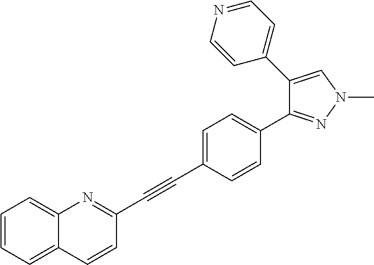 [SMILES: Cn1cc(-c2ccncc2)c(-c2ccc(C#Cc3ccc4ccccc4n3)cc2)n1]

### Example 1A

### Preparation of 1-(4-bromophenyl)-2-(pyridin-4-yl)ethanone

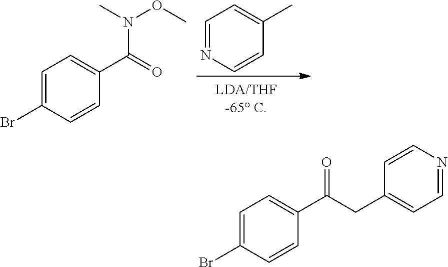

To a solution of LDA (51 ml, 2M in THF) in THF (50 mL), was slowly added 4-methylpyridine (9.5 g, 102 mmol) at 0° C. under N₂. After 30 minutes the resulting anion was cooled to −65° C. In a separate round bottom flask, 4-bromo-N-methoxy-N-methylbenzamide was dissolved in THF (50 ml) and cooled to −65° C. under N₂. To this, a freshly prepared anion (1.2 eq) solution was slowly added over a period of 45 minutes. After an additional 30 minutes, AcOH (50 ml) was slowly added and the reaction was allowed to warm up to room temperature. The mixture was basified to pH7 and extracted with EA. The organic extract was washed with brine, dried over Na₂SO₄, filtered and concentrated. The residue was recrystallized from (PE:EA=10:1) to give the titled product as a yellow solid (5.2 g, yield: 92%); MS (ESI) m/z=276 [M+H]⁺.

### Example 1B

### Preparation of 4-(3-(4-bromophenyl)-1-methyl-1H-pyrazol-4-yl)pyridine

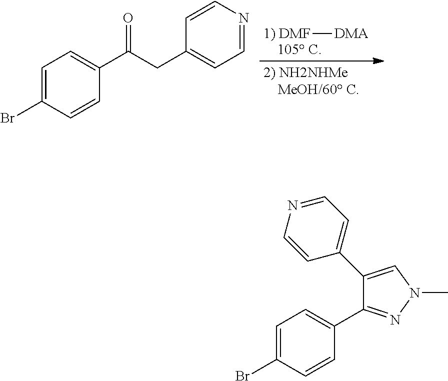

1-(4-bromophenyl)-2-(pyridin-4-yl)ethanone (4 g, 14.5 mmol) was heated at reflux in DMF-DMA (13 ml) for 1 hour and concentrated. The residue was dissolved in MeOH, and to this, methylhydrazine in water (40%) (3 ml) was added. Then the reaction mixture was heated at 65° C. for 1 hour. When LC/MS indicated the reaction was completed. The mixture was concentrated and purified by column chromatography over silica gel using (PE:EA=10:1-1:1) to give 3 g of crude product. The crude product was recrystallized from (PE:EA=50:1) to give product as a white solid (2.3 g, yield: 50%); MS (ESI) m/z=314 [M+H]⁺.

### Example 1C

### Preparation of

### 4-(1-methyl-3-(4-((trimethylsilyl)ethynyl)phenyl)-1H-pyrazol-4-yl)pyridine

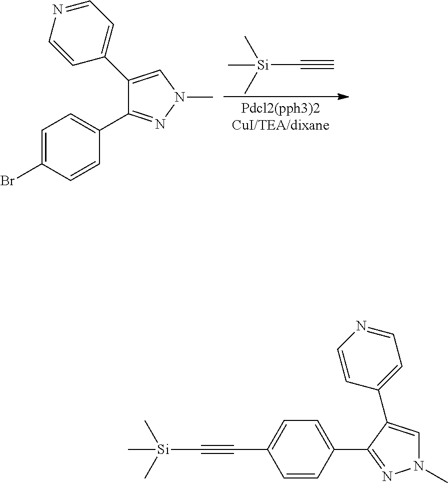

A solution of 4-(3-(4-bromophenyl)-1-methyl-1H-pyrazol-4-yl)pyridine (2.0 g, 6.4 mmol), CuI (60.8 mg, 0.32 mmol), PdCl₂(PPh₃)₂ (225 mg, 0.32 mmol) in TEA/dioxane (2:1) was degassed with N₂ at room temperature for 5 minutes. To this, was added ethynyltrimethylsilane (1.87 g, 19.1 mmol), and the resulting reaction mixture was again degassed for 2 minutes. Then the reaction mixture was heated at 100° C. for 1 hour under N₂. When LC/MS indicated the reaction was completed. The mixture was concentrated and purified by column chromatography over silica gel using (PE:EA=1:1) to give product as a white solid. (2.0 g, yield: 90%); MS (ESI) m/z=332 [M+H]⁺.

### Example 1D

### Preparation of 4-(3-(4-ethynylphenyl)-1-methyl-1H-pyrazol-4-yl)pyridine

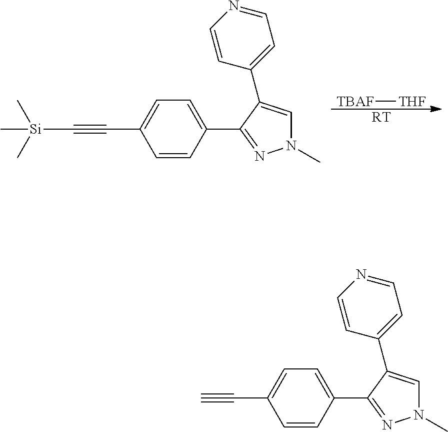

To a solution of 4-(1-methyl-3-(4-((trimethylsilyl)ethynyl)phenyl)-1H-pyrazol-4-yl)-pyridine in THF (10 ml) was added TBAF/THF (10 ml, 1M in THF) at 0° C. The reaction mixture was stirred at room temperature for 1 hour. When LC/MS indicated that the reaction was completed. The solution was then diluted with EA and washed with water, and brine. The organic extracts were dried over Na₂CO₃ and concentrated. The residue was purified by column chromatography over silica gel using (PE:EA=1:2) to give the product as a white solid (1.0 g, yield: 64%); MS (ESI) m/z=260 [M+H]⁺.

### Preparation of 2-((4-(1-methyl-4-(pyridin-4-yl)-1H-pyrazol-3-yl)phenyl)ethynyl)quinoline

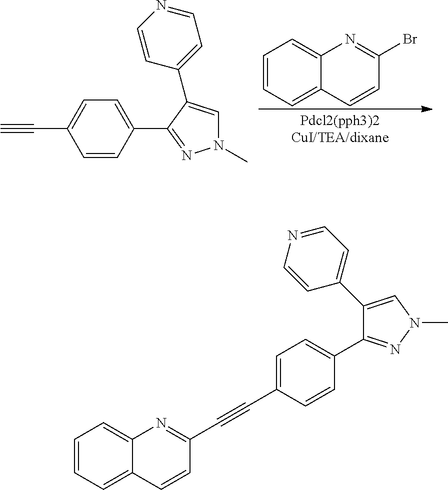 [SMILES: C#Cc1ccc(-c2nn(C)cc2-c2ccncc2)cc1 ; Brc1ccc2ccccc2n1 ; Cn1cc(-c2ccncc2)c(-c2ccc(C#Cc3ccc4ccccc4n3)cc2)n1]

A solution of 2-bromoquinoline (48 mg, 0.23 mmol), CuI (2.2 mg, 0.0116 mmol), PdCl₂(PPh₃)₂ (8 mg, 0.0116 mmol) in TEA/dioxane (2 ml, 2:1) was degassed with N₂ at room temperature for 5 minutes. To this, was added 4-(3-(4-ethynylphenyl)-1-methyl-1H-pyrazol-4-yl)pyridine (60 mg, 0.23 mmol) and the resulting reaction mixture was again degassed for 2 minutes. Then the reaction mixture was heated at 100° C. for 1 hour under N₂.

When LC/MS indicated the reaction was completed. The mixture was concentrated and purified by column chromatography over silica gel using (PE:EA=1:1) to give the desired product as a yellow solid (40 mg, Y: 45%); ¹H NMR (400 MHz, CD₃OD) δ 8.53 (s, 2H), 8.12-8.16 (m, 2H), 7.50-7.82 (m, 9H), 7.19 (s, 2H), 4.01 (s, 3H); MS (ESI) m/z=387 [M+H]⁺; HPLC retention time: 1.91 min (Method A).

### Example 2

### 2-((4-(1-methyl-4-(pyridin-4-yl)-1H-pyrazol-3-yl)phenyl)ethynyl)quinazoline

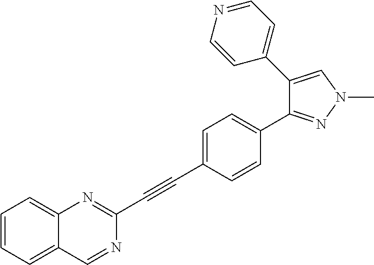 [SMILES: Cn1cc(-c2ccncc2)c(-c2ccc(C#Cc3ncc4ccccc4n3)cc2)n1]

Example 2 was prepared using a method analogous to that of Example 1 utilizing Example 1D and 2-chloroquinazoline, as a white solid in 34% yield; ¹H NMR (400 MHz, CD₃OD) δ 9.58 (s, 1H), 8.03-8.20 (m, 5H), 7.75-7.85 (m, 4H), 7.56-7.58 (m, 2H), 7.44 (brs, 2H), 4.03 (s, 3H), MS (ESI) m/z=388 [M+H]⁺; HPLC retention time: 1.71 minutes (Method A).

### Example 3

### 2-methyl-6-(4-(1-methyl-4-(pyridin-4-yl)-1H-pyrazol-3-yl)phenyl)ethynyl)pyridine

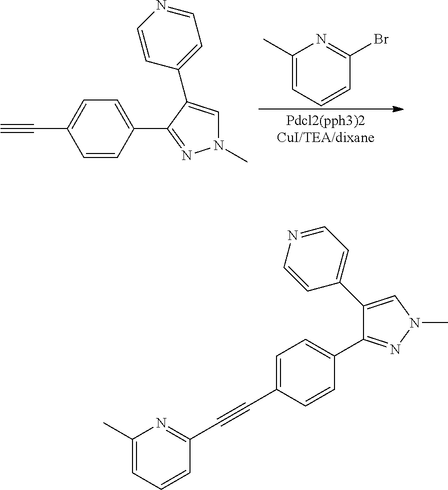

Example 3 was prepared using a method analogous to that of Example 1 utilizing Example 1D and 2-bromo-6-methylpyridine, as a white solid in 45% yield; ¹H NMR (400 MHz, CDCl₃) δ 8.56 (brs, 2H), 8.48 (s, 1H), 7.59-7.62 (m, 3H), 7.47-7.51 (m, 4H), 7.22 (br, s, 2H), 7.22 (br, s, 2H), 4.03 (s, 3H); 2.39 (s, 3H); MS (ESI) m/z=351 [M+H]⁺; HPLC retention time: 1.47 minutes (Method A).

### Example 4

### 2-Ethyl-6-((4-(1-methyl-4-(pyridin-4-yl)-1H-pyrazol-3-yl)phenyl)ethynyl)pyridine

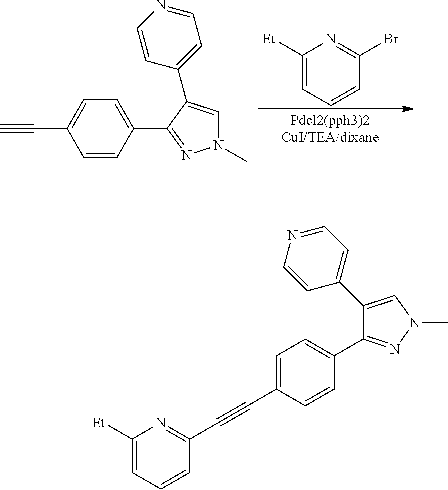 [SMILES: C#Cc1ccc(-c2nn(C)cc2-c2ccncc2)cc1 ; CCc1cccc(Br)n1 ; CCc1cccc(C#Cc2ccc(-c3nn(C)cc3-c3ccncc3)cc2)n1]

Example 4 was prepared using a method analogous to that of Example 1 utilizing Example 1D and 2-bromo-6-ethylpyridine, as a white solid in 24% yield; ¹H NMR (400 MHz, CDCl₃) δ 7.48-7.64 (m, 10H), 7.38-7.40 (d, J=8 Hz, 1H), 7.15-7.17 (d, J=8 Hz, 1H), 4.04 (s, 3H), 2.85-2.91 (q, 2H), 1.27-1.36 (t, 3H); MS (ESI) m/z=365 [M+H]⁺; HPLC retention time: 1.68 minutes (Method A).

### Example 5

### 2-cyclopropyl-6-((4-(1-methyl-4-(pyridin-4-yl)-1H-pyrazol-3-yl)phenyl)ethynyl)pyridine

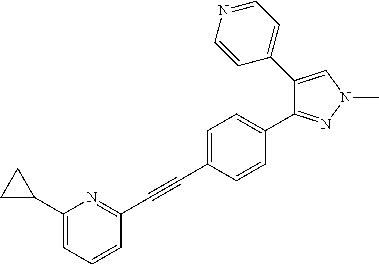 [SMILES: Cn1cc(-c2ccncc2)c(-c2ccc(C#Cc3cccc(C4CC4)n3)cc2)n1]

### Example 5A

### Preparation of 2-bromo-6-cyclopropylpyridine

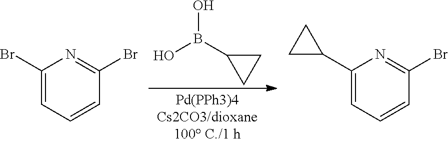

A solution of 2,6-dibromopyridine (474 mg, 2 mmol), cyclopropylboronic acid (344 mg, 4 mmol), Pd(PPh₃)₄ (116 mg, 0.1 mmol) Cs₂CO₃ (1.95 g, 6 mmol) in dioxane was degassed with N₂ at room temperature for 5 minutes, and the resulting reaction mixture was heated at 100° C. for 1 hour. the mixture was concentrated and purified by column chromatography over silica gel using (PE) to give the titled product as a clear oil (240 mg, Y: 40%); MS (ESI) m/z=198 [M+H]⁺.

### Preparation of Example 5

Example 5 was prepared using a method analogous to that of Example 1 utilizing Example 1D and 2-bromo-6-cyclopropylpyridine, as a white solid in 29% yield; ¹H NMR (400 MHz, CDCl₃) δ 8.55 (brs, 2H), 7.58-7.62 (m, 3H), 7.48-7.50 (m, 3H), 7.30-7.32 (dd, J=1.2, 7.6 Hz, 1H), 7.21 (brs, 2H), 7.02-7.04 (dd, J=1.2, 8.0 Hz, 1H), 4.02 (s, 3H), 2.07-2.13 (m, 1H); 1.00-1.07 (m, 4H); MS (ESI) m/z=377 [M+H]⁺; HPLC retention time: 1.78 minutes (Method A).

### Example 6

### 5-methyl-2-(4-(1-methyl-4-(pyridin-4-yl)-1H-pyrazol-3-yl)phenyl)ethynyl)pyridine

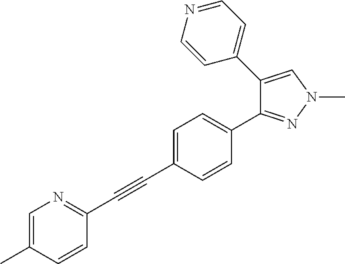

Example 6 was prepared using a method analogous to that of Example 1 utilizing Example 1D and 2-bromo-5-methylpyridine, as a white solid in 56% yield; ¹H NMR (400 MHz, CDCl₃) δ 8.54 (brs, 2H), 7.60-7.63 (m, 4H), 7.48-7.51 (m, 2H), 7.37-7.39 (d, J=8 Hz, 1H), 7.21 (brs, 2H), 7.13-7.15 (d, J=8 Hz, 1H), 4.03 (s, 3H), 2.61 (s, 3H); MS (ESI) m/z=351 [M+H]⁺, HPLC retention time: 1.84 minutes (Method A).

### Example 7

### 5-cyclopropyl-2-((4-(1-methyl-4-(pyridin-4-yl)-1H-pyrazol-3-yl)phenyl)ethynyl)pyridine

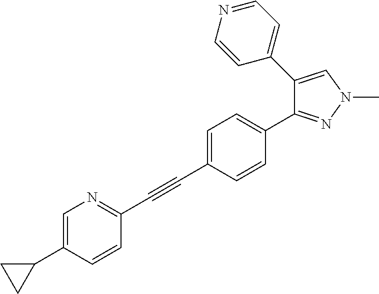 [SMILES: Cn1cc(-c2ccncc2)c(-c2ccc(C#Cc3ccc(C4CC4)cn3)cc2)n1]

### Example 7a

### Preparation of 2-chloro-5-cyclopropylpyridine

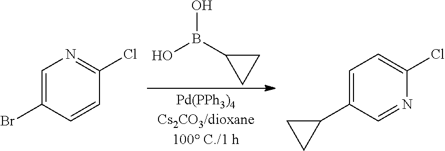

A solution of 2,6-dibromopyridine (386 mg, 2 mmol), cyclopropylboronic acid (344 mg, 4 mmol), Pd(PPh₃)₄ (116 mg, 0.1 mmol), Cs₂CO₃ (1.95 g, 6 mmol) in dioxane (10 ml) was degassed with N2 at room temperature for 5 minutes, and the resulting reaction mixture was heated at 100° C. for 1 hour. The mixture was concentrated and purified by column chromatography over silica gel using (PE) to give the titled product as a clear oil (200 mg, Y: 67%); MS (ESI) m/z=154 [M+H]⁺.

### Preparation Example 7

Example 7 was prepared using a method analogous to that of Example 1 utilizing Example 1D and 2-chloro-5-cyclopropylpyridine, as a white solid in 11% yield; ¹H NMR (400 MHz, CDCl₃) δ 8.54 (d, J=8 Hz, 2H), 8.45 (d, J=2.4 Hz, 1H), 7.58-7.62 (m, 3H), 7.43-7.50 (m, 3H), 7.30 (m, 1H), 7.21 (brs, 2H), 7.12-7.20 (m, 1H), 4.02 (s, 3H), 1.91-1.96 (m, 1H); 1.01-1.13 (m, 2H), 0.77-0.81 (m, 2H); MS (ESI) m/z=377 [M+H]⁺, HPLC retention time: 2.18 minutes.

### Example 8

### 2-((4-(1-methyl-4-(pyridin-4-yl)-1H-pyrazol-3-yl)phenyl)ethynyl)-6-(trifluoromethyl)pyridine

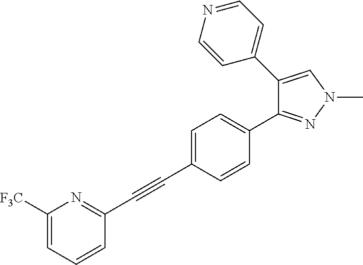 [SMILES: Cn1cc(-c2ccncc2)c(-c2ccc(C#Cc3cccc(C(F)(F)F)n3)cc2)n1]

Example 8 was prepared using a method analogous to that of Example 1 utilizing Example 1D and 2-bromo-6-trifluoromethylpyridine, as a white solid in 52% yield; ¹H NMR (400 MHz, CDCl₃) δ 8.56 (brs, 2H), 7.88-7.92 (t, 1H), 7.61-7.73 (m, 5H), 7.48-7.56 (m, 2H), 7.21 (brs, 2H), 4.03 (s, 3H); MS (ESI) m/z=405 [M+H]⁺; HPLC retention time: 2.31 minutes (Method A).

### Example 9

### 2-((4-(1-methyl-4-(pyridin-4-yl)-1H-pyrazol-3-yl)phenyl)ethynyl)-5-(trifluoromethyl)pyridine

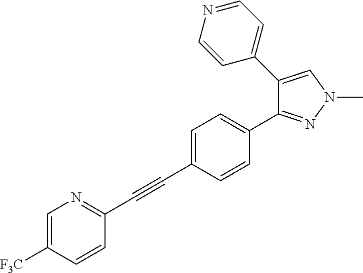 [SMILES: Cn1cc(-c2ccncc2)c(-c2ccc(C#Cc3ccc(C(F)(F)F)cn3)cc2)n1]

Example 9 was prepared using a method analogous to that of Example 1 utilizing Example 1D and 2-bromo-5-trifluoromethylpyridine, as a white solid in 57% yield; ¹H NMR (400 MHz, CDCl₃) δ 8.90 (s, 1H), 8.59 (brs, 2H), 7.94-7.96 (dd, J=2.4, 8.0 Hz, 1H), 7.62-7.67 (m, 4H), 7.52-7.54 (m, 2H), 7.24 (brs, 2H), 4.03 (s, 3H); MS (ESI) m/z=405 [M+H]⁺; HPLC retention time: 2.28 minutes (Method A).

Example 10.

### 2-methoxy-6-((4-(1-methyl-4-(pyridin-4-yl)-1H-pyrazol-3-yl)phenyl)ethynyl)pyridine

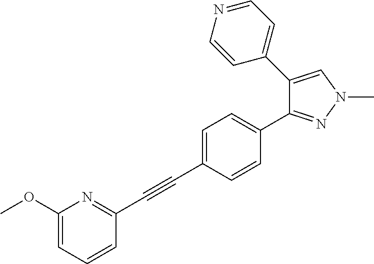

Example 8 was prepared using a method analogous to that of Example 1 utilizing Example 1D and 2-bromo-6-methoxypyridine, as a white solid in 59% yield; ¹H NMR (400 MHz, CDCl₃) δ 8.62 (brs, 1H), 7.66-7.71 (m, 1H), 7.56-7.63 (m, 4H), 7.47-7.51 (m, 3H), 7.23 (brs, 1H), 7.16-7.18 (dd, J=0.8, 7.2 Hz, 1H), 6.73-6.75 (d, J=8 Hz, 1H), 4.03 (s, 3H), 4.00 (s, 3H); MS (ESI) m/z=367 [M+H]⁺; HPLC retention time: 2.19 minutes (Method A).

### Example 11

### 2-((4-(4-(pyridin-4-yl)-1H-pyrazol-3-yl)phenyl)ethynyl)quinoline

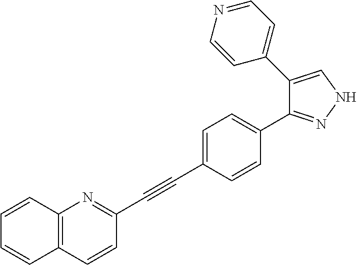 [SMILES: C(#Cc1ccc2ccccc2n1)c1ccc(-c2n[nH]cc2-c2ccncc2)cc1]

### Example 11A

### Preparation of 4-(3-(4-bromophenyl)-1H-pyrazol-4-yl)pyridine

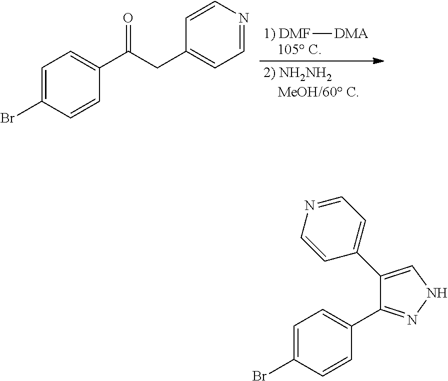

1-(4-Bromophenyl)-2-(pyridin-4-yl)ethanone (2 g, 7.2 mmol) in DMF-DMA (13 mL) was heated at reflux for 1 hour, and then concentrated. The residue was dissolved in MeOH. To this, hydrazine hydrate in water (80%) (0.3 mL) was added. The reaction mixture was heated at 65° C. for 1 hour. When LC/MS indicated the reaction was completed. The suspension was added H₂O. The product was collected, washed with EA:PE=1:10 and oven dried to give the titled product as a yellow solid (1.5 g, Y: 70%); MS (ESI) m/z=301 [M+H]⁺.

### Example 11B

### Preparation of 4-(3-(4-((trimethylsilyl)ethynyl)phenyl)-1H-pyrazol-4-yl)pyridine

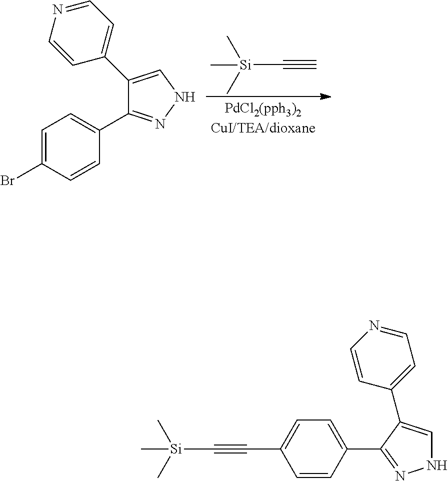

The title compound was synthesized using the procedures described in Example 1 to give the titled product as a white solid (1.2 g, Y: 95%); MS (ESI) m/z=318 [M+H]⁺.

### Example 11C

### Preparation of 4-(3-(4-ethynylphenyl)-1H-pyrazol-4-yl)pyridine

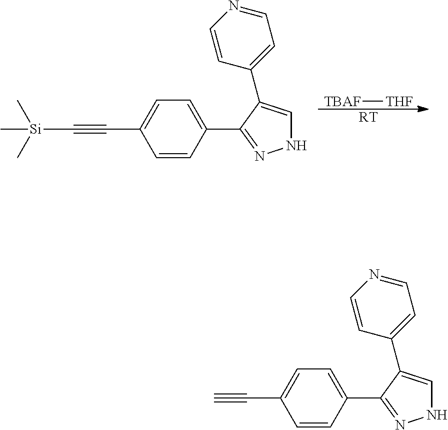

The titled compound was synthesized using the procedures described in Example 1 to give the titled product as a white solid (1.0 g, Y: 98%); MS (ESI) m/z=246 [M+H]⁺.

### Example 11

### Preparation of 2-((4-(4-(pyridin-4-yl)-1H-pyrazol-3-yl)phenyl)ethynyl)quinoline

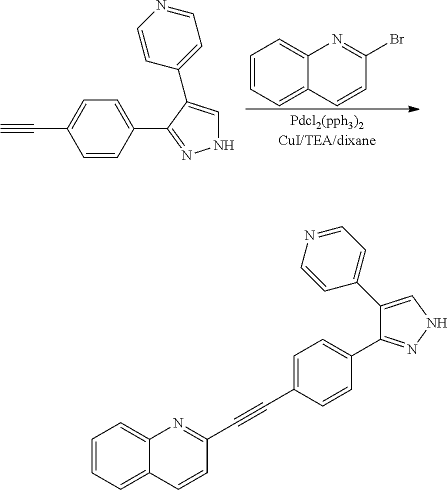

The title compound was synthesized using the procedures described in Example 1 to give the titled product as a white solid (1.3 g, Y: 87%); ¹H NMR (400 MHz, CDCl₃) δ 8.47 (brs, 2H), 8.40 (d, J=4.8 Hz, 1H), 7.98-8.05 (m, 3H), 7.65-7.86 (m, 5H), 7.56-7.58 (m, 2H), 7.40 (brs, 2H); MS (ESI) m/z=373 [M+H]⁺; HPLC retention time: 1.78 minutes (Method A).

### Example 12

### 2-((4-(1-cyclopropyl-4-(pyridin-4-yl)-1H-pyrazol-3-yl)phenyl)ethynyl)quinoline

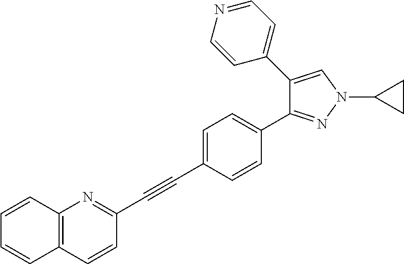 [SMILES: C(#Cc1ccc2ccccc2n1)c1ccc(-c2nn(C3CC3)cc2-c2ccncc2)cc1]

To a mixture of 2-((4-(4-(pyridin-4-yl)-1H-pyrazol-3-yl)phenyl)-ethynyl)quinoline (Example 11, 74.4 mg, 0.2 mmol), Cs₂CO₃ (65.2 mg, 0.2 mmol), cyclopropylboronic acid (68.8 mg, 0.8 mmol) in 1,4-dioxane (1.0 mL), were added DMAP (97.6 mg, 0.8 mmol), Cu(AcO)₂ (72 mg, 0.4 mmol) and 1,10-phenanthroline (144 mg, 0.8 mmol). The reaction mixture was stirred at room temperature until the complete consumption of the SM as determined by TLC and LC-MS. Then water was added and the mixture was extracted with EA three times, the combined organic layers were washed with brine and dried (anhydrous Na₂SO₄). After filtration and concentration, the residue was purified by prep-HPLC to give the desired product as a yellow solid (30.4 mg, yield: 37%); LC/MS: m/z [M⁺1]=413; ¹H NMR (400 M, CDCl₃) δ 8.60 (brs, 2H), 8.14 (t, J=8.4 Hz, 2H), 7.81 (dd, J=8.4, 1.2 Hz, 1H), 7.78-7.74 (m, 1H), 7.72-7.62 (m, 4H), 7.61-7.56 (m, 1H), 7.54-7.50 (m, 2H), 7.25 (brs, 2H), 3.75-3.60 (m, 1H), 1.30-1.24 (m, 2H), 1.17-1.10 (m, 2H); HPLC retention time: 2.13 minutes (Method A).

### Example 13

### 2-(4-(pyridin-4-yl)-3-(4-(quinolin-2-ylethynyl)phenyl)-1H-pyrazol-1-yl)ethanol

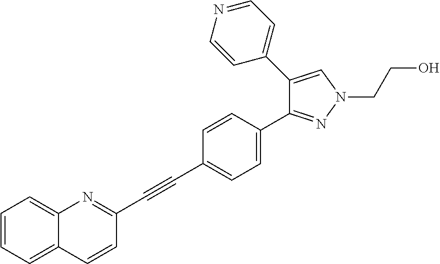 [SMILES: OCCn1cc(-c2ccncc2)c(-c2ccc(C#Cc3ccc4ccccc4n3)cc2)n1]

To a mixture of 2-((4-(4-(pyridin-4-yl)-1H-pyrazol-3-yl)phenyl)-ethynyl)quinoline (Example 11, 150 mg, 0.4 mmol) and Cs₂CO₃ (652 mg, 2.0 mmol) in DMF (2.0 mL) at room temperature, was added 1,3-dioxolan-2-one (176 mg, 2.0 mmol). The reaction mixture was stirred at this temperature until the complete consumption of the SM as determined by TLC and LC-MS. Then water was added and the mixture was extracted with EA three times, the combined organic layers were washed with brine and dried (anhydrous Na₂SO₄). After filtration and concentration, the residue was purified by prep-HPLC to give the desired product as a yellow solid (56.2 mg, yield: 27%). LC/MS: m/z [M⁺+1]=417; ¹H NMR (400 M, CDCl₃) δ 8.49 (s, 2H), 8.20-8.10 (m, 2H), 7.81 (d, J=7.6 Hz, 1H), 7.77-7.68 (m, 2H), 7.65-7.60 (m, 3H), 7.59-7.53 (m, 1H), 7.52-7.45 (m, 2H), 7.17 (d, J=5.6 Hz, 2H), 4.34 (t, J=4.8 Hz, 2H), 4.12 (t, J=4.8 Hz, 2H), 1.38 (brs, 1H); HPLC retention time: 1.76 min (Method A).

### Example 14

### N-methyl-2-(4-(pyridin-4-yl)-3-(4-(quinolin-2-ylethynyl)phenyl)-1H-pyrazol-1-yl)ethanamine

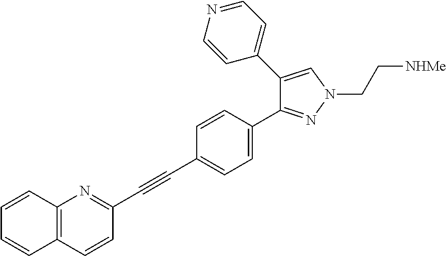 [SMILES: CNCCn1cc(-c2ccncc2)c(-c2ccc(C#Cc3ccc4ccccc4n3)cc2)n1]

To a mixture of 2-(4-(pyridin-4-yl)-3-(4-(quinolin-2-ylethynyl)phenyl)-1H-pyrazol-1-yl)ethanol (Example 13, 28.1 mg, 0.067 mmol) in THF (2.0 mL) at room temperature, two drops of TEA (about 20.2 mg, 0.2 mmol) and one drop of MsCl (11.4 mg, 0.1 mmol) were added, and the reaction was stirred at this temperature till the complete consumption of the SM as determined by TLC and LC-MS. Then methylamine in THF (4 mL) was added, to ensure the pH of the mixture≧10. The reaction was then heated at 100° C. in a seal tube. After stirred for 48 hours, the reaction was cooled down, water was added. The mixture was extracted with EA three times, the combined organic layers were washed with brine and dried (anhydrous Na₂SO₄). After filtration and concentration, the residue was purified by prep-HPLC to give the desired product as a yellow solid (21.8 mg, yield: 25%). LC/MS: m/z [M⁺1]=430; ¹H NMR (400 M, CDCl₃) δ 8.49 (s, 2H), 8.35 (brs, 1H), 8.18-8.08 (m, 2H), 7.80 (d, J=8.4 Hz, 1H), 7.77-7.70 (m, 2H), 7.67-7.52 (m, 4H), 7.50-7.40 (m, 2H), 7.30-7.20 (m, 2H), 4.51 (s, 2H), 3.44 (s, 2H), 2.62 (s, 3H); HPLC retention time: 1.40 minutes (Method A).

### Example 15

### 2-((4-(1-ethyl-4-(pyridin-4-yl)-1H-pyrazol-3-yl)phenyl)ethynyl)quinoline

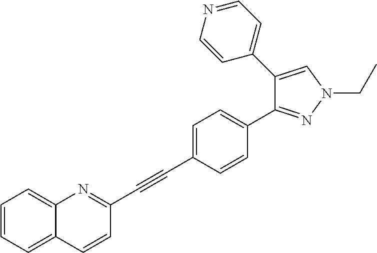 [SMILES: CCn1cc(-c2ccncc2)c(-c2ccc(C#Cc3ccc4ccccc4n3)cc2)n1]

To a suspension of 2-((4-(4-(pyridin-4-yl)-1H-pyrazol-3-yl)phenyl)ethynyl)quinoline (Example 11, 70 mg, 0.19 mmol) and Cs₂CO₃ (185 mg, 0.57 mmol) in DMF (1 mL). was added bromoethane (41 mg, 0.38 mmol) at room temperature. The mixture was stirred at room temperature for 15 minutes and then diluted with EA and washed with brine. The organic layer was then dried over N₂SO₄, filtered and concentrated. The residue was purified by prep-TLC to give the desired product as a yellow solid (30 mg, Y: 40%); ¹H NMR (400 MHz, CDCl₃) δ 8.54 (brs, 2H), 8.14-8.19 (t, 21H), 7.83-7.85 (d, J=8 Hz, 1H), 7.74-7.76 (m, 1H), 7.63-7.68 (m, 4H), 7.53-7.60 (m, 3H), 7.21-7.23 (brs, 2H), 4.26-4.32 (q, 2H), 1.59-1.63 (t, 3H); MS (ESI) m/z=401 [M+H]⁺; HPLC retention time: 2.1 minutes (Method A).

### Example 16

### 2-((4-(1-(2-methoxyethyl)-4-(pyridin-4-yl)-1H-pyrazol-3-yl)phenyl)ethynyl)quinoline

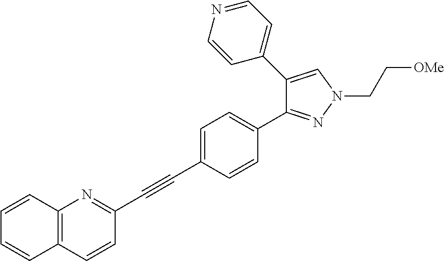 [SMILES: COCCn1cc(-c2ccncc2)c(-c2ccc(C#Cc3ccc4ccccc4n3)cc2)n1]

Example 16 was prepared using a method analogous to that of Example 15 utilizing Example 11 and 1-bromo-2-methoxyethane, as a yellow solid in 41% yield after prep-HPLC purification; LC/MS: m/z [M⁺+1]=431; ¹H NMR (400 M, CDCl₃) δ 8.52 (brs, 2H), 8.14 (t, J=8.8 Hz, 2H), 7.81 (d, J=7.6 Hz, 1H), 7.75-7.67 (m, 2H), 7.66-7.60 (m, 3H), 7.55-7.45 (m, 3H), 7.21 (brs, 2H), 4.37 (t, J=5.2 Hz, 2H), 3.83 (t, J=5.2 Hz, 2H), 3.39 (s, 3H); HPLC retention time: 1.99 minutes (Method A).

### Example 17

### N,N-dimethyl-2-(4-(pyridine-4-yl)-3-(4-(quinolin-2-ylethynyl)phenyl)-1H-pyrazol-1-yl)ethanamine

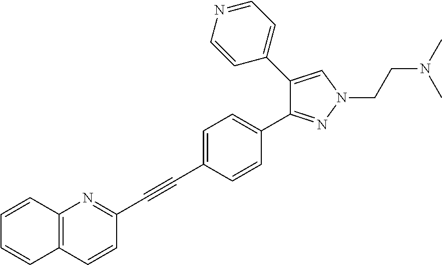 [SMILES: CN(C)CCn1cc(-c2ccncc2)c(-c2ccc(C#Cc3ccc4ccccc4n3)cc2)n1]

Example 17 was prepared using a method analogous to that of Example 15 utilizing Example 12 and 2-chloro-N,N-dimethylethanamine hydrochloride, as a yellow solid in 24% yield after prep-HPLC purification; LC/MS: m/z [M⁺1]=444. ¹H NMR (400 M, CDCl₃) δ 8.59 (brs, 2 H), 8.14 (t, J=8.8 Hz, 2H), 7.83 (d, J=8.0 Hz, 1H), 7.80-7.70 (m, 2H), 7.70-7.60 (m, 3H), 7.58 (t, J=7.2 Hz, 1H), 7.53 (d, J=7.6 Hz, 2H), 7.25 (s, 2H), 4.43 (t, J=6.0 Hz, 4H), 2.43 (s, 6 H); HPLC retention time: 2.30 minutes (Method A).

### Example 18

### 2-((4-4-(pyridin-4-yl)-1-(2,2,2-trifluoroethyl)1H-pyrazol-3-yl)phenyl)ethynyl)quinoline

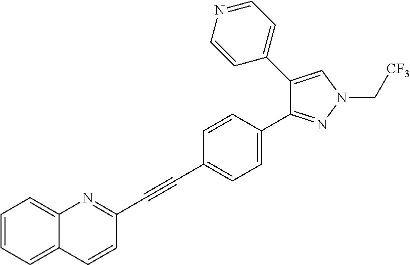 [SMILES: FC(F)(F)Cn1cc(-c2ccncc2)c(-c2ccc(C#Cc3ccc4ccccc4n3)cc2)n1]

Example 18 was prepared using a method analogous to that of Example 15 utilizing Example 11 and 1,1,1-trifluoro-2-iodoethane, as a yellow solid in 50% yield after prep-HPLC purification; LC/MS: m/z [M⁺1]=455; ¹H NMR (400 M, CDCl₃) δ 8.56 (brs, 2H), 8.20-8.10 (m, 2H), 7.82 (d, J=8.0 Hz, 1H), 7.80-7.70 (m, 2H), 7.69-7.60 (m, 3H), 7.59-7.52 (m, 1H), 7.50-7.45 (m, 2H), 7.23-7.10 (m, 2H), −5.00-4.90 (m, 2H); HPLC retention time: 2.33 minutes (Method A).

### Example 19

### 2-((4-(1-(2-fluoroethyl)-4-(pyridin-4-yl)-1H-pyrazol-3-yl)phenyl)ethynyl)quinoline

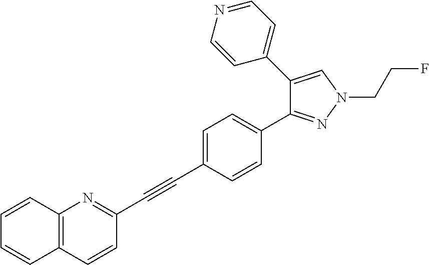 [SMILES: FCCn1cc(-c2ccncc2)c(-c2ccc(C#Cc3ccc4ccccc4n3)cc2)n1]

Example 19 was prepared using a method analogous to that of Example 15 utilizing Example 11 and 1-bromo-2-fluoroethane, as a yellow solid in 29% yield after prep-HPLC purification; LC/MS: m/z [M⁺+1]=419; ¹H NMR (400 M, CDCl₃) δ 8.55 (brs, 1H), 8.25-8.10 (m, 2H), 8.00-7.50 (m, 10H), 7.26 (brs, 2H), 5.00-4.70 (m, 2H), 4.60-4.40 (m, 2H); HPLC retention time: 2.03 minutes (Method A).

### Example 20

### 2-((4-(5-(pyridin-4-yl)pyrimidin-4-yl)phenyl)ethynyl)quinoline

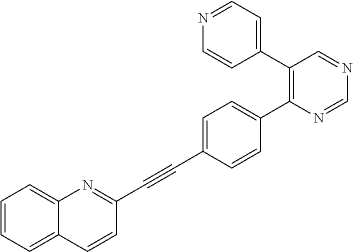 [SMILES: C(#Cc1ccc2ccccc2n1)c1ccc(-c2ncncc2-c2ccncc2)cc1]

### Example 20A

### Preparation of 4-(4-bromophenyl)-5-(pyridin-4-yl)pyrimidine

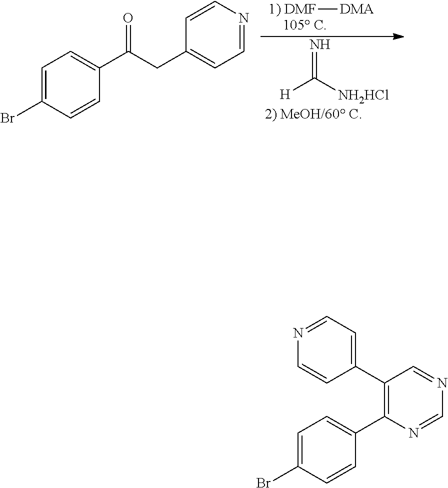

A mixture of 1-(4-Bromophenyl)-2-(pyridin-4-yl)ethanone (500 mg, 1.8 mmol) in DMF-DMA (2 mL) was heated at reflux for 1 hour and then concentrated. The residue was dissolved in EtOH. To this, formimidamide hydrochloride (288 mg, 3.6 mmol) was added. In a separate flask sodium (100 mg) was added to EtOH (8 mL) and stirred for 10 minutes. The EtONa solution was added to the reaction mixture and the resulting mixture was heated at reflux for 1 hour. The mixture was concentrated and purified by column chromatography over silica gel using (PE:EA=1:1) to give the desired product as a white solid (150 mg, Y: 27%); MS (ESI) m/z=313 [M+H]⁺.

### Example 20B

### Preparation of 5-(pyridin-4-yl)-4-(4-((trimethylsilyl)ethynyl)phenyl)pyrimidine

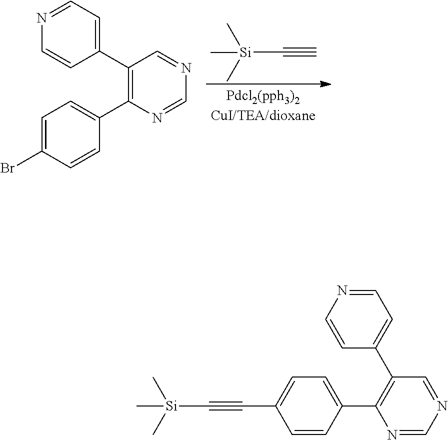

The titled compound was synthesized using the procedures described in Example 1 to give the desired product as a yellow oil (110 mg, Y: 95%); MS (ESI) m/z=330 [M+H]⁺.

### Example 20C

### Preparation of 4-(4-ethynylphenyl)-5-(pyridin-4-yl)pyrimidine

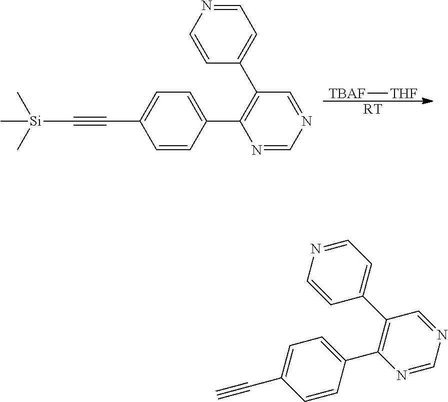

The titled compound was synthesized using the procedures described in Example 1 to give the desired product as a yellow solid (100 mg, Y: 95%); MS (ESI) m/z=258 [M+H]⁺.

### Example 20

### Ppreparation of 2-((4-(5-(pyridin-4-yl)pyrimidin-4-yl)phenyl)ethynyl)quinoline

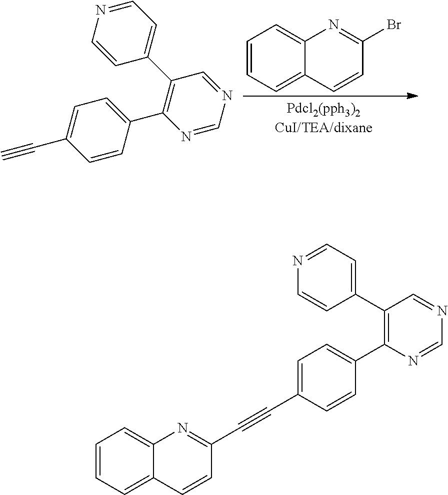

The titled example was prepared using a method analogous to that of Example 1 utilizing Example 20C and 2-bromoquinoline, as a yellow solid in 68% yield; ¹H NMR (400 MHz, CDCl₃) δ 9.36 (s, 1H), 8.79 (s, 1H), 8.67 (brs, 2H), 8.14-8.20 (m, 2H), 7.75-7.86 (m, 2H), 7.59-7.56 (m, 4H), 7.48-7.50 (m, 2H), 7.20-7.22 (d, J=8 Hz, 2H); MS (ESI) m/z=385 [M+H]⁺; HPLC retention time: 2.34 minutes (Method A).

### Example 21

### 2-((4-(5-(pyridin-4-yl)-1H-1,2,4-triazol-1-yl)phenyl)ethynyl)quinoline

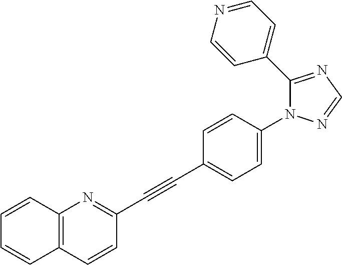 [SMILES: C(#Cc1ccc2ccccc2n1)c1ccc(-n2ncnc2-c2ccncc2)cc1]

### Example 21A

### Preparation of N-((dimethylamino)methylene)isonicotinamide

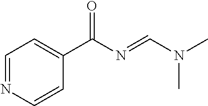

Under N₂, isonicotinamide (2.44 g, 20 mmol) was dissolved in DMF-DMA (20 mL). The reaction mixture was refluxed for 1 hour as judged by TLC and LC-MS. After removing the solvent, washed with hexane and dried to afford an orange solid which was used for the next step without further purification (3.4 g, 96% yield); LC/MS: m/z [M⁺+1]=178.

### Example 21B

### Preparation of 4-(1-(4-bromophenyl)-1H-1,2,4-triazol-5-yl)pyridine

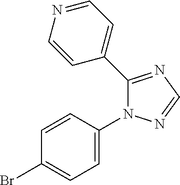 [SMILES: Brc1ccc(-n2ncnc2-c2ccncc2)cc1]

To a solution of 4-beomophenylhydrazine hydrochloride (1.34 g, 6.0 mmol) in acetic acid (10 mL) and 1,4-dioxane (5 mL), N-((dimethylamino)methylene)isonicotinamide (0.89 g, 5.0 mmol) was added. The reaction mixture was then stirred at 90° C. for 3 hours till the complete consumption of the N-((dimethylamino)methylene)isonicotinamide as determined by TLC and LC-MS. After cooled to room temperature, a yellow solid was obtained which was washed with n-hexane (1.5 g, 99% yield); LC/MS: m/z [M⁺+1]=301.

### Example 21C

### Preparation of 4-(1-(4-((trimethylsilyl)ethynyl)phenyl)-1H-1,2,4-triazol-5-yl)pyridine

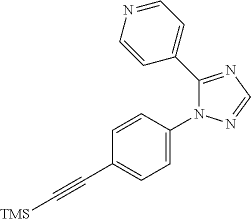 [SMILES: C[Si](C)(C)C#Cc1ccc(-n2ncnc2-c2ccncc2)cc1]

A mixture of 4-(1-(4-bromophenyl)-1H-1,2,4-triazol-5-yl)pyridine (602 mg, 2.0 mmol), PdCl₂(PPh₃)₂ (70.2 mg, 5 mol %), CuI (19.1 mg, 5 mol %) in TEA (10 mL) and 1,4-dioxane (5 mL) was bubbled with dry N₂ for 5 minutes. Then ethynyltrimethylsilane (588 mg, 6 mmol) was added and this reaction mixture was refluxed at 100° C. until the complete consumption of the SM as determined by TLC and LC-MS. Then water was added and the mixture was extracted with EA three times, the combined organic layers were washed with brine and dried (anhydrous Na₂SO₄). After filtration and concentration, the residue was purified by flash column chromatography on silica gel to give the desired product as a yellow solid (0.54 g, 85% yield. LC/MS: m/z [M⁺+1]=319.

### Example 21D

### Preparation of 4-(1-(4-ethynylphenyl)-1H-1,2,4-triazol-5-yl)pyridine

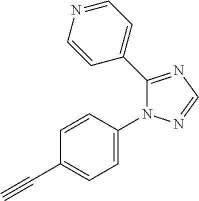 [SMILES: C#Cc1ccc(-n2ncnc2-c2ccncc2)cc1]

At room temperature, to 4-(1-(4-((trimethylsilyl)ethynyl)phenyl)-1H-1,2,4-triazol-5-yl)pyridine (0.54 g, 1.7 mmol) was added TBAF (2 mL, 1 M in THF). The reaction was stirred at room temperature till the complete consumption of the SM as determined by TLC and LC-MS. Then water was added and the mixture was extracted with EA three times, the combined organic layers were washed with brine and dried (anhydrous Na₂SO₄). After filtration and concentration, the residue was purified by flash column chromatography on silica gel to give the desired product as a yellow solid (387 mg, 93% yield). LC/MS: m/z [M⁺1]=247; ¹H NMR (400 M, CDCl₃) δ 8.70-8.60 (m, 2H), 8.14 (s, 1H), 7.57 (d, J=8.4 Hz, 2H), 7.37 (d, J=5.6 Hz, 2H), 7.31 (d, J=8.4 Hz, 2H), 3.20 (s, 1H).

### Example 21

### Synthesis of 2-((4-(5-(pyridin-4-yl)-1H-1,2,4-triazol-1-yl)phenyl)ethynyl)quinoline

A mixture of 4-(1-(4-ethynylphenyl)-1H-1,2,4-triazol-5-yl)pyridine (Example 21D, 73.8 mg, 0.3 mmol), PdCl₂(PPh₃)₂ (10.5 mg, 5 mol %), CuI (2.9 mg, 5 mol %) in TEA (3 mL) and 1,4-dioxane (1.5 mL) was bubbled with dry N₂ for 5 minutes. Then 2-bromoquinoline (62.4 mg, 0.3 mmol) was added. The resulting reaction mixture was refluxed at 100° C. until the complete consumption of the SM as determined by TLC and LC-MS. Then water was added and the mixture was extracted with EA three times, the combined organic layers were washed with brine and dried (anhydrous Na₂SO₄). After filtration and concentration, the residue was purified by flash column chromatography on silica gel to give the desired product (86.1 mg) as a yellow solid in 77% yield. LC/MS: m/z [M⁺+1]=374; ¹H NMR (400 M, CDCl₃) δ 8.67 (d, J=5.6 Hz, 2H), 8.21-8.10 (m, 3H), 7.85-7.80 (m, 1H), 7.80-7.73 (m, 3H), 7.65-7.55 (m, 2H), 7.45-7.35 (m, 4H); HPLC retention time: 2.42 minutes (Method A).

### Example 22

### 2-((4-(5-(pyridin-4-yl)-1H-pyrazol-1-yl)phenyl)ethynyl)quinoline

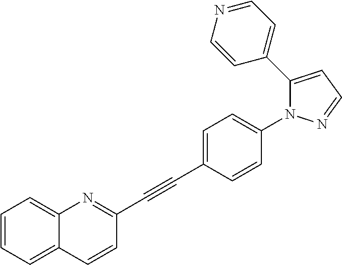 [SMILES: C(#Cc1ccc2ccccc2n1)c1ccc(-n2nccc2-c2ccncc2)cc1]

### Example 22A

### Preparation of 4-(1-(4-bromophenyl)-1H-pyrazol-5-yl)pyridine

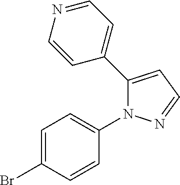 [SMILES: Brc1ccc(-n2nccc2-c2ccncc2)cc1]

A mixture of 4-acetylpyridine (904 mg, 7.46 mmol) and DMF-DMA (4.5 mL) was heated to 105° C. for 1 hour. The resulting solution was cooled and concentrated. Then 4-bromophenylhydrazine hydrochloride (97%, 1.72 g, 7.46 mmol), acetic acid (1.1 mL, 19.20 mmol) and methanol (20 mL) were added. The solution was stirred at 60° C. for 15 hour. After cooling, the mixture was poured into 40 mL of saturated NaHCO₃ aqueous solution and extracted with ethyl acetate (50 mL). The organic layer was separated and the aqueous layer was extracted with ethyl acetate (30 mL). The combined organic layers were washed with brine (30 mL), dried over Na₂SO₄, filtered and concentrated to give the crude product which was purified by column chromatograph (PE:EA 10:1-8:1-5:1) to give the product (1.28 g, yield: 57.1%) as an orange solid. LC/MS: m/z M⁺+1=300, 302. ¹H NMR (400 MHz, CDCl₃) δ 8.58-8.60 (m, 2H), 7.78 (d, J=2.0 Hz, 1H), 7.51-7.55 (m, 2H), 7.18-7.21 (m, 2H), 7.13-7.15 (m, 2H), 6.65 (d, J=1.6 Hz, 1H).

### Example 22B

### Preparation of 4-(1-(4-((trimethylsilanyl)ethynyl)phenyl)-1H-pyrazol-5-yl)pyridine

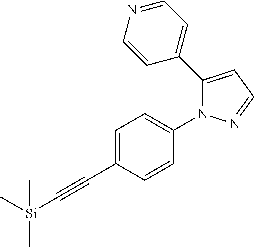 [SMILES: C[Si](C)(C)C#Cc1ccc(-n2nccc2-c2ccncc2)cc1]

To a mixture of 4-(1-(4-bromophenyl)-1H-pyrazol-5-yl)pyrimidine (900 mg, 3.0 mmol), PdCl₂(PPh₃)₂ (84.2 mg, 0.12 mmol) and CuI (22.8 mg, 0.12 mmol) in Et₃N/dioxane (8 mL/4 mL) was bubbled N₂ for 15 minutes. Then ethynyltrimethylsilane (1.47 g, 15.0 mmol) was added and the resulting mixture was stirred at 100° C. for 1 hour under N₂ protection. After cooling, the mixture was poured into 20 mL of cooled water and extracted with ethyl acetate (3×30 mL). The combined organic layers were washed with brine (30 mL), dried over Na₂SO₄, filtered and concentrated to give the crude product which was purified by column chromatography (PE:EA 10:1-8:1) to give the product (720 mg, yield: 75.6%) as a yellow solid. LC/MS: m/z M⁺1=318; ¹H NMR (400 MHz, CDCl₃) δ 8.58 (d, J=4.0 Hz, 2H), 7.78-7.79 (m, 1H), 7.48-7.51 (m, 2H), 7.25-7.28 (m, 2H), 7.13-7.14 (m, 2H), 6.66 (d, J=2.0 Hz, 1H), 0.28 (s, 9H).

### Example 22C

### Preparation of 4-(1-(4-ethynylphenyl)-1H-pyrazol-5-yl)pyridine

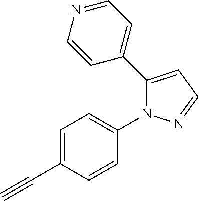 [SMILES: C#Cc1ccc(-n2nccc2-c2ccncc2)cc1]

A mixture of 4-(1-(4-((trimethylsilanyl)ethynyl)phenyl)-1H-pyrazol-5-yl)pyridine (720 mg, 2.27 mmol) in TBAF (1M in THF, 8 mL) was stirred at 25° C. for 2 hours. Then water (20 mL) and ethyl acetate (30 mL) were added. The organic layer was separated and the aqueous layer was extracted with ethyl acetate (25 mL). The combined organic layers were washed with brine (20 mL), dried over Na₂SO₄, filtered and concentrated to give the crude product which was purified by column chromatography (PE:EA 10:1-3:1) to give the product (460 mg, yield: 82.6%) as a light yellow solid. LC/MS: m/z M⁺1=246. ¹H NMR (400 MHz, CDCl₃) δ 8.60 (d, J=6.0 Hz, 2H), 7.79 (d, J=2.0 Hz, 1H), 7.52-7.54 (m, 2H), 7.28-7.30 (m, 2H), 7.14-7.16 (m, 2H), 6.66 (d, J=1.6 Hz 1H), 3.17 (s, 1H).

### Example 22

### Preparation of 2-((4-(5-(pyridin-4-yl)-1H-pyrazol-1-yl)phenyl)ethynyl)quinoline

In a 25 mL of three-necked flask, 2-bromoquinoline (130 mg, 0.625 mmol), PdCl₂(PPh₃)₂ (17 mg, 0.024 mmol) and CuI (4.6 mg, 0.024 mmol) were mixed in Et₃N/dioxane (4 mL/2 mL) and the mixture was bubbled N₂ for 10 minutes. Then 4-(1-(4-ethynylphenyl)-1H-pyrazol-5-yl)pyridine (150 mg, 0.615 mmol) dissolved in 2 mL of 1,4-dioxane was added and the resulting mixture was bubbled to N₂ for 15 min. Then the mixture was stirred at 100° C. for 1 hours under N₂ protection. After cooling, the mixture was poured into 20 mL, of cooled water and extracted with ethyl acetate (3×20 mL). The combined organic layers were washed with brine (30 mL), dried over Na₂SO₄, filtered and concentrated to give the crude product which was purified by column chromatograph (PE:EA 5:1-1:1) to give the product (130 mg, yield: 57.0%) as a yellow solid, LC/MS: m/z M⁺1=373; HPLC retention time=2.85 minutes (Method B); ¹H NMR (400 MHz, CDCl₃) δ 8.58 (d, J=5.2 Hz, 2H), 8.14-8.20 (q, 2H), 7.75-7.86 (m, 3H), 7.69-7.71 (m, 2H), 7.58-7.65 (m, 2H), 7.45 (d, J=8 Hz, 2H), 7.18 (d, J=8 Hz, 2H), 6.68 (d, J=1.6 Hz, 1H).

### Example 23

### 2-((4-(3-(pyridin-4-yl)-4H-1,2,4-triazol-4-yl)phenyl)ethynyl)quinoline

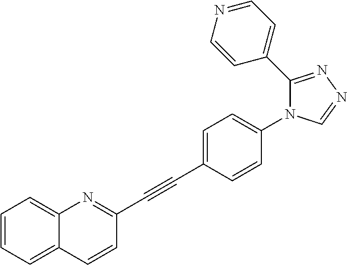 [SMILES: C(#Cc1ccc2ccccc2n1)c1ccc(-n2cnnc2-c2ccncc2)cc1]

### Example 23A

### Preparation of N′-Isonictinoyl-N,N-dimethylhydrazonoformamide

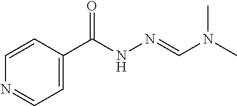

DMF-DMA (3.2 mL, 24.1 mmol) was added dropwise to a stirred solution of isoniazid (2.7 g, 19.7 mmol) in acetonitrile (230 mL) at 50° C. The reaction mixture was stirred at the same temperature for 10 minutes. The solvent was removed under vacuum and the residue was washed with diethyl ether (100 mL). The solid was filtered and dried to give the product (3.3 g, yield: 87.1%). LC/MS: m/z M⁺+1=193.

### Example 23B

### Preparation of 4-(4-(4-bromophenyl)-4H-1,2,4-triazol-3-yl)pyridine

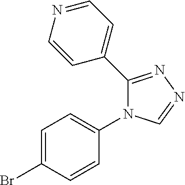 [SMILES: Brc1ccc(-n2cnnc2-c2ccncc2)cc1]

To a mixture of N′-isonictinoyl-N,N-dimethylhydrazonoformamide (2.94 g, 3.0 mmol) in acetic acid (90 mL), was added 4-bromoaniline (97%, 2.71 g, 15.3 mmol). The mixture was stirred at 120° C. for 3 hours under N₂ protection. After cooling, the mixture was concentrated. Ethyl acetate (20 mL) was added and the mixture was stirred for 10 minutes. White solid was precipitated, filtered and washed with ethyl acetate (10 mL). Then the solid was partitioned between ethyl acetate (100 mL) and 15% NaOH aqueous solution. The aqueous layer was separated and extracted with ethyl acetate (80 mL). The combined organic layers were dried over Na₂SO₄, filtered and concentrated to give the product (1.5 g, yield: 32.5%) as a white solid. LC/MS: m/z M⁺+1=301, 303; ¹H NMR (400 MHz, CDCl₃) δ 8.66-8.67 (m, 2H), 8.39 (s, 1H), 7.70 (d, J=4.4 Hz, 2H), 7.39-7.41 (m, 2H), 7.17-7.19 (m, 2H), 7.18 (d, J=8.8 Hz, 2H).

### Example 23C

### Preparation of 4-(4-(4-((trimethylsilyl)ethynyl)phenyl)-4H-1,2,4-triazol-3-yl)pyridine

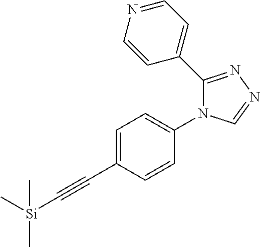 [SMILES: C[Si](C)(C)C#Cc1ccc(-n2cnnc2-c2ccncc2)cc1]

To a mixture of 4-(4-(4-bromophenyl)-4H-1,2,4-triazol-3-yl)pyridine (900 mg, 3.0 mmol), PdCl₂(PPh₃)₂ (84.2 mg, 0.12 mmol) and CuI (22.8 mg, 0.12 mmol) in Et₃N/dioxane (8 mL/4 mL) was bubbled N₂ for 15 minutes. Then ethynyltrimethylsilane (1.47 g, 15.0 mmol) was added and the resulting mixture was stirred at 100° C. for 1 hour under N₂ protection. After cooling, the mixture was poured into 20 mL of cooled water and extracted with ethyl acetate (3×30 mL). The combined organic layers were washed with brine (30 mL), dried over Na₂SO₄, filtered and concentrated to give the crude product which was purified by column chromatography (PE:EA 10:1-8:1) to give the product (750 mg, yield: 78.6%) as a yellow solid. LC/MS: m/z M⁺1=319.

### Example 23D

### Preparation of 4-(4-(4-ethynylphenyl)-4H-1,2,4-triazol-3-yl)pyridine

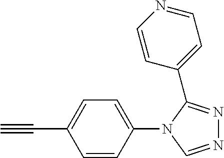 [SMILES: C#Cc1ccc(-n2cnnc2-c2ccncc2)cc1]

A solution of 4-(1-(4-((trimethylsilanyl)ethynyl)phenyl)-1H-pyrazol-5-yl)pyridine (750 mg, 2.35 mmol) dissolved in TBAF-THF (1M, 8 mL) was stirred at 25° C. for 2 hours. Then water (20 mL) and ethyl acetate (30 mL) were added. The organic layer was separated and the aqueous layer was extracted with ethyl acetate (25 mL). The combined organic layers were washed with brine (20 mL), dried over Na₂SO₄, filtered and concentrated to give the crude product which was purified by column chromatography (PE:EA 10:1-3:1) to give the product (480 mg, yield: 82.8%) as a light yellow solid. LC/MS: m/z M⁺1=247.

### Example 23

### Preparation of 2-((4-(3-(pyridin-4-yl)-4H-1,2,4-triazol-4-yl)phenyl)ethynyl)quinoline

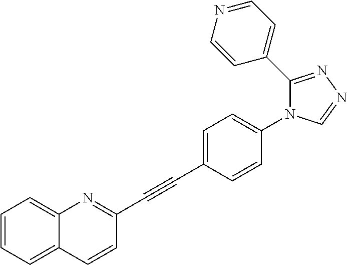 [SMILES: C(#Cc1ccc2ccccc2n1)c1ccc(-n2cnnc2-c2ccncc2)cc1]

In a 25 mL of three-necked flask, 2-bromoquinoline (130 mg, 0.625 mmol), PdCl₂(PPh₃)₂ (17 mg, 0.024 mmol) and CuI (4.6 mg, 0.024 mmol) were mixed in Et₃N/dioxane (4 mL/2 mL) and the mixture was bubbled N₂ for 10 minutes. Then 4-(1-(4-ethynylphenyl)-1H-pyrazol-5-yl)pyridine (151 mg, 0.615 mmol) dissolved in 2 mL of 1,4-dioxane was added and the resulting mixture was bubbled to N₂ for 15 minutes. Then the mixture was stirred at 100° C. for 1 hour under N₂ protection. After cooling, the mixture was poured into 20 mL of cooled water and extracted with ethyl acetate (3×20 mL). The combined organic layers were washed with brine (30 mL), dried over Na₂SO₄, filtered and concentrated to give the crude product which was purified by column chromatograph (PE:EA 5:1-1:1) to give the product (130 mg, yield: 57.0%) as a yellow solid, LC/MS: m/z M⁺1=374; HPLC retention time=2.85 minutes (Method B); ¹H NMR (400 MHz, CDCl₃) δ 8.66 (d, J=5.6 Hz, 2H), 8.45 (s, 1H), 8.22 (d, J=8.8 Hz, 1H), 8.16 (d, J=8.8 Hz, 1H), 7.78-7.88 (m, 4H), 7.60-7.67 (m, 2H), 7.41 (d, J=5.6 Hz, 2H), 7.31 (d, J=8.4 Hz, 2H).

### Example 24

### 2-(5-(1-methyl-4-pyridin-4-yl-1H-pyrazol-3-yl)pyridin-2-ylethynyl)quinoline

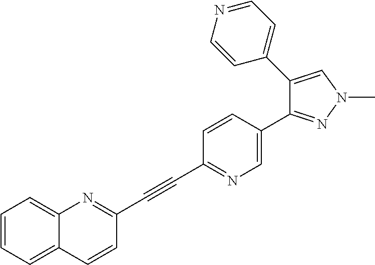 [SMILES: Cn1cc(-c2ccncc2)c(-c2ccc(C#Cc3ccc4ccccc4n3)nc2)n1]

### Example 24A

### Preparation of 6-bromo-N-methoxy-N-methylnicotinamide

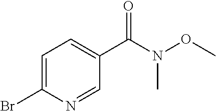

To a mixture of 6-bromonictinic acid (2.0 g, 9.9 mmol), N,O-dimethyl hydroxyl amine hydrochloride (1.16 g, 11.9 mmol) and Et₃N (1.66 mL, 11.9 mmol) in dry CH₂Cl₂ (20 mL), was added EDCI (2.28 g, 11.9 mmol) in portions at 0° C. The mixture was then stirred at 25° C. for 16 hours and washed with water (20 mL). The organic layer was separated and the aqueous layer was extracted with CH₂Cl₂ (20 mL). The combined organic layers were washed with brine (20 mL), dried over MgSO₄, filtered and concentrated to give the product (2.36 g, yield: 97%) as yellow oil which was used directly for the next step without further purification, LC/MS: m/z M⁺+1=245, 247; ¹H NMR (400 MHz, CDCl₃) δ 8.74 (d, J=2.4 Hz, 1H), 7.90-7.93 (m, 1H), 7.56 (d, J=8.4 Hz, 1H), 3.56 (s, 3H), 3.39 (s, 3H).

### Example 24B

### Preparation of 1-(6-bromopyridin-3-yl)-2-(pyridin-4-yl)ethanone

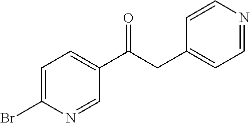 [SMILES: O=C(Cc1ccncc1)c1ccc(Br)nc1]

To a solution of 4-picoline (2.7 g, 29.0 mmol) in dry THF (15 mL), was added LDA (2.0 M in THF, 14.4 mL, 28.8 mmol) dropwise at 0° C. After the addition, the mixture was stirred at 0° C. for 40 minutes. In a separated round bottom flask, 6-bromo-N-methoxy-N-methylnicotinamide (2.36 g, 9.6 mmol) was dissolved in 20 mL of dry THF and cooled to −60° C. Then 1.5 eq. of the 4-picoline anion was added dropwise. After 45 minutes, the remaining 4-picoline anion was added dropwise. Then the reaction mixture was stirred at −60° C. for 1 hour and quenched by addition of NH₄Cl (aq., 20 mL). Then water (15 mL) and ethyl acetate (20 mL) were added. The organic layer was separated and the aqueous layer was extracted with ethyl acetate (30 mL). The combined organic layers were washed with brine (30 mL), dried over Na₂SO₄, filtered and concentrated. Crude product was precipitated. The precipitate was filtered and washed with petroleum ether (25 mL) to give the crude product (1.6 g, yield: 60.1%) as a yellow solid which was used for next step without further purification. LC/MS: m/z M⁺1=277, 279.

### Example 24C

### Preparation of 2-bromo-5-(1-methyl-4-(pyridin-4-yl)-1H-pyrazol-3-yl)pyridine

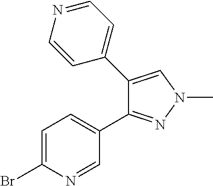 [SMILES: Cn1cc(-c2ccncc2)c(-c2ccc(Br)nc2)n1]

A mixture of 1-(6-bromopyridin-3-yl)-2-(pyridin-4-yl)ethanone (1.6 g, 5.77 mmol) and DMF-DMA (8 mL) was heated to 105° C. and stirred for 1 hour. The resulting solution was cooled and concentrated to give the intermediate which was dissolved in 40% H₂NNHCH₃ (aq., 2.0 g, 17.36 mmol) and methanol (8 mL). The solution was stirred at 65° C. for 1.5 hours. After cooling, the mixture was concentrated to give the crude product which was purified by column chromatography (PE:EA 8:1-5:1-2:1) to give the product (major isomer) (560 mg, yield: 30.8%) as a yellow solid. LC/MS: m/z M⁺1=315, 317; ¹H NMR (400 MHz, CDCl₃) δ 8.50-8.57 (m, 3H), 7.63-7.66 (m, 2H), 7.47-7.49 (m, 1H), 7.15-7.17 (m, 2H), 4.02 (s, 3H).

### Example 24D

### Preparation of 5-1-methyl-4-(pyridin-4-yl)-1H-pyrazol-3-yl)-2-((trimethylsilanyl)ethynyl)pyridine

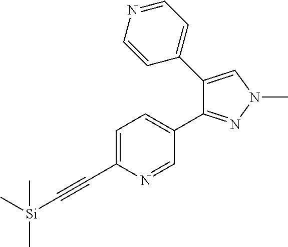

To a mixture of 2-bromo-5-(1-methyl-4-(pyridin-4-yl)-1H-pyrazol-3-yl)pyridine (160 mg, 0.51 mmol), PdCl₂(PPh₃)₂ (14.3 mg, 0.02 mmol) and CuI (3.9 mg, 0.02 mmol) in Et₃N/dioxane (4 mL/2 mL) was bubbled N₂ for 15 minutes. Then ethynyltrimethylsilane (250 mg, 2.55 mmol) was added, and the resulting mixture was stirred at 100° C. for 1 hour under N₂. After cooling, the mixture was poured into 20 mL of cooled water and extracted with ethyl acetate (3×30 mL). The combined organic layers were washed with brine (25 mL), dried over Na₂SO₄, filtered and concentrated to give the crude product which was purified by column chromatography (PE:EA 5:1-2:1) to give 280 mg of the crude product as yellow oil which was used for next step directly without further purification. LC/MS: m/z M⁺1=333; ¹H NMR (400 MHz, CDCl₃) δ 8.69-8.70 (m, 1H), 8.51-8.53 (m, 2H), 7.75 (dd, J=8.0 Hz, 2.4 Hz, 1H), 7.62 (s, 1H), 7.45 (dd, J=8.0 Hz, 0.8 Hz, 1H), 7.14-7.15 (m, 2H), 4.02 (s, 3H), 0.28 (s, 9H).

### Example 24E

### Preparation of 2-ethynyl-5-(1-methyl-4-(pyridin-4-yl)-1H-pyrazol-3-yl)pyridine

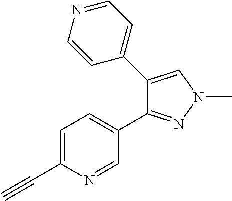 [SMILES: C#Cc1ccc(-c2nn(C)cc2-c2ccncc2)cn1]

A solution of the crude mixture from the former step in TBAF-THF (4 mL) was stirred at 25° C. for 2 hours. Then the mixture was concentrated and purified by column chromatography (PE:EA 5:1-3:1-1:1) to give the product (70 mg, yield: 52.7% for two steps) as a light yellow solid. LC/MS: m/z M⁺1=261; ¹H NMR (400 MHz, CDCl₃) δ 8.74 (d, J=2.0 Hz, 1H), 8.55 (d, J=6.0 Hz, 2H), 7.78 (dd, J=8.0 Hz, 2.0 Hz, 1H), 7.64 (s, 1H), 7.48 (d, J=8.0 Hz, 1H), 7.16-7.18 (m, 2H), 4.04 (s, 3H), 3.23 (s, 1H).

### Example 24

### Preparation of 2-((5-(1-methyl-4-(pyridin-4-yl)-1H-pyrazol-3-yl)pyridin-2-yl)ethynyl)quinoline

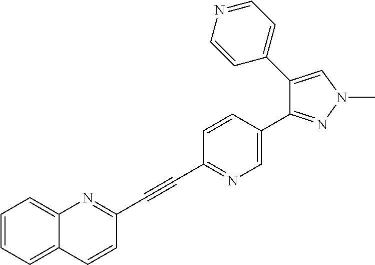 [SMILES: Cn1cc(-c2ccncc2)c(-c2ccc(C#Cc3ccc4ccccc4n3)nc2)n1]

A mixture of 2-bromoquinoline (56 mg, 0.27 mmol), PdCl₂(PPh₃)₂ (7.6 mg, 0.011 mmol) and CuI (2.1 mg, 0.011 mmol) in Et₃N/dioxane (4 mL/2 mL) was bubbled N₂ for 10 minutes. Then 2-ethynyl-5-(1-methyl-4-(pyridin-4-yl)-1H-pyrazol-3-yl)pyridine (70 mg, 0.27 mmol) dissolved in 2 mL of 1,4-dioxane was added, and the resulting mixture was bubbled to N₂ for 10 minutes. Then the mixture was stirred at 100° C. for 1.5 hours under N₂ protection. After cooling, the mixture was poured into 20 mL of cooled water and extracted with ethyl acetate (5×30 mL). The combined organic layers were washed with brine (30 mL), dried over Na₂SO₄, filtered and concentrated to give the crude product which was purified by column chromatograph (DCM:MeOH 100:1-60:1) to give the crude product which was then purified by prep. HPLC to give the product (28.5 mg, yield: 27.4%) as a yellow solid, LC/MS: m/z M⁺+1=388; HPLC retention time=2.22 minutes (Method B); ¹H NMR (400 MHz, CDCl₃) δ 8.81-8.82 (m, 1H), 8.57-8.58 (m, 2H), 8.15-8.21 (m, 2H), 7.85-7.87 (m, 2H), 7.69-7.80 (m, 3H), 7.65 (s, 1H), 7.58-7.62 (m, 1H), 7.20-7.21 (m, 2H), 4.06 (s, 3H).

### Example 25

### 2-((3-fluoro-4-(1-methyl-4-(pyridin-4-yl)-1H-pyrazol-3-yl)phenyl)ethynyl)quinoline

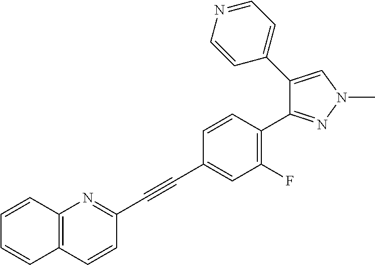 [SMILES: Cn1cc(-c2ccncc2)c(-c2ccc(C#Cc3ccc4ccccc4n3)cc2F)n1]

### Example 25A

### Preparation of 4-bromo-2-fluoro-N-methoxy-N-methylbenzamide

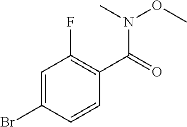

At 0° C., to the solution of 4-bromo-2-fluorobenzoic acid (2.19 g, 10.0 mmol), N,O-dimethylhydroxylamine hydrochloride (1.17 g, 12 mmol) and TEA (1.7 mL, 12 mmol), was added EDCI (2.3 g, 12 mmol) in batches (control the temperature below 5° C.). After addition, the reaction was allowed to warm to room temperature, until the complete consumption of the SM as determined by TLC and LC-MS. Then water was added and the mixture was extracted with EA three times, the combined organic layers were washed with brine and dried (anhydrous Na₂SO₄). After filtration and concentration, the desired product was obtained (2.6 g) as a yellow solid in 99% yield. LC/MS: m/z [M⁺+1]=262; ¹H NMR (400 M, CDCl₃) δ 7.46-7.20 (m, 3H), 3.54 (s, 3 H), 3.34 (s, 3H).

### Example 25B

### Preparation of 1-(4-bromo-2-fluorophenyl)-2-(pyridin-4-yl)ethanone

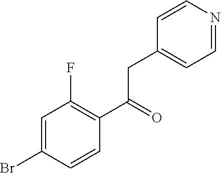 [SMILES: O=C(Cc1ccncc1)c1ccc(Br)cc1F]

At 0° C., to a solution of 4-methylpyridine (1.1 mL, 11.5 mmol), was added dropwise LDA (5.7 mL, 11.5 mmol). After addition, the reaction mixture was stirred for another 0.5 hour at this temperature. At the same time, 4-bromo-2-fluoro-N-methoxy-N-methylbenzamide (1.0 g, 3.8 mmol) was dissolved in THF and cooled to −65° C. The formed 4-methylpyridine lithium salt was added one third, after stirred for 45 minutes, the remaining was added, and the reaction was monitored by TLC and LC-MS. After the complete consumption of the SM, saturated NH₄Cl was added and the mixture was extracted with EA three times. The combined organic layers were washed with brine and dried (anhydrous Na₂SO₄). After filtration and concentration, the desired product was obtained (0.93 g) as a yellow solid in 83% yield. LC/MS: m/z [M⁺+1]=294; ¹H NMR (400 M, CDCl₃) δ 8.54 (d, J=6.0 Hz, 2H), 7.75 (d, J=8.0 Hz, 1H), 7.42-7.37 (m, 2H), 7.20-7.17 (m, 2H), 4.24 (d, J=2.8 Hz, 2H).

### Example 25C

### Preparation of 4-(3-(4-bromo-2-fluorophenyl)-1-methyl-1H-pyrazol-4-yl)pyridine

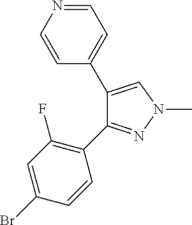 [SMILES: Cn1cc(-c2ccncc2)c(-c2ccc(Br)cc2F)n1]

A solution of 1-(4-bromo-2-fluorophenyl)-2-(pyridin-4-yl)ethanone (0.89 g, 3.0 mmol) in DMF-DMA (3 mL) was refluxed for 1.0 hour at 105° C. then cooled down and the solvent was removed to afford the intermediate without purification. Then this intermediate was dissolved in MeOH (5 mL). To this solution, methylhydrazine (0.91 g, 7.8 mmol) was added and refluxed till the complete consumption of the intermediate as determined by TLC and LC-MS. After removing the solvent, the residue was purified by flash column chromatography on silica gel to give the desired product (0.7 g) as a yellow solid in 70% yield. LC/MS: m/z [M⁺+1]=332; ¹H NMR (400 M, CDCl₃) δ 8.48 (d, J=5.6 Hz, 2H), 7.89 (s, 1H), 7.40-7.35 (m, 2H), 7.30-7.26 (m, 1H), 7.10-7.05 (m, 2H), 4.02 (s, 3H).

### Example 25D

### Preparation of 4-(3-(2-fluoro-4-((trimethylsilyl)ethynyl)phenyl)-1-methyl-1H-pyrazol-4-yl)pyridine

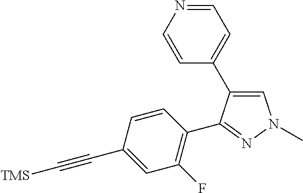 [SMILES: Cn1cc(-c2ccncc2)c(-c2ccc(C#C[Si](C)(C)C)cc2F)n1]

A mixture of 4-(3-(4-bromo-2-fluorophenyl)-1-methyl-1H-pyrazol-4-yl)pyridine (100 mg, 0.3 mmol), PdCl₂(PPh₃)₂ (10.5 mg, 5 mol %), CuI (2.9 mg, 5 mol %) in TEA (2 mL) and 1,4-dioxane (1.0 mL) was bubbled with dry N₂ for 5 minutes. Then ethynyltrimethylsilane (88.6 mg, 0.9 mmol) was added and this reaction mixture was refluxed at 100° C. until the complete consumption of the SM as determined by TLC and LC-MS. Then water was added and the mixture was extracted with EA three times, the combined organic layers were washed with brine and dried (anhydrous Na₂SO₄). After filtration and concentration, the residue was purified by flash column chromatography on silica gel to give the desired product (74.9 mg) as a yellow solid in 72% yield. LC/MS: m/z [M⁺+1]=350.

### Example 25E

### Preparation of 4-(3-(4-ethynyl-2-fluorophenyl)-1-methyl-1H-pyrazol-4-yl)pyridine

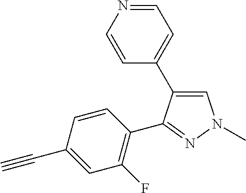 [SMILES: C#Cc1ccc(-c2nn(C)cc2-c2ccncc2)c(F)c1]

A mixture of 4-(3-(2-fluoro-4-((trimethylsilyl)ethynyl)phenyl)-1-methyl-1H-pyrazol-4-yl)pyridine (70 mg, 0.2 mmol) in TBAF (1 mL, 1 M in THF) was stirred at room temperature till the complete consumption of the SM as determined by TLC and LC-MS. Then water was added and the mixture was extracted with EA three times, the combined organic layers were washed with brine and dried (anhydrous Na₂SO₄). After filtration and concentration, the residue was purified by flash column chromatography on silica gel to give the desired product (54 mg) as a light yellow solid in 99% yield. LC/MS: m/z [M⁺+1]=278.

### Example 25

### Synthesis of 2-((3-fluoro-4-(1-methyl-4-(pyridin-4-yl)-1H-pyrazol-3-yl)phenyl)ethynyl)quinoline

A mixture of 4-(3-(4-ethynyl-2-fluorophenyl)-1-methyl-1H-pyrazol-4-yl)pyridine (54 mg, 0.2 mmol), PdCl₂(PPh₃)₂ (7.0 mg, 5 mol %), CuI (1.9 mg, 5 mol %) in TEA (2 mL) and 1,4-dioxane (1 mL) was bubbled with dry N₂ for 5 minutes. Then 2-bromoquinoline (40.5 mg, 0.2 mmol) was added and this reaction mixture was refluxed at 100° C. till the complete consumption of the SM as determined by TLC and LC-MS. Then water was added and the mixture was extracted with EA three times, the combined organic layers were washed with brine and dried (anhydrous Na₂SO₄). After filtration and concentration, the residue was purified by pre-HPLC to give the desired product (11.5 mg) as a light yellow solid in 14% yield. LC/MS: m/z [M⁺+1]=405; ¹H NMR (400 M, CDCl₃) δ 8.51 (s, 2H), 8.20-8.10 (m, 2H), 7.83 (d, J=8.0 Hz, 1H), 7.77-7.73 (m, 1H), 7.70 (s, 1H), 7.62 (d, J=8.0 Hz, 2H), 7.59-7.54 (m, 1H), 7.53-7.50 (m, 2H), 7.38 (d, J=11.4 Hz, 2H), 7.13 (brs, 2H), 4.03 (s, 3H); HPLC retention: 2.10 minutes (Method B).

### Example 26

### 2-(4-(1-methyl-4-pyridin-4-yl-1H-pyrazol-3-yl)phenylethynyl)quinoline

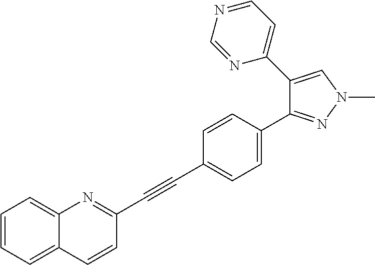 [SMILES: Cn1cc(-c2ccncn2)c(-c2ccc(C#Cc3ccc4ccccc4n3)cc2)n1]

### Example 26A

### Preparation of 4-bromo-N-methoxy-N-methylbenzamide

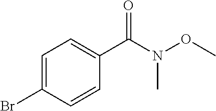

To a mixture of 4-bromobenzoic acid (30.0 g, 149.2 mmol), N,O-dimethyl hydroxyl amine hydrochloride (18.0 g, 184.5 mmol) and Et₃N (26 mL, 186.5 mmol) in dry CH₂Cl₂ (300 mL), was added EDCI (35.3 g, 184.1 mmol) in portions at 0° C. The mixture was then stirred at 30° C. for 2 hours and washed with water (300 mL). The organic layer was separated and the aqueous layer was extracted with CH₂Cl₂ (150 mL). The combined organic layers were washed with brine (200 mL), dried over MgSO₄, filtered and concentrated to give the product (36.0 g, yield: 98.9%) as yellow oil which was used directly for the next step without further purification, LC/MS: m/z M⁺1=244, 246; ¹H NMR (400 MHz, CDCl₃) δ 7.53-7.60 (m, 4H), 3.54 (s, 3H), 3.36 (s, 3H).

### Example 26B

### Preparation of 1-(4-bromophenyl)-2-(pyrimidin-4-yl)ethanone

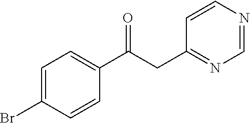 [SMILES: O=C(Cc1ccncn1)c1ccc(Br)cc1]

To a solution of 4-methylpyrimidine (2.2 g, 23.4 mmol) in dry THF (10 mL), was added LDA (2.0 M in THF, 11.6 mL, 23.2 mmol) dropwise at 0° C. After the addition, the mixture was stirred at 0° C. for 40 minutes. In a separated round bottom flask, 4-bromo-N-methoxy-N-methylbenzamide (1.88 g, 7.7 mmol) was dissolved in 20 mL of dry THF and cooled to −65° C. Then 1.5 eq. of the anion solution was added dropwise. After 30 minutes, the remaining anion was added dropwise. Then the reaction mixture was stirred at −65° C. for 2 hours and quenched by addition of NH₄Cl (aq., 20 mL). Then water (15 mL) and ethyl acetate (20 mL) were added. The organic layer was separated and the aqueous layer was extracted with ethyl acetate (30 mL). The combined organic layers were washed with brine (30 mL), dried over Na₂SO₄, filtered and concentrated to give the crude product which was purified by column chromatography (PE:EA 10:1-5:1-3:1) to give the product (1.5 g, yield: 70.4%) as a yellow solid. LC/MS: m/z M⁺1=277, 279.

### Example 26C

### Preparation of 4-(3-(4-bromophenyl)-1-methyl-1H-pyrazol-4-yl)pyrimidine

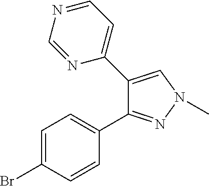 [SMILES: Cn1cc(-c2ccncn2)c(-c2ccc(Br)cc2)n1]

A mixture of 1-(4-bromophenyl)-2-(pyrimidin-4-yl)ethanone (1.5 g, 5.4 mmol) in DMF-DMA (8 mL) was heated to 105° C. and stirred for 1 hour. The resulting solution was cooled and concentrated to give the intermediate which was dissolved in 40% H₂NNHCH₃ (aq., 1.9 g, 16.5 mmol) and methanol (10 mL). The solution was stirred at 65° C. for 1.5 hours. After cooling, the mixture was concentrated to give the crude product which was purified by column chromatography (PE:EA 5:1-3:1-2:1) to give the product (major isomer) (880 mg, yield: 51.8%) as a light yellow solid. LC/MS: m/z M⁺1=315, 317; ¹H NMR (400 MHz, CDCl₃) δ 9.07 (d, J=1.6 Hz, 1H), 8.44 (d, J=5.6 Hz, 1H), 8.22 (s, 1H), 7.69-7.72 (m, 2H), 7.26-7.29 (m, 2H), 6.85 (dd, J=5.6 Hz, 1.6 Hz, 1H), 3.77 (s, 3H).

### Example 26D

### Preparation of 4-(1-methyl-3-(4-((trimethylsilanyl)ethynyl)phenyl)-1H-pyrazol-4-yl)pyrimidine

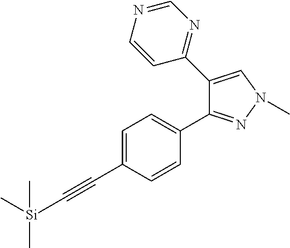

A mixture of 4-(3-(4-bromophenyl)-1-methyl-1H-pyrazol-4-yl)pyrimidine (500 mg, 1.59 mmol), PdCl₂(PPh₃)₂ (44.5 mg, 0.063 mmol) and CuI (11.5 mg, 0.060 mmol) in Et₃N/dioxane (6 mL/3 mL) was bubbled N₂ for 15 minutes. Then ethynyltrimethylsilane (780 mg, 7.94 mmol) was added and the resulting mixture was stirred at 100° C. for 1 hour under N₂ protection. After cooling, the mixture was poured into 20 mL of cooled water and extracted with ethyl acetate (3×30 mL). The combined organic layers were washed with brine (30 mL), dried over Na₂SO₄, filtered and concentrated to give the crude product which was purified by column chromatography (PE:EA 5:1-2:1) to give the product (440 mg, yield: 83.2%) as a light yellow solid. LC/MS: m/z M⁺+1=333; ¹H NMR (400 MHz, CDCl₃) δ 9.07 (s, 1H), 8.39 (d, J=5.2 Hz, 1H), 8.23 (s, 1H), 7.64-7.66 (m, 2H), 7.29-7.34 (m, 2H), 6.78-6.80 (m, 1H), 3.75 (s, 3H), 0.30 (s, 9H).

### Example 26E

### Preparation of 4-(3-(4-ethynylphenyl)-1-methyl-1H-pyrazol-4-yl)pyrimidine

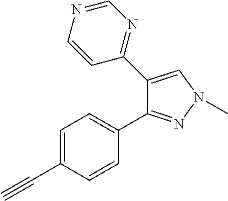 [SMILES: C#Cc1ccc(-c2nn(C)cc2-c2ccncn2)cc1]

A solution of 4-(1-methyl-3-(4-((trimethylsilanyl)ethynyl)phenyl)-1H-pyrazol-4-yl)pyrimidine (440 mg, 1.32 mmol) in TBAF-THF (1M, 6 mL) was stirred at 25° C. for 2 hours. Then water (15 mL) and ethyl acetate (30 mL) were added. The organic layer was separated and the aqueous layer was extracted with ethyl acetate (3×15 mL). The combined organic layers were washed with brine (20 mL), dried over Na₂SO₄, filtered and concentrated to give the crude product which was purified by column chromatography (PE:EA 10:1-2:1) to give the product (328 mg, yield: 95.4%) as a light yellow solid. LC/MS: m/z M⁺+1=261; ¹H NMR (400 MHz, CDCl₃) δ 9.07 (s, 1H), 8.42 (d, J=4.8 Hz, 1H), 8.22 (s, 1H), 7.67-7.69 (m, 2H), 7.35-7.37 (m, 2H), 6.81-6.83 (m, 1H), 3.77 (s, 3H), 3.24 (s, 1H).

### Example 26

### Preparation of 2-(4-(1-methyl-4-pyridin-4-yl-1H-pyrazol-3-yl)phenylethynyl)quinoline

In a 25 mL of three-necked flask, 2-bromoquinoline (130 mg, 0.625 mmol), PdCl₂(PPh₃)₂ (17 mg, 0.024 mmol) and CuI (4.6 mg, 0.024 mmol) were mixed in Et₃N/dioxane (4 mL/2 mL) and the mixture was bubbled N₂ for 10 minutes. Then 4-(3-(4-ethynylphenyl)-1-methyl-1H-pyrazol-4-yl)pyrimidine (160 mg, 0.615 mmol) dissolved in 2 mL of 1,4-dioxane was added and the resulting mixture was bubbled to N₂ for 15 minutes. Then the mixture was stirred at 100° C. for 1 hour under N₂ protection. After cooling, the mixture was poured into 20 mL of cooled water and extracted with ethyl acetate (3×20 mL). The combined organic layers were washed with brine (30 mL), dried over Na₂SO₄, filtered and concentrated to give the crude product which was purified by column chromatograph (PE:EA 10:1-2:1) to give the product (143 mg, yield: 60.1%) as a yellow solid, LC/MS: m/z M⁺+1=389; HPLC retention time=2.85 minutes (Method B); ¹H NMR (400 MHz, CDCl₃) δ 9.09 (s, 1H), 8.44 (d, J=5.2 Hz, 1H), 8.16-8.24 (m, 3H), 7.77-7.87 (m, 4H), 7.59-7.68 (m, 2H), 7.43 (d, J=8.0 Hz, 2H), 6.84-6.86 (m, 1H), 3.81 (s, 3H).

### Example 27

### 2-((6-(1-methyl-4-(pyridin-4-yl)-1H-pyrazol-3-yl)pyridin-3-yl)ethynyl)quinoline

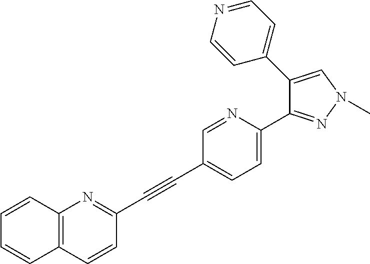 [SMILES: Cn1cc(-c2ccncc2)c(-c2ccc(C#Cc3ccc4ccccc4n3)cn2)n1]

### Example 27A

### Preparation of 5-bromo-N-methoxy-N-methylpicolinamide

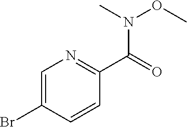

At 0° C., to the solution of 5-bromopicolinic acid (2.02 g, 10.0 mmol), N,O-dimethylhydroxylamine hydrochloride (1.17 g, 12 mmol) and TEA (1.7 mL, 12 mmol) in DCM (20 mL), was added EDCI (2.3 g, 12 mmol) in batches (temp. kept below 5° C.). After addition, the reaction mixture was allowed to warm to room temperature, till the complete consumption of the SM as determined by TLC and LC-MS. Then water was added and the mixture was extracted with DCM three times. The combined organic extracts were washed with brine and dried (anhydrous Na₂SO₄). After filtration and concentration, the desired product was obtained as a yellow solid (1.8 g, 73% yield). LC/MS: m/z [M⁺+1]=245.

### Example 27B

### Preparation of 1-(5-bromopyridin-2-yl)-2-(pyridin-4-yl)ethanone

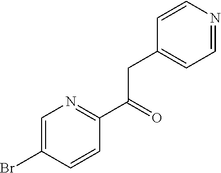 [SMILES: O=C(Cc1ccncc1)c1ccc(Br)cn1]

To a solution of 4-methylpyridine (2.2 mL, 22 mmol) at 0° C., was added dropwise LDA (11.5 mL, 22 mmol). The reaction mixture was stirred for another 0.5 hour at this temperature. At the same time, 5-bromo-N-methoxy-N-methyl picolinamide (1.8 g, 7.35 mmol) was dissolved in THF and cooled to −65° C. The formed 4-methylpyridine lithium salt was added one third, after stirred for 45 minutes, the left was added and the reaction was determined by TLC and LC-MS. After the complete consumption of the SM, saturated NH₄Cl was added and the mixture was extracted with EA three times. The combined organic extracts were washed with brine and dried (anhydrous Na₂SO₄). After filtration and concentration, the desired product was obtained as a yellow solid (1.1 g, 55% yield); LC/MS: m/z [M⁺+1]=277.

### Example 27C

### Preparation of 5-bromo-2-(1-methyl-4-(pyridin-4-yl)-1H-pyrazol-3-yl)pyridine

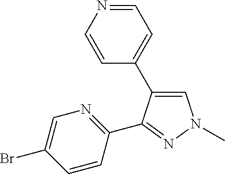 [SMILES: Cn1cc(-c2ccncc2)c(-c2ccc(Br)cn2)n1]

A mixture of 1-(5-bromopyridin-2-yl)-2-(pyridin-4-yl)ethanone (0.5 g, 1.8 mmol) in DMF-DMA (5 mL) was refluxed for 1 hour at 105° C. Then the mixture was cooled down and concentrated to afford the intermediate, which was dissolved in MeOH (4 mL). To this solution, methylhydrazine (0.91 g, 7.8 mmol) was added and the resulting mixture was refluxed till the complete consumption of the intermediate as determined by TLC and LC-MS. After concentration, the residue was purified by flash column chromatography on silica gel to give the desired product as a yellow solid (0.23 g, 40% yield). LC/MS: m/z [M⁺+1]=315; ¹H NMR (400 M, CDCl₃) δ 8.64 (d, J=2.0 Hz, 1H), 8.54 (d, J=5.6 Hz, 2H), 7.83-7.79 (m, 1H), 7.61 (s, 1H), 7.52 (d, J=7.6 Hz, 1H), 7.28-7.26 (m, 1H), 4.03 (s, 3H).

### Example 27D

### Preparation of 2-(1-methyl-4-(pyridin-4-yl)-1H-pyrazol-3-yl)-5-((trimethylsilyl)ethynyl)pyridine

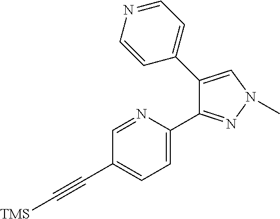 [SMILES: Cn1cc(-c2ccncc2)c(-c2ccc(C#C[Si](C)(C)C)cn2)n1]

A mixture of 5-bromo-2-(1-methyl-4-(pyridin-4-yl)-1H-pyrazol-3-yl)pyridine (90 mg, 0.29 mmol), PdCl₂(PPh₃)₂ (10.1 mg, 5 mol %), and CuI (2.6 mg, 5 mol %) in TEA (2 mL) and 1,4-dioxane (1.0 mL) was bubbled with dry N₂ for 5 minutes at room temperature. Then ethynyltrimethylsilane (85.3 mg, 0.87 mmol) was added and this reaction mixture was refluxed at 100° C. till the complete consumption of the SM as determined by TLC and LC-MS. Then water was added and the mixture was extracted with EA three times, the combined organic extracts were washed with brine and dried (anhydrous Na₂SO₄). After filtration and concentration, the residue was purified by flash column chromatography on silica gel to give the desired product as a yellow solid (83.6 mg, 88% yield. LC/MS: m/z [M⁺+1]=333.

### Example 27E

### Preparation of 5-ethynyl-2-(1-methyl-4-(pyridin-4-yl)-1H-pyrazol-3-yl)pyridine

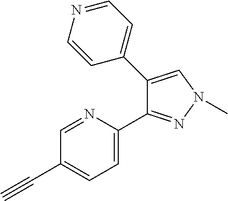 [SMILES: C#Cc1ccc(-c2nn(C)cc2-c2ccncc2)nc1]

A mixture of 2-(1-methyl-4-(pyridin-4-yl)-1H-pyrazol-3-yl)-5-((trimethylsilyl)ethynyl)pyridine (83.6 mg, 0.25 mmol) and TBAF (0.75 mL, 1 M in THF) was stirred at room temperature till the complete consumption of the SM as determined by TLC and LC-MS. Then water was added and the mixture was extracted with EA three times. The combined organic extracts were washed with brine and dried (anhydrous Na₂SO₄). After filtration and concentration, the residue was purified by flash column chromatography on silica gel to give the desired product as a light yellow solid (58.6 mg, 90% yield. LC/MS: m/z [M⁺+1]=261.

### Example 27

### Synthesis of 2-((6-(1-methyl-4-(pyridin-4-yl)-1H-pyrazol-3-yl)pyridin-3-yl)ethynyl)quinoline

A mixture of 5-ethynyl-2-(1-methyl-4-(pyridin-4-yl)-1H-pyrazol-3-yl)pyridine (52 mg, 0.2 mmol), PdCl₂(PPh₃)₂ (7.0 mg, 5 mol %), and CuI (1.9 mg, 5 mol %) in TEA (2 mL) and 1,4-dioxane (1 mL) was bubbled with dry N₂ for 5 minutes. Then 2-bromoquinoline (40.5 mg, 0.2 mmol) was added and this reaction mixture was refluxed at 100° C. till the complete consumption of the SM as determined by TLC and LC-MS. Then water was added and the mixture was extracted with EA three times, the combined organic layers were washed with brine and dried (anhydrous Na₂SO₄). After filtration and concentration, the residue was purified by flash column chromatography on silica gel to give the desired product as a light yellow solid (55.7 mg, 72% yield). LC/MS: m/z [M⁺+1]=388; HPLC retention time=2.09 min (Method A).

¹H NMR (400 M, CDCl₃) δ 8.86 (d, J=1.6 Hz, 1H), 8.54 (s, 2H), 8.17 (d, J=8.4 Hz, 1H), 8.13 (d, J=8.4 Hz, 1H), 7.94-7.90 (m, 1H), 7.82 (d, J=8.0 Hz, 1H), 7.78-7.71 (m, 1H), 7.65-7.55 (m, 4H), 7.29 (brs, 2H), 4.03 (s, 3H).

### Example 28

### 2-((4-(1-(pyridin-4-yl)-1H-pyrazol-5-yl)phenyl)ethynyl)quinoline

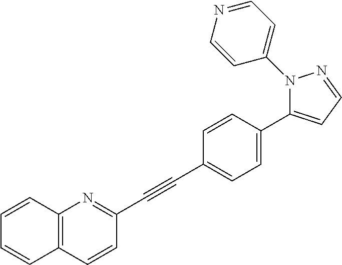 [SMILES: C(#Cc1ccc2ccccc2n1)c1ccc(-c2ccnn2-c2ccncc2)cc1]

### Example 28A

### Preparation of 4-hydrazinylpyridine

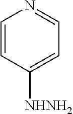

A mixture of 4-chloropyridine hydrochloride (3.0 g, 20 mmol) in hydrazine hydrate (6 mL) was refluxed for 2 hours. After removing the solvent, the titled compound was obtained (2.1 g, 96% yield), which was used for next step reaction without further purification. LC/MS: m/z [M⁺1]=110.

### Example 28B

### Preparation of 1-(4-bromophenyl)-3-(dimethylamino)prop-2-en-1-one

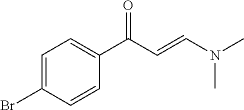

1-(4-bromophenyl)ethanone (4.97 g, 25 mmol) was dissolved in in 20 mL DMF-DMA, and refluxed for 1 hour. After the solvent was removed, the TM was obtained (5.58 g, 88% yield), which can be used without further purification. LC/MS: m/z [M⁺+1]=254.

¹H NMR (400 M, CDCl₃) δ 7.80-7.70 (m, 3H), 7.53-7.47 (m, 2H), 5.62 (d, J=12.0 Hz, 1H), 3.11 (s, 3H), 2.88 (s, 3H).

### Example 28C

### Preparation of 4-(5-(4-bromophenyl)-1H-pyrazol-1-yl)pyridine

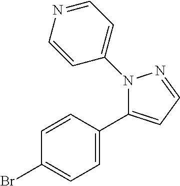 [SMILES: Brc1ccc(-c2ccnn2-c2ccncc2)cc1]

A mixture of 1-(4-bromophenyl)-3-(dimethylamino)prop-2-en-1-one (1.27 g, 5 mmol) and 4-hydrazinylpyridine (1.09 g, 10 mmol) in AcOH (30 mL) was refluxed for 3 hours. The mixture was concentrated to afford the titled compound (0.94 g, 63% yield). LC/MS: m/z [M⁺+1]=300. ¹H NMR (400 M, CDCl₃) δ 8.55 (d, J=6.4 Hz, 2H), 7.75 (d, J=2.0 Hz, 1H), 7.52-7.48 (m, 2H), 7.24-7.21 (m, 2H), 7.15-7.10 (m, 2H), 6.50 (d, J=2.0 Hz, 1H)

### Example 28D

### Preparation of 4-(5-(4-((trimethylsilyl)ethynyl)phenyl)-1H-pyrazol-1-yl)pyridine

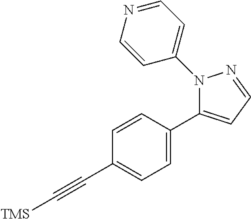 [SMILES: C[Si](C)(C)C#Cc1ccc(-c2ccnn2-c2ccncc2)cc1]

A mixture of 4-(5-(4-bromophenyl)-1H-pyrazol-1-yl)pyridine (600 mg, 2.0 mmol), PdCl₂(PPh₃)₂ (70.2 mg, 5 mol %), CuI (19.0 mg, 5 mol %) in TEA (10 mL) and 1,4-dioxane (5.0 mL) was bubbled with dry N₂ for 5 minutes. Then ethynyltrimethylsilane (600 mg, 6.0 mmol) was added and this reaction mixture was refluxed at 100° C. till the complete consumption of the SM as determined by TLC and LC-MS. Then water was added and the mixture was extracted with EA three times, the combined organic layers were washed with brine and dried (anhydrous Na₂SO₄). After filtration and concentration, the residue was purified by flash column chromatography on silica gel to give the desired product as a yellow solid (600 mg, 95% yield). LC/MS: m/z [M⁺+1]=318.

### Example 28D

### Preparation of 4-(5-(4-ethynylphenyl)-1H-pyrazol-1-yl)pyridine

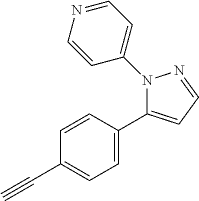 [SMILES: C#Cc1ccc(-c2ccnn2-c2ccncc2)cc1]

A mixture of 4-(5-(4-((trimethylsilyl)ethynyl)phenyl)-1H-pyrazol-1-yl)pyridine (600 mg, 2 mmol) in TBAF solution (6 mL, 1 M in THF). The reaction was stirred at room temperature till the complete consumption of the SM as determined by TLC and LC-MS. Then water was added and the mixture was extracted with EA three times, the combined organic layers were washed with brine and dried (anhydrous Na₂SO₄). After filtration and concentration, the residue was purified by flash column chromatography on silica gel to give the desired product as a light yellow solid (470 mg, 95% yield). LC/MS: m/z [M⁺+1]=246.

### Example 28

### Synthesis of 2-((4-(1-(pyridin-4-yl)-1H-pyrazol-5-yl)phenyl)ethynyl)quinoline

A mixture of 4-(5-(4-ethynylphenyl)-1H-pyrazol-1-yl)pyridine (98 mg, 0.4 mmol), PdCl₂(PPh₃)₂ (14.0 mg, 5 mol %), CuI (3.8 mg, 5 mol %) in TEA (4 mL) and 1,4-dioxane (2 mL) was bubbled with dry N₂ for 5 minutes. Then 2-bromoquinoline (82.4 mg, 0.4 mmol) was added and this reaction mixture was refluxed at 100° C. till the complete consumption of the SM as determined by TLC and LC-MS. Then water was added and the mixture was extracted with EA three times, the combined organic layers were washed with brine and dried (anhydrous Na₂SO₄). After filtration and concentration, the residue was purified by flash column chromatography on silica gel to give the desired product as a yellow solid (115.7 mg, 78% yield). LC/MS: m/z [M⁺+1]=373; HPLC retention time=2.81 minutes (Method A); ¹H NMR (400 M, CDCl₃) δ 8.59 (s, 2H), 8.18-8.12 (m, 2H), 7.85-7.73 (m, 3H), 7.69-7.50 (m, 4H), 7.31-7.25 (m, 4H), 6.58 (s, 1H).

### Example 29

### 2-[4-(3-Pyridin-4-yl-3H-imidazol-4-yl)-phenylethynyl]-quinoline

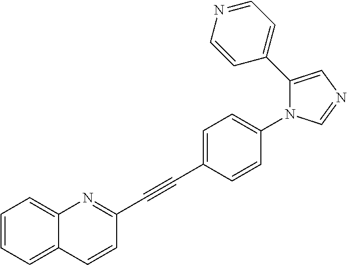 [SMILES: C(#Cc1ccc2ccccc2n1)c1ccc(-n2cncc2-c2ccncc2)cc1]

### Example 29A

### Preparation of 4-bromo-N-(pyridin-4-ylmethylene)aniline

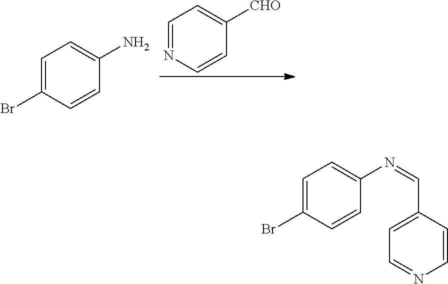

To a mixture of 4-bromoaniline (5 g, 29 mmol), isonicotinaldehyde (3 g, 29 mmol) in toluene (20 ml) was stirred at 110° C. overnight. Then the solution was concentrated and the residue was purified by column chromatography over silica gel using (PE:EA=4:1) to give 6 g of product as a solid (6 g, yield: 86%). MS (ESI) m/z=261/263 [M+H]⁺.

### Example 29B

### Preparation of 4-(1-(4-bromophenyl)-1H-imidazol-5-yl)pyridine

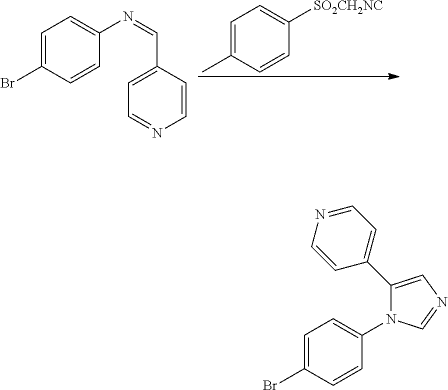

To a suspension of 4-bromo-N-(pyridin-4-ylmethylene)aniline (3 g, 11.5 mmol), (toluene-4-sulfonyl)-acetonitrile (3.37 g, 17.2 mmol), K₂CO₃ (3.2 g, 23 mmol) in DME/MeOH (40 ml/80 ml) was stirred at 120° C. overnight. The mixture was poured into water and extracted with EA. The organic layers were washed with brine, dried over Na₂SO₄ and concentrated. The residue was purified by column chromatography over silica gel using (PE:EA=1:1) to give the titled product as a yellow solid (2.7 g, yield: 79%); MS (ESI) m/z=300/302 [M+H]⁺.

### Example 29C

### Preparation of 4-(1-(4-((trimethylsilyl)ethynyl)phenyl)-1H-imidazol-5-yl)pyridine

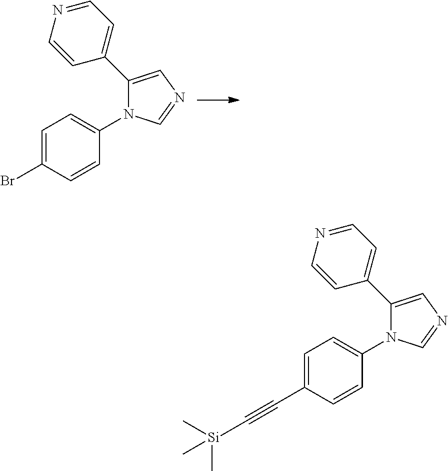 [SMILES: Brc1ccc(-n2cncc2-c2ccncc2)cc1 ; C[Si](C)(C)C#Cc1ccc(-n2cncc2-c2ccncc2)cc1]

A solution of 4-(1-(4-bromophenyl)-1H-imidazol-5-yl)pyridine (600 mg, 2 mmol), CuI (19 mg, 0.1 mmol), PdCl₂(PPh3)₂ (70 mg, 0.1 mmol) in TEA/dioxane (2:1) was bubbled with N₂ at room temperature for 5 minutes. To this, was added ethynyltrimethylsilane (0.86 ml, 6 mmol) and the resulting reaction mixture was again degassed for 2 minutes. Then the reaction mixture was heated at 100° C. for 1 hour under N₂. When LC/MS indicated the reaction was completed. The mixture was concentrated and purified by column chromatography over silica gel using (PE:EA=1:2) to give product as a white solid (500 mg, yield: 78%); MS (ESI) m/z=318 [M+H]⁺.

### Example 29D

### Preparation of 4-(1-(4-ethynylphenyl)-1H-imidazol-5-yl)pyridine

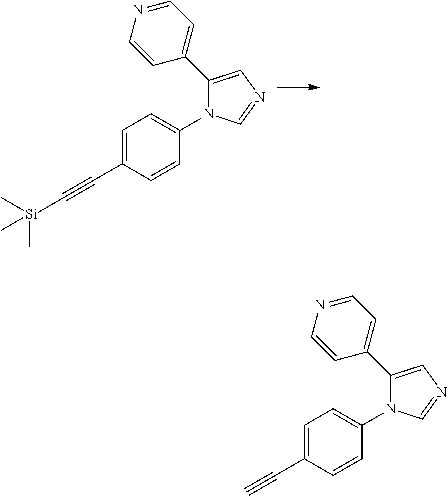 [SMILES: C[Si](C)(C)C#Cc1ccc(-n2cncc2-c2ccncc2)cc1 ; C#Cc1ccc(-n2cncc2-c2ccncc2)cc1]

To a solution of 4-(1-(4-((trimethylsilyl)ethynyl)phenyl)-1H-imidazol-5-yl)pyridine (200 mg, 0.63 mmol) in THF (3 ml), was added TBAF/THF (3 ml, 1M in THF) at 0° C. The reaction mixture was stirred at room temperature for 1 hour and the reaction was completed by LC/MS analysis. The solution was then diluted with EA and washed with water and brine consequently. The organic layers were dried over Na₂SO4 and concentrated. The residue was purified by column chromatography over silica gel using (PE:EA=1:2) to give the titled product as a white solid (140 mg, yield: 90%); MS (ESI) m/z=246 [M+H]⁺.

### Example 29

### Preparation of 2-[4-(3-Pyridin-4-yl-3H-imidazol-4-yl)-phenylethynyl]-quinoline

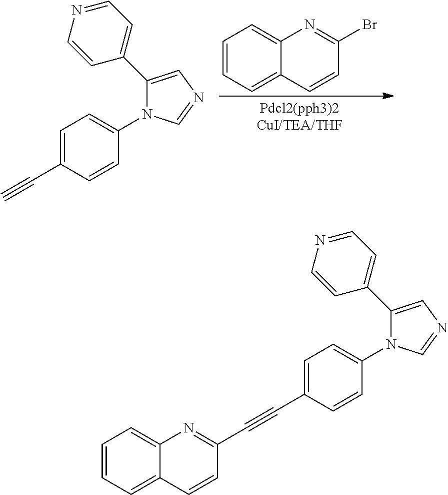

A solution of 2-Bromoquinazoline (131 mg, 0.63 mmol), CuI (5 mg, 0.026 mmol), PdCl₂(PPh3)₂ (20 mg, 0.028 mmol) in TEA/dioxane (2 ml) (2:1) was degassed with N₂ at room temperature for 5 minutes. To this was added 4-(1-(4-ethynylphenyl)-1H-imidazol-5-yl)pyridine (140 mg, 0.57 mmol) and the resulting reaction mixture was again degassed for 2 minutes. Then the reaction mixture was heated at 100° C. for 1 h under N₂. When LC/MS indicated the reaction was completed. The mixture was concentrated and purified by column chromatography over silica gel using (PE:EA=1:2) to give 40 mg of product as a white solid (40 mg, yield: 20%); ¹H NMR (400 MHz, DMSO) δ 8.51-8.46 (m, 3H), 8.16 (s, 1H), 8.03-8.04 (d, J=8.4 Hz, 2H), 7.82-7.86 (m, 3H), 7.77-7.79 (d, J=8.4 Hz, 1H), 7.66-7.70 (m, 1H), 7.60 (s, 1H), 7.44-7.47 (d, J=8.8 Hz, 2H), 7.13-7.14 (d, J=6 Hz, 2H); MS (ESI) m/z=373 [M+H]⁺; HPLC retention time=2.05 minutes (Method A).

### Example 30

### 2-((5-(1-ethyl-4-(pyridin-4-yl)-1H-pyrazol-3-yl)pyridin-2-yl)ethynyl)quinoline

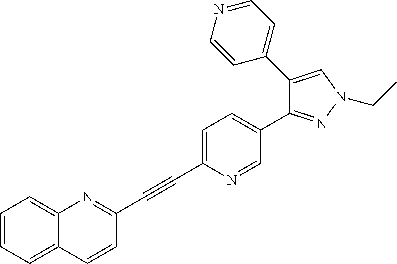 [SMILES: CCn1cc(-c2ccncc2)c(-c2ccc(C#Cc3ccc4ccccc4n3)nc2)n1]

### Example 30A

### Preparation of 6-bromo-N-methoxy-N-methylnicotinamide

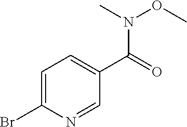

To a mixture of 6-bromonicotinic acid (3.0 g, 15 mmol), N,O-dimethyl hydroxylamine hydrochloride (1.75 g, 18 mmol) and Et₃N (2.60 mL, 18 mmol) in dry CH₂Cl₂ (40 mL) at 0° C., was added EDCI (3.45 g, 18 mmol) in portions. The mixture was then stirred at 25° C. for 2 hours and washed with water (40 mL). The organic layer was separated and the aqueous layer was extracted with CH₂Cl₂ (40 mL). The combined organic layers were washed with brine (20 mL), dried over MgSO₄, filtered and concentrated to give the product (3 g, yield: 83%) as a yellow oil which was used directly for the next step without further purification, LC/MS: m/z M⁺+1=245.

### Example 30B

### Preparation of 1-(6-bromopyridin-3-yl)-2-(pyridin-4-yl)ethanone

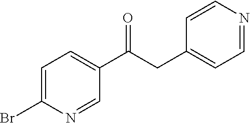 [SMILES: O=C(Cc1ccncc1)c1ccc(Br)nc1]

To a solution of 4-picoline (5.7 g, 61.5 mmol) in dry THF (35 mL) at 0° C., was added LDA (2.0 M in THF, 30.8 mL, 61.5 mmol) dropwise. After the addition, the mixture was stirred at 0° C. for 30 minutes. In a separated round bottom flask, 6-bromo-N-methoxy-N-methylnicotinamide (3 g, 12.3 mmol) was dissolved in dry THF (35 mL) and cooled to −60° C. Then 1.5 eq. of the 4-picoline anion was added dropwise. After 45 minutes, the remaining 4-picoline anion was added dropwise. The mixture was stirred at −60° C. for 2 hours and warmed to 28° C. slowly. After 16 hours, the reaction was quenched by addition of NH₄Cl (aq., 50 mL). Then water (15 mL) and ethyl acetate (50 mL) were added. The organic layer was separated and the aqueous layer was extracted with ethyl acetate (30 mL). The combined organic layers were washed with brine (30 mL), dried over Na₂SO₄, filtered, and concentrated. The residue was recrystallized from PE:EA (10:1) to give product as a yellow solid (2.5 g, yield: 73.5%); MS (ESI) m/z=277 [M+H]⁺.

### Example 30C

### Preparation of 2-bromo-5-(4-(pyridin-4-yl)-1H-pyrazol-3-yl)pyridine

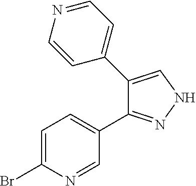 [SMILES: Brc1ccc(-c2n[nH]cc2-c2ccncc2)cn1]

1-(6-bromopyridin-3-yl)-2-(pyridin-4-yl)ethanone (2.2 g, 7.9 mmol) was heated at reflux in DMF-DMA (10 ml) for 1 hour and concentrated. The residue was dissolved in MeOH and Hydrazine hydrate in water (80%) (0.4 ml) was added. Then the reaction mixture was heated at 65° C. for 1 hour. When LC/MS indicated the reaction was completed. The mixture was concentrated and purified by column chromatography over silica gel using (PE:EA=10:1-1:1) to give the titled product (1.8 g, yield: 75.9%); MS (ESI) m/z=301 [M+H]⁺.

### Example 30D

### Preparation of

### 5-(4-(pyridin-4-yl)-1H-pyrazol-3-yl)-2-((trimethylsilyl)ethynyl)pyridine

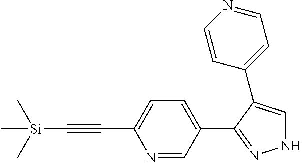 [SMILES: C[Si](C)(C)C#Cc1ccc(-c2n[nH]cc2-c2ccncc2)cn1]

To a mixture of 2-bromo-5-(4-(pyridin-4-yl)-1H-pyrazol-3-yl)pyridine (1.8 g, 6 mmol), PdCl₂(PPh₃)₂ (210 mg, 0.03 mmol) and CuI (57 mg, 0.03 mmol) in Et₃N/dioxane (20 mL), was bubbled with N₂ for 15 minutes. Then ethynyltrimethylsilane (1.76 g, 18 mmol) was added and the resulting mixture was stirred at 100° C. for 1 hour under N₂ protection. When LC/MS indicated the reaction was completed. The mixture was concentrated and purified by column chromatography over silica gel using (PE:EA=1:1) to give product as a white solid (1.9 g, yield: 94%); MS (ESI) m/z=333 [M+H]⁺.

### Example 30E

### Preparation of 2-ethynyl-5-(4-(pyridin-4-yl)-1H-pyrazol-3-yl)pyridine

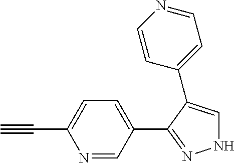 [SMILES: C#Cc1ccc(-c2n[nH]cc2-c2ccncc2)cn1]

5-(1-methyl-4-(pyridin-4-yl)-1H-pyrazol-3-yl)-2-((trimethylsilyl)ethynyl)pyridine (1.9 g, 6 mmol) was dissolved in TBAF-THF (20 mL). The mixture was stirred at 25° C. for 1 hour. Then the mixture was partitioned between water (20 mL) and ethyl acetate (20 mL). The organic layer was separated and the aqueous layer was extracted with ethyl acetate (2×10 mL). The combined organic layers were washed with brine (15 mL), dried over Na₂SO₄, filtered, and concentrated to give the crude product which was purified by column chromatography (PE:EA=1:1) to give the product as a white solid (1.0 g, yield: 71.4%); MS (ESI) m/z=247 [M+H]⁺.

### Example 30

### Preparation of

### 2-((5-(4-(pyridin-4-yl)-1H-pyrazol-3-yl)pyridin-2-yl)ethynyl)quinoline

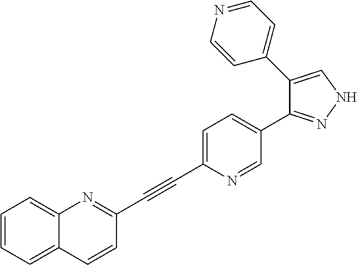 [SMILES: C(#Cc1ccc2ccccc2n1)c1ccc(-c2n[nH]cc2-c2ccncc2)cn1]

A mixture of 2-bromoquinoline (400 mg, 1.6 mmol), PdCl₂(PPh₃)₂ (56 mg, 0.008 mmol) and CuI (9.5 mg, 0.08 mmol) in Et₃N/dioxane (10 mL) was bubbled N₂ for 15 minutes. Then the crude mixture from the former step dissolved in 1,4-dioxane (4 mL), was added and the resulting mixture was bubbled to N₂ for 10 minutes. Then the resulting mixture was stirred at 100° C. for 1 hour under N₂ protection. When LC/MS indicated the reaction was completed. The mixture was concentrated and purified by column chromatography over silica gel using (PE: EA=1:1) to give the product as a white solid (600 mg, yield: 90%); MS (ESI) m/z=374 [M+H]⁺.

### Example 31

### 2-((5-(1-ethyl-4-(pyridin-4-yl)-1H-pyrazol-3-yl)pyridin-2-yl)ethynyl)quinoline

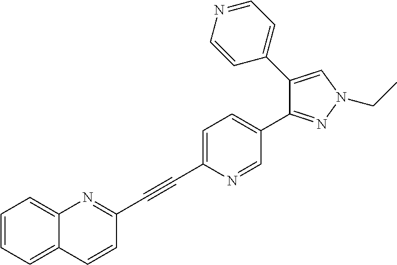 [SMILES: CCn1cc(-c2ccncc2)c(-c2ccc(C#Cc3ccc4ccccc4n3)nc2)n1]

To a suspension of 245-(4-(pyridin-4-yl)-1H-pyrazol-3-yl)pyridin-2-yl)ethynyl)quinoline (70 mg, 0.19 mmol) and Cs₂CO₃ (185 mg, 0.57 mmol) in DMF (1 ml), was added bromoethane (61 mg, 0.57 mmol) at room temperature. The mixture was stirred at room temperature for 15 minutes, and then diluted with EA, and washed with brine. The organic layers were then dried over Na₂SO₄, filtered, and concentrated. The residue was purified by prep-TLC to give the product as a yellow solid (30 mg, yield: 40%); ¹H NMR (400 MHz, CDCl₃) δ 8.82 (brs, 1H), 8.58 (brs, 2H), 8.19-8.21 (m, 3H), 7.59-7.89 (m, 7H), 7.22 (s, 2H), 4.31 (t, 2H), 1.61-1.65 (m, 3H); MS (ESI) m/z=402 [M+H]⁺; HPLC retention time=2.00 min (Method A).

### Example 32

### 2-((5-(1-(2-fluoroethyl)-4-(pyridin-4-yl)-1H-pyrazol-3-yl)pyridin-2-yl)ethynyl)quinoline

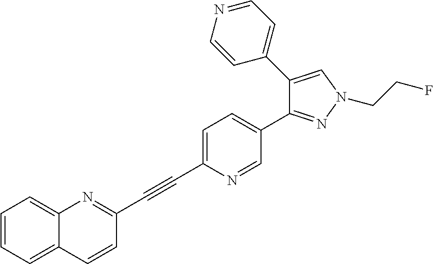 [SMILES: FCCn1cc(-c2ccncc2)c(-c2ccc(C#Cc3ccc4ccccc4n3)nc2)n1]

The title compound was synthesized using the procedures described in Example 31 to give product as a white solid (40 mg, yield: 50%); MS (ESI) m/z=420 [M+H]⁺; HPLC retention time=1.95 minutes (Method A); ¹H NMR (400 MHz, CDCl₃) δ 8.82 (s, 1H), 8.58 (brs, 2H), 8.15-8.21 (q, 2H), 7.87-7.89 (m, 2H), 7.72-7.79 (m, 4H), 7.58-7.62 (m, 1H), 7.21 (brs, 2H), 4.94-4.96 (t, 1H), 4.82-4.85 (t, 1H), 4.57-4.59 (t, 1H), 4.50-4.53 (t, 1H).

### Example 33

### 2-((4-(4-(4-methoxyphenyl)-1-methyl-1H-pyrazol-3-yl)phenyl)ethynyl)quinoline

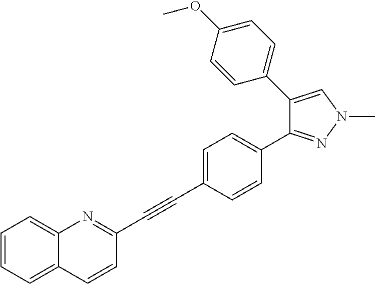 [SMILES: COc1ccc(-c2cn(C)nc2-c2ccc(C#Cc3ccc4ccccc4n3)cc2)cc1]

### Example 33A

### Preparation of N-methoxy-2-(4-methoxyphenyl)-N-methylacetamide

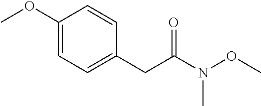

To a mixture of 2-(4-methoxyphenyl)acetic acid (30 g, 180.7 mmol), N,O-dimethyl hydroxylamine hydrochloride (21.2 g, 217 mmol) and Et₃N (22 g, 217 mmol) in dry CH₂Cl₂ (40 mL), was added EDCI (41.6 g, 217 mmol) in portions at 0° C. The mixture was then stirred at 25° C. for 2 hours and then washed with water (400 mL). The organic layer was separated and the aqueous layer was extracted with CH₂Cl₂ (400 mL). The combined organic layers were washed with brine (200 mL), dried over MgSO₄, filtered, and concentrated to give the product (37.9 g, yield: 90%) as yellow oil which was used directly for the next step without further purification; LC/MS: m/z M⁺1=210.

### Example 33B

### Preparation of 1-(4-bromophenyl)-2-(4-methoxyphenyl)ethanone

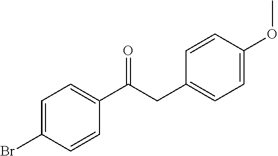 [SMILES: COc1ccc(CC(=O)c2ccc(Br)cc2)cc1]

To a solution of 1,4-dibromobenzene (22.6 g, 95.7 mmol) in dry THF (100 mL) at −65° C., was added n-BuLi (1.6 M in THF, 60 mL, 95.7 mmol) dropwise. After the addition, the mixture was stirred at −65° C. for 30 minutes. Then N-methoxy-2-(4-methoxyphenyl)-N-methylacetamide (16 g, 76.6 mmol) in THF (50 ml) was added dropwise. The mixture was allowed to warm to room temperature slowly. The reaction was quenched by addition of NH₄Cl (aq., 50 mL). Then water (150 mL) and ethyl acetate (150 mL) were added. The organic layer was separated and the aqueous layer was extracted with ethyl acetate (100 mL). The combined organic layers were washed with brine (100 mL), dried over Na₂SO₄, filtered, and concentrated. The residue was recrystallized from (PE:EA=10:1) to give product as a white solid (16 g, yield: 69.6%); MS (ESI) m/z=305 [M+H]⁺.

### Example 33C

### Preparation of 3-(4-bromophenyl)-4-(4-methoxyphenyl)-1-methyl-1H-pyrazole

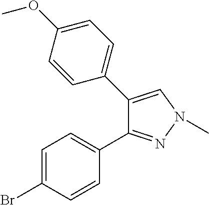

1-(4-bromophenyl)-2-(4-methoxyphenyl)ethanone (10 g, 32.8 mmol) was heated at reflux in DMF-DMA (30 ml) for 1 hour and concentrated. The residue was dissolved in MeOH and methylhydrazine in water (40%) (8 ml) was added. Then the reaction mixture was heated at 65° C. for 1 hour. When LC/MS indicated the reaction was completed. The mixture was concentrated and purified by column chromatography over silica gel using (PE:EA=10:1-1:1) to give the product (7.5 g, yield: 67%); MS (ESI) m/z=343 [M+H]⁺.

### Example 33D

### Preparation of 4-4-methoxyphenyl)-1-methyl-3-(4-trimethylsilyl)ethynyl)phenyl)-1H-pyrazole

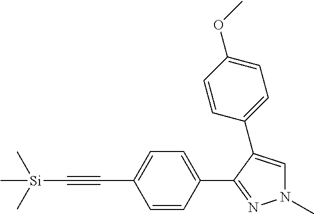

A mixture of 3-(4-bromophenyl)-4-(4-methoxyphenyl)-1-methyl-1H-pyrazole (7 g, 20.5 mmol), PdCl₂(PPh₃)₂ (702 mg, 1 mmol) and CuI (190 mg, 1 mmol) in Et₃N/dioxane (50 mL) was bubbled N₂ for 15 minutes. Then ethynyltrimethylsilane (6 g, 61.4 mmol) was added and the resulting mixture was stirred at 100° C. for 1 hour under N₂ protection. When LC/MS indicated the reaction was completed, the mixture was concentrated and purified by column chromatography over silica gel using (PE:EA=5:1) to give the product as a white solid (6 g, yield: 81%); MS (ESI) m/z=361 [M+H]⁺.

### Example 33E

### Preparation of 3-(4-ethynylphenyl)-4-(4-methoxyphenyl)-1-methyl-1H-pyrazole

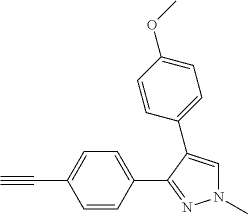 [SMILES: C#Cc1ccc(-c2nn(C)cc2-c2ccc(OC)cc2)cc1]

4-(4-methoxyphenyl)-1-methyl-3-(4-((trimethylsilyl)ethynyl)phenyl)-1H-pyrazole (3 g, 8.3 mmol) was dissolved in TBAF-THF (8.3 mL). The mixture was stirred at 25° C. for 1 hour. Then the mixture was partitioned between water (50 mL) and ethyl acetate (50 mL). The organic layer was separated and the aqueous layer was extracted with ethyl acetate (2×50 mL). The combined organic layers were washed with brine (55 mL), dried over Na₂SO₄, filtered, and concentrated to give the crude product which was purified by column chromatography (PE:EA=15:1) to give the product as a white solid (2.4 g, yield: 90%); MS (ESI) m/z=289 [M+H]⁺.

### Example 33

### Preparation of 2-((4-(4-(4-methoxyphenyl)-1-methyl-1H-pyrazol-3-yl)phenyl)ethynyl)quinoline

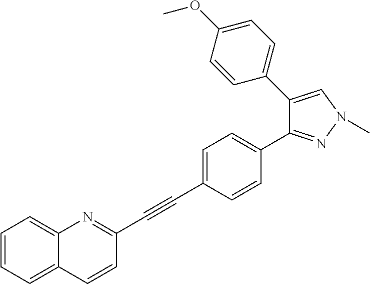 [SMILES: COc1ccc(-c2cn(C)nc2-c2ccc(C#Cc3ccc4ccccc4n3)cc2)cc1]

A mixture of 2-bromoquinoline (1.73 g, 8.3 mmol), PdCl₂(PPh₃)₂ (291 mg, 0.415 mmol) and CuI (79 mg, 0.415 mmol) in Et₃N/dioxane (30 mL) was bubbled N₂ for 15 minutes. Then the crude mixture (2.4 g, 8.3 mmol) from the former step dissolved in 10 mL of 1,4-dioxane was added and the resulting mixture was bubbled to N₂ for 10 minutes. Then the mixture was stirred at 100° C. for 1 h under N₂ protection. When LC/MS indicated the reaction was completed, the mixture was concentrated and purified by column chromatography over silica gel using (PE:EA=5:1) to give the product as a yellow solid (2.4 g, yield: 70.6%); MS (ESI) m/z=388 [M+H]⁺; HPLC retention time=3.41 minutes (Method A); ¹H NMR (400 MHz, CD₃OD) δ 8.14-8.18 (t, 2H), 7.83 (m, 1H), 7.74-7.78 (m, 1H), 7.55-7.63 (m, 6H), 7.21-7.23 (d, J=8 Hz, 2H), 6.89-6.91 (d, J=8 Hz, 2H), 4.00 (s, 3H), 3.85 (s, 3H).

### Example 34

### 4-(1-methyl-3-(4-(quinolin-2-ylethynyl)phenyl)-1H-pyrazol-4-yl)phenol

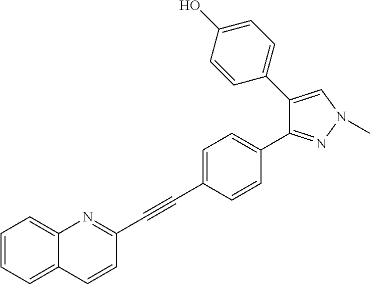 [SMILES: Cn1cc(-c2ccc(O)cc2)c(-c2ccc(C#Cc3ccc4ccccc4n3)cc2)n1]

BBr₃ (1.81 mL, 14.46 mmol) was slowly added to a solution of 2-((4-(4-(4-methoxyphenyl)-1-methyl-1H-pyrazol-3-yl)phenyl)ethynyl)quinoline (2 g, 4.82 mmol) in DCM (60 mL) at 0° C. The mixture was stirred at room temperature for 3 hours. Ice water was added and resulting mixture was neutralized by satd. NaHCO₃ aq solution. DCM was added and organic phase was isolated and then washed with brine, dried over Na₂SO₄, filtered, and concentrated. The mixture was purified by column chromatography (silica gel; DCM:MeOH=30:1) to give the titled product (330 mg, yield: 17%); ¹H NMR (400 MHz, DMSO): δ 9.49 (s, 1H), 8.45 (d, J=8.4 Hz, 1H), 8.03 (d, J=8 Hz, 2H), 7.82 (m, 2H), 7.61-7.67 (m, 3H), 7.53 (d, J=8.4 Hz, 2H), 7.07 (d, J=8.4 Hz, 2H), 6.77 (d, J=8.4 Hz, 2H), 3.91 (s, 3H); MS (ESI) m/z=402[M+H]⁺; HPLC retention time=3.01 minutes (Method A).

### Example 35

### 2-((4-(3-(pyridin-4-yl)-1H-pyrazol-4-yl)phenyl)ethynyl)quinoline

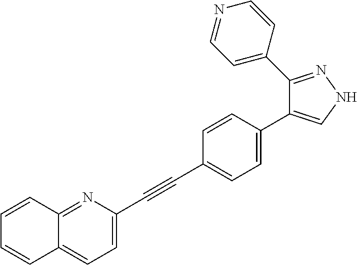 [SMILES: C(#Cc1ccc2ccccc2n1)c1ccc(-c2c[nH]nc2-c2ccncc2)cc1]

### Example 35A

### Preparation of 2-(4-bromophenyl)-3-oxo-3-(pyridin-4-yl)propanenitrile

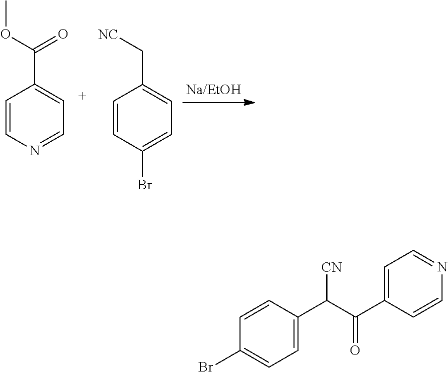

To a stirred mixture of Na (2 g, 14.6 mmol) in EtOH (20 ml) at room temperature, was added a mixture of 2-(4-bromophenyl)acetonitrile (4.27 g, 21.9 mmol) and methyl isonicotinate (2 g, 14.6 mmol) under N₂ over a period of 15 minutes. The mixture was stirred at reflux for 2 hours cooled to room temperature and diluted with water. The aqueous solution was extracted with ether and the aqueous solution extracted was neutracted with aqueous 1N HCl. The yellow precipitate thus obtained was collected by filtration, washed with water, and dried to give the product as a yellow solid (2.3 g, yield: 50%); MS (ESI) m/z=301/303 [M+H]⁺.

### Example 35B

### Preparation of 2-(4-bromophenyl)-1-(pyridin-4-yl)ethanone

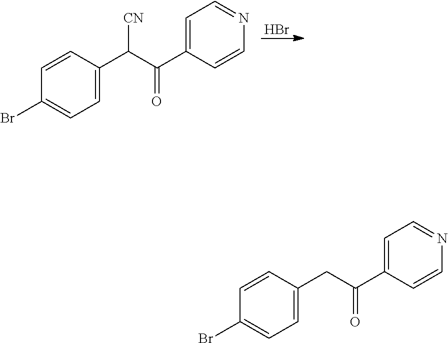 [SMILES: N#CC(C(=O)c1ccncc1)c1ccc(Br)cc1 ; Br ; O=C(Cc1ccc(Br)cc1)c1ccncc1]

2-(4-bromophenyl)-3-oxo-3-(pyridin-4-yl)propanenitrile was dissolved in 48% aqueous HBr (60 ml) and the resulting mixture was stirred at reflux overnight. The reaction mixture was cooled to room temperature and neutralized with aqueous K₂CO₃. The mixture was extracted with EtOAc. The combine organic extracts were washed with brine, dried over MgSO₄, and concentrated to afford a crude product and purified by column chromatography over silica gel using (PE:EA=4:1) to give the product as a yellow solid (650 mg, yield: 43.8%); MS (ESI) m/z=276/278 [M+H]⁺.

### Example 35C

### Preparation of 4-(4-(4-bromophenyl)-1H-pyrazol-3-yl)pyridine

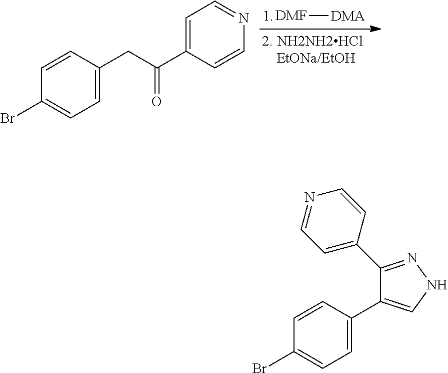

A mixture of 2-(4-bromophenyl)-1-(pyridin-4-yl)ethanone (400 mg, 1.45 mmol) in DMF-DMA (3 ml) was heated at reflux for 1 hour and concentrated. The residue was dissolved in MeOH (2 ml) and methylhydrazine in water (40%, 3 ml) was added. Then the reaction mixture was heated at 65° C. for 1 hour. When LC/MS indicated the reaction was completed, the mixture was concentrated and purified by column chromatography over silica gel using (PE:EA=4:1-2:1) to give the product as a white solid (320 mg, yield: 73.8%); MS (ESI) m/z=300/302[M+H]⁺.

### Example 35D

### Preparation of 4-(4-(4-((trimethylsilyl)ethynyl)phenyl)-1H-pyrazol-3-yl)pyridine

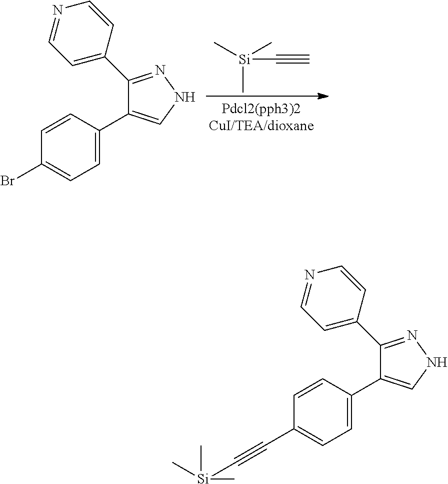

A solution of 4-(4-(4-bromophenyl)-1H-pyrazol-3-yl)pyridine (320 g, 1.07 mmol), CuI (8.4 mg, 0.044 mmol), PdCl₂(PPh3)₂ (30.9 mg, 0.044 mmol) in TEA/dioxane (2:1) was bubbled with N₂ at room temperature for 5 minutes. To this, was added ethynyltrimethylsilane (0.314 g, 3.21 mmol) and the resulting reaction mixture was again degassed for 2 minutes. Then the reaction mixture was heated at 100° C. for 1 hour under N₂. When LC/MS indicated the reaction was completed, the mixture was concentrated and purified by column chromatography over silica gel using (PE:EA=2:1) to give the product as a yellow solid (270 mg, yield: 80%); MS (ESI) m/z=318 [M+H]⁺.

### Example 35D

### Preparation of 4-(4-(4-ethynylphenyl)-1H-pyrazol-3-yl)pyridine

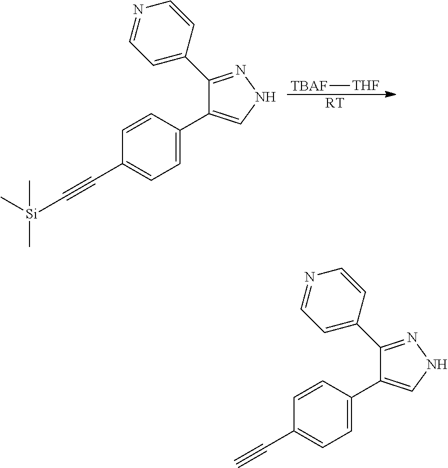

To a solution of 4-(4-(4-((trimethylsilyl)ethynyl)phenyl)-1H-pyrazol-3-yl)pyridine in THF (1 ml) at 0° C., was added TBAF/THF (1 ml, 1 M in THF). The reaction mixture was stirred at room temperature for 1 hour. When LC/MS indicated the reaction was completed, the solution was then diluted with EA and washed with water and brine consecutively. The organic layers were dried over Na₂CO₃ and concentrated. The residue was purified by column chromatography over silica gel using (PE:EA=2:1) to give the product as a yellow solid (176 mg, yield: 84.5%); MS (ESI) m/z=246 [M+H]⁺.

### Example 35

### Preparation of 2-((4-(3-(pyridin-4-yl)-1H-pyrazol-4-yl)phenyl)ethynyl)quinoline

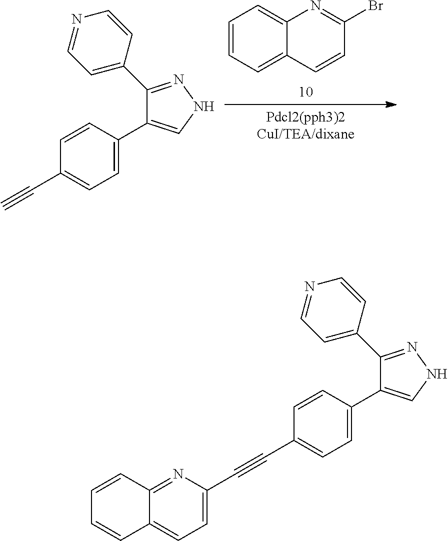 [SMILES: C#Cc1ccc(-c2c[nH]nc2-c2ccncc2)cc1 ; Brc1ccc2ccccc2n1 ; C(#Cc1ccc2ccccc2n1)c1ccc(-c2c[nH]nc2-c2ccncc2)cc1]

A solution of 2-bromoquinoline (160 mg, 0.77 mmol), CuI (5.29 mg, 0.0278 mmol), PdCl₂(PPh₃)₂ (19.48 mg, 0.028 mmol) in TEA/dioxane (3 ml) (2:1) was degassed with N₂ at room temperature for 5 minutes. To this, was added 4-(4-(4-ethynylphenyl)-1H-pyrazol-3-yl)pyridine (170 mg, 0.69 mmol) and the resulting reaction mixture was again degassed for 2 minutes. Then the reaction mixture was heated at 100° C. for 1 hour under N₂. When LC/MS indicated the reaction was completed, the mixture was concentrated and purified by column chromatography over silica gel using (PE:EA=1:1) to give the product as a yellow solid (135 mg, yield: 52.3%); ¹H NMR (400 MHz, CDCl₃) δ 8.62 (brs, 2H), 8.18 (t, J=8.8 Hz, 2H), 7.75-7.86 (m, 3H), 7.66 (dd, J=5.2 Hz, 3H), 7.59 (t, J=6.8 Hz, 2H), 7.47 (brs, 2H), 7.34 (d, J=8 Hz, 2H); MS (ESI) m/z=373 [M+H]⁺; HPLC retention time=2.31 minutes (Method A).

### Example 36

### 2-((4-(1-methyl-3-(pyridin-4-yl)-1H-pyrazol-4-yl)phenyl)ethynyl)quinoline

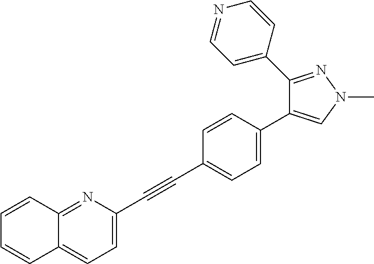 [SMILES: Cn1cc(-c2ccc(C#Cc3ccc4ccccc4n3)cc2)c(-c2ccncc2)n1]

To stirred mixture of 2-((4-(3-(pyridin-4-yl)-1H-pyrazol-4-yl)phenyl)ethynyl) quinoline (50 mg, 0.134 mmol) in 1 ml DMF was added a mixture of MeI (57.2 mg, 0.402 mmol) And Cs₂CO₃ (130.65 mg, 0.402 mmol) at 0° C. temperature over a period of 10 minutes and diluted with water. The aqueous solution was extracted with EtOAc. The combine organic extracts were washed with saturated aqueous sodium chloride, dried over MgSO₄ and concentrated to obtain a crude product and purified by prep-TLC ((DCM: MeOH=20:1) to give Example 35 as a yellow solid (26 mg, yield: 43.8%); ¹H NMR (400 MHz, CDCl₃): δ 8.18 (d, J=8.8 Hz, 2H), 7.84 (d, J=8 Hz, 1H), 7.78 (t, 1H), 7.53-7.66 (m, 6H), 7.31 (m, 2H), 4.02 (s, 3H); MS (ESI) m/z=387 [M+H]⁺; HPLC retention time=2.28 minutes (Method A).

### Example 37

### 2-((4-(1-methyl-5-(pyridin-4-yl)-1H-pyrazol-4-yl)phenyl)ethynyl)quinoline

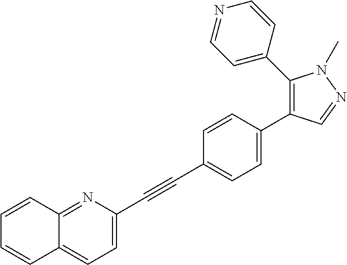 [SMILES: Cn1ncc(-c2ccc(C#Cc3ccc4ccccc4n3)cc2)c1-c1ccncc1]

Example 37 was isolated as an isomer during preparation of Example 36 as a yellow solid (12 mg); ¹H NMR (400 MHz, CDCl₃): δ 8.76 (brs, 2H), 8.17 (t, J=8.4 Hz, 2H), 7.78-7.84 (m, 3H), 7.55-7.62 (m, 4H), 7.28 (brs, 2H), 7.18 (d, J=8 Hz, 2H), 3.87 (S, 3H); MS (ESI) m/z=387 [M+H]⁺; HPLC retention time=2.74 minutes (Method A).

### Example 38

### 4-(1-methyl-3-(4-(quinolin-2-ylethynyl)phenyl)-1H-pyrazol-4-yl)morpholine

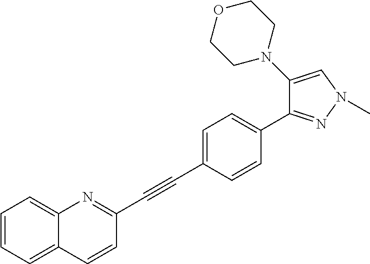 [SMILES: Cn1cc(N2CCOCC2)c(-c2ccc(C#Cc3ccc4ccccc4n3)cc2)n1]

### Example 38A

### Preparation of 1-(4-bromophenyl)-2-morpholinoethanone

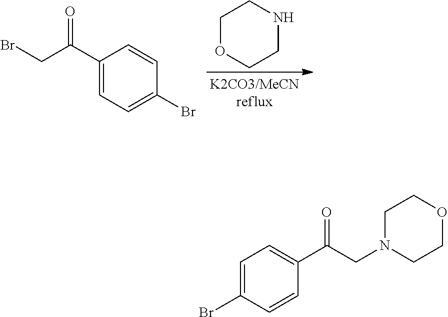 [SMILES: O=C(CBr)c1ccc(Br)cc1 ; C1COCCN1 ; O=C(CN1CCOCC1)c1ccc(Br)cc1]

To a stirred mixture of morpholine (0.95 g, 10.87 mmol) in MeCN (40 ml), were added a mixture of K₂CO₃ (3 g, 21.7 mmol) and 2-bromo-1-(4-bromophenyl)ethanone (2 g, 7.25 mmol) at room temperature. The mixture was stirred at reflux for 1 hour, cooled to room temperature and diluted with water. The aqueous solution was extracted with ether. The yellow solid was collected by filtration to give the titled product (790 mg, yield: 38.5%); MS (ESI) m/z=284/286 [M+H]⁺.

### Example 38B

### Preparation of 4-(3-(4-bromophenyl)-1-methyl-1H-pyrazol-4-yl)morpholine

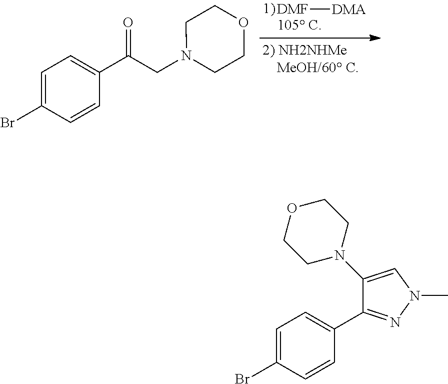

The title compound was synthesized using the procedures described in Example 1 to give the product as a yellow solid (480 mg, yield: 53.6%); MS (ESI) m/z=322/324 [M+H]⁺

### Example 38C

### Preparation of 4-(1-methyl-3-(4-((trimethylsilyl)ethynyl)phenyl)-1H-pyrazol-4-yl)morpholine

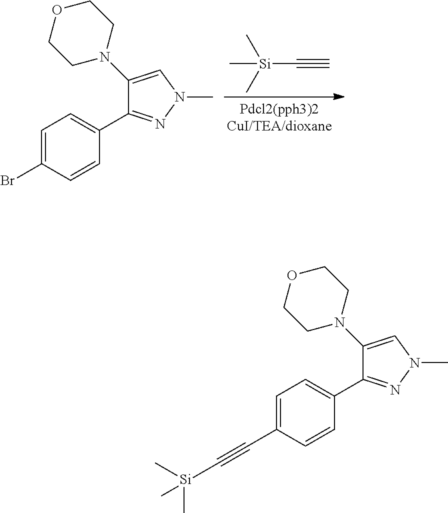

The title compound was synthesized using the procedures described in Example 1 to give the product as a yellow solid (90 mg, yield: 17%); MS (ESI) m/z=340 [M+H]⁺

### Example 38D

### Preparation of 4-(3-(4-ethynylphenyl)-1-methyl-1H-pyrazol-4-yl)morpholine

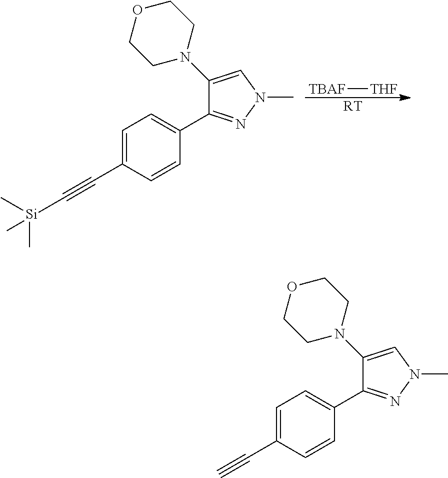

The title compound was synthesized using the procedures described in Example 1 to give the product as a yellow solid (480 mg, yield: 97.5%); MS (ESI) m/z=268 [M+H]⁺.

### Example 38

### Preparation of 4-(3-(4-bromophenyl)-1-methyl-1H-pyrazol-4-yl)morpholine

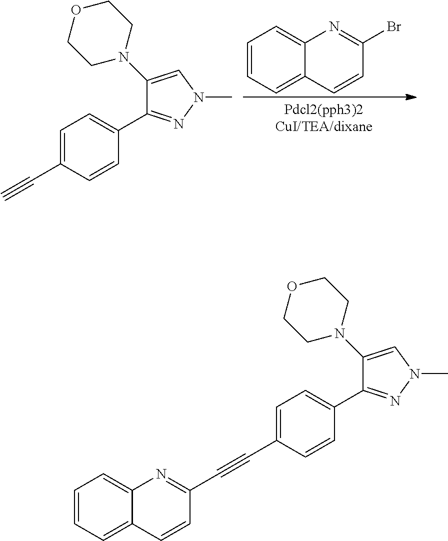

The title compound was synthesized using the procedures described in Example 1 to give the titled product as a yellow solid (50 mg, yield: 53.6%); ¹H NMR (400 MHz, CDCl₃) δ 7.90-7.93 (m, 2H), 7.51-7.54 (m, 2H), 7.15 (s, 1H), 3.82 (t, 4H), 3.82 (t, 4H); MS (ESI) m/z=395[M+H]⁺; HPLC retention time=3.21 min (Method A).

### Biological Testing:

### PDE10A Inhibition Assay:

Full-length human PDE10A cDNA is amplified using PCR and cloned into pcDNA3.0 vector (Invitrogen). After verified by DNA sequencing, the plasmid is transfected into HEK293 cell using the Lipofectamine 2000 reagent (Invitrogen). Cells are subsequently harvested and sonicated in cell lysis buffer. After centrifuged at 10,000 g for 20 minutes, supernatant containing cytosolic hPDE10A enzyme is collected and stored at −80° C. PDE10A inhibition assay is performed using the IMAP fluorescence polarization assay (Molecular Devices) according to the manufacturer's protocols. A fixed amount of the hPDE10A enzyme is pretreated with the test compounds, following the addition of fluorescein-labeled cAMP/cGMP. After 1 hour incubation, the IMAP binding reagent is added and incubated for another hour. The fluorescence polarization is then quantified with a fluorescence polarization reader Envision from Perkin Elmer. IC₅₀ values are calculated using the Prism program (GraphPad Software).

### Conditioned Avoidance Response:

Conditioned avoidance response (CAR) assay is performed in male CD-1 mice using commercially available shuttle boxes (Coulbourn Instruments), which are divided into two equal-sized compartments by an arch style door and enclosed in sound-attenuating chambers. The metal grid floors equipped with a constant current shock generator are put into the shuttle boxes. Training consists of 30 cycles with cue light, sound and opening of the door for 10 s as conditioned stimulus, followed by a 0.8 mA shock of unconditioned stimulus, which is terminated when the animal crosses to the other side of the shuttle box or after 10 s. The intertrial interval is 15 s with the door remaining closed. Training is continued until animals show a stable avoidance performance (at least 80%). Compounds are administered as indicated before test sessions. All training and testing procedures are controlled and recorded by a computer program. ED₅₀ values are determined using the Prism program (GraphPad Software).

The following are biological data obtained for compounds of Examples 1-37 using the biological assays described hereinabove. A compound with the value “++++” had an IC₅₀ value less than or equal to 10 nM; a compound with the value “+++” had an IC₅₀ value between 10 nM and 100 nM; a compound with the value “++” had an IC₅₀ value greater than or equal to 100 nM but less than 1,000 nM; a compound with the value “+” had an IC₅₀ value greater than or equal to 1,000 nM.

| Example No. | IC₅₀ Value |
|---|---|
| 1 | ++++ |
| 2 | ++++ |
| 3 | ++++ |
| 4 | ++++ |
| 5 | +++ |
| 6 | ++++ |
| 7 | ++++ |
| 8 | ++ |
| 9 | ++ |
| 10 | ++++ |
| 11 | ++++ |
| 12 | ++++ |
| 13 | ++++ |
| 14 | ++++ |
| 15 | ++++ |
| 16 | ++++ |
| 17 | ++++ |
| 18 | ++++ |
| 19 | ++++ |
| 20 | +++ |
| 21 | +++ |
| 22 | ++++ |
| 23 | +++ |
| 24 | ++++ |
| 25 | ++++ |
| 26 | +++ |
| 27 | ++++ |
| 28 | ++++ |
| 29 | ++++ |
| 31 | ++++ |
| 32 | ++++ |
| 33 | +++ |
| 34 | +++ |
| 35 | ++++ |
| 36 | ++++ |
| 37 | +++ |

What is claimed is:

1. A compound having the following Formulae, or a pharmaceutical acceptable salt thereof: 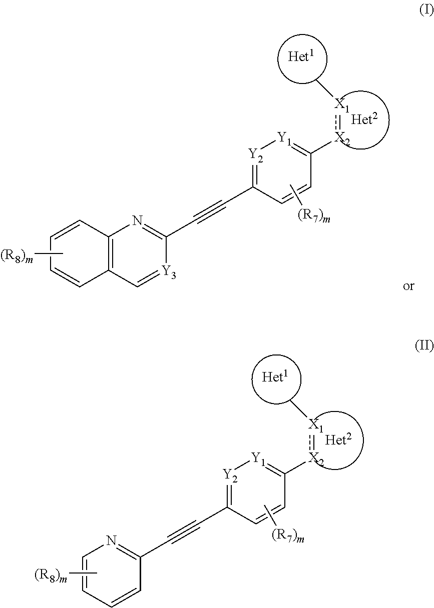
wherein X₁ and X₂ are each independently C or N;
Y₁, Y₂ and Y₃ are each independently C or N;
each Het¹ is independently aryl or 3-7-membered heterocycle containing at least one heteroatom selected from the group consisting of nitrogen, oxygen, and sulfur, in which said aryl may be optionally substituted by one to three R₁ and one R₂, and said heterocycle may be optionally substituted by one to three R₁;
each Het² is independently 3-7-membered heterocycle containing at least one heteroatom selected from the group consisting of nitrogen, oxygen, and sulfur, in which said heterocycle may be optionally substituted by one R₅ and one to three R₆;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₂ is independently H, OH, CN, or NRₐR_{b};
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

2. The compound of claim 1, wherein each Het¹ is independently selected from Formulae A1-A5 below: 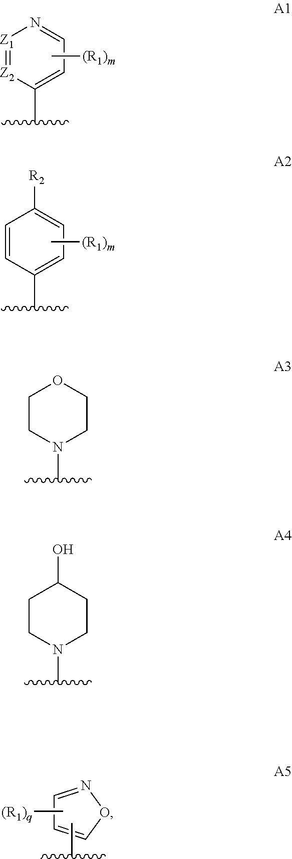
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₂ is independently H, OH, CN, or NRₐR_{b};
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
m is 1-3; and
q is 1-2.

3. The compound of claim 1, wherein each Het² is independently selected from Formulae B1-B23 below: 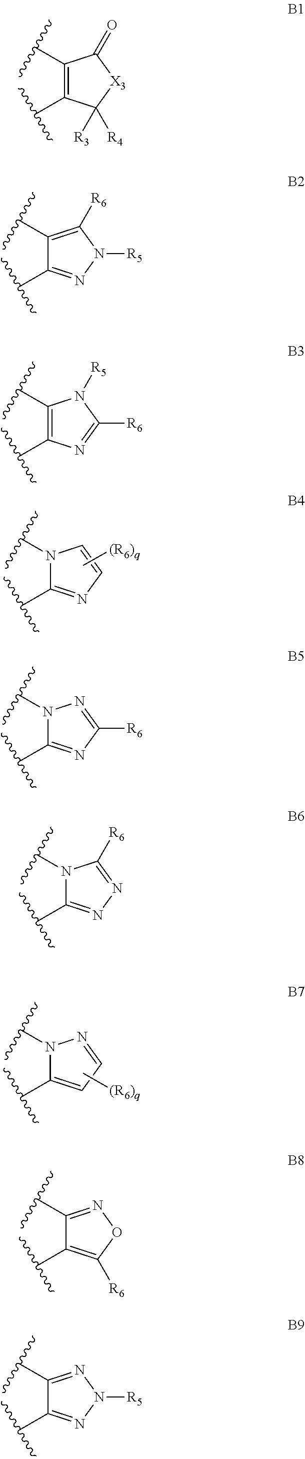 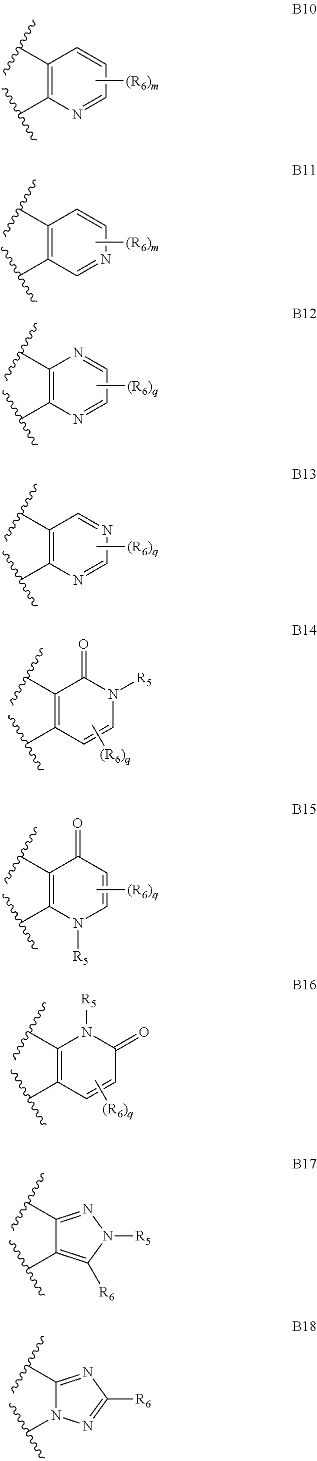 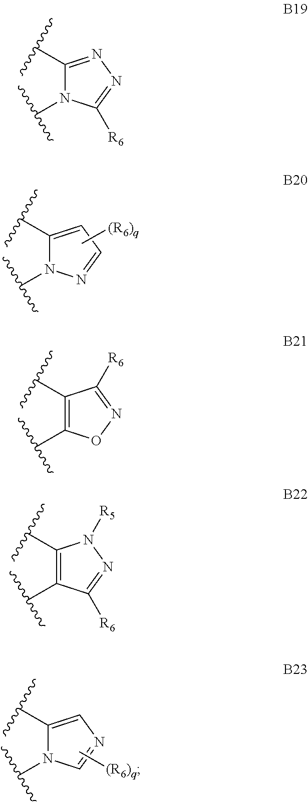
wherein X₃ is O, or NR₅;
each R₃ and R₄ is independently H, or (C₁-C₆)alkyl;
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
m is 1-3; and
q is 1-2.

4. The compound of claim 1, having the structure of Formula (III): 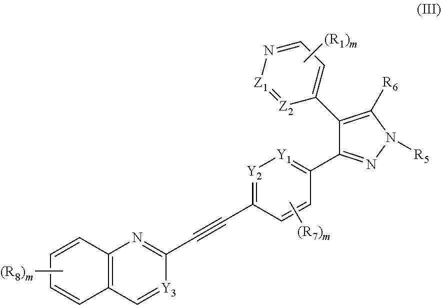
wherein Y₁, Y₂ and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

5. The compound of claim 1, having the structure of Formula (IV): 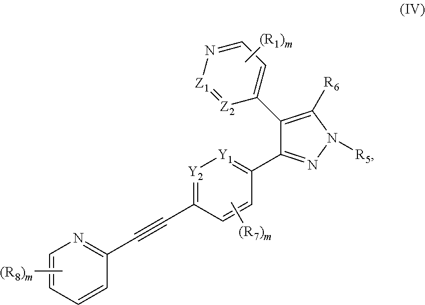
wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

6. The compound of claim 1, having the structure of Formula (V): 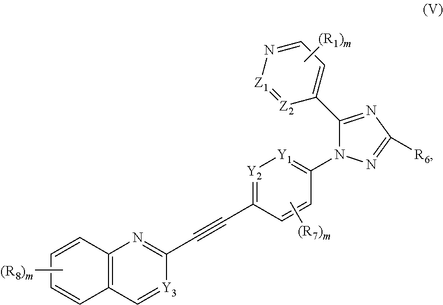
wherein Y₁, Y₂ and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

7. The compound of claim 1, having the structure of Formula (VI): 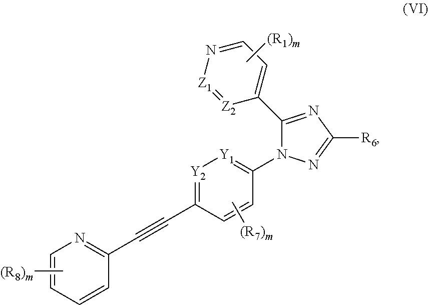
wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

8. The compound of claim 1, having the structure of Formula (VII): 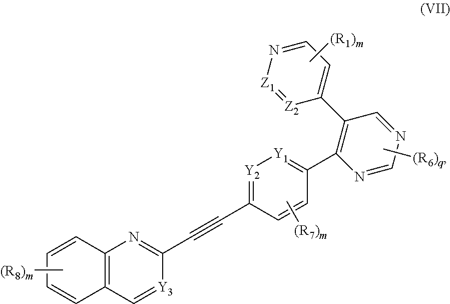
wherein Y₁, Y₂ and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6;
m is 1-3; and
q is 1-2.

9. The compound of claim 1, having the structure of Formula (VIII): 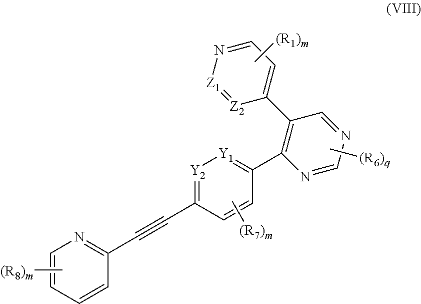
wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

10. The compound of claim 1, having the structure of Formula (IX): 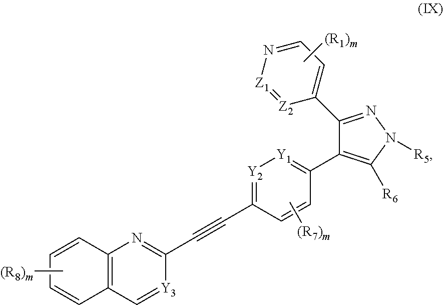
wherein Y₁, Y₂, and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
m is 1-3.

11. The compound of claim 1, having the structure of Formula (X): 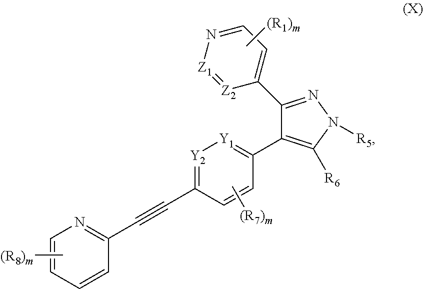
wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

12. The compound of claim 1, having the structure of Formula (XI): 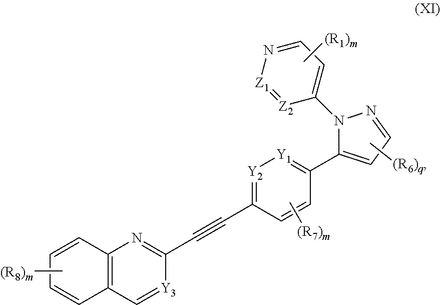
wherein Y₁, Y₂, and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
q is 1-2; and m is 1-3.

13. The compound of claim 1, having the structure of Formula (XII): 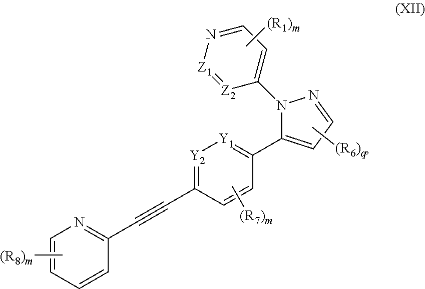
wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

14. The compound of claim 1, having the structure of Formula (XIII): 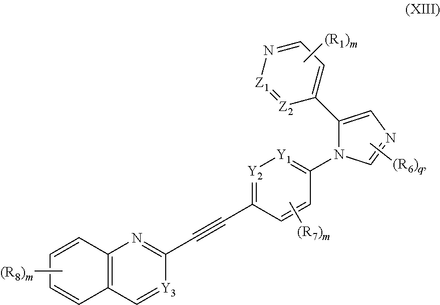
wherein Y₁, Y₂, and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
q is 1-2; and m is 1-3.

15. The compound of claim 1, having the structure of Formula (XIV): 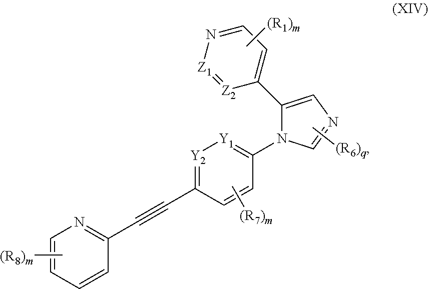
wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

16. The compound of claim 1, having the structure of Formula (XV): 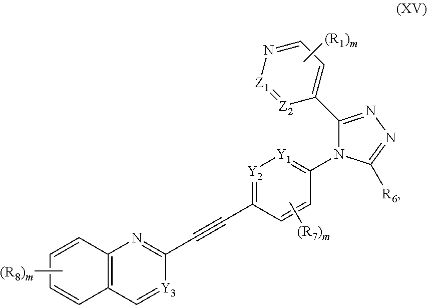
wherein Y₁, Y₂, and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
q is 1-2; and m is 1-3.

17. The compound of claim 1, having the structure of Formula (XVI): 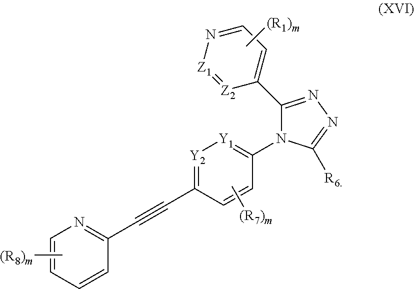
wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

18. The compound of claim 1, having the structure of Formula (XVII): 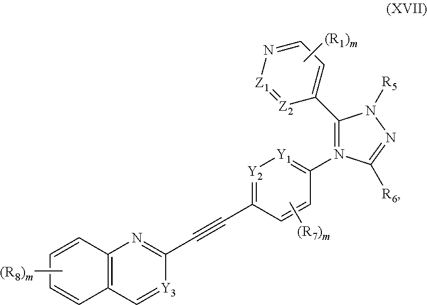
wherein Y₁, Y₂, and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
q is 1-2; and m is 1-3.

19. The compound of claim 1, having the structure of Formula (XVIII): 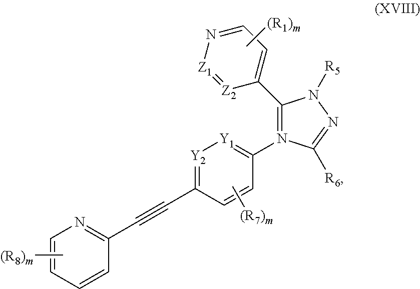
wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₅ is independently H, (C₁-C₄)alkyl, (C₃-C₇)cycloalkyl, (C₁-C₆)haloalkyl (e.g., CF₃), (CH₂)ₙORₐ, or (CH₂)ₙNRₐR_{b};
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

20. The compound of claim 1, having the structure of Formula (IXX): 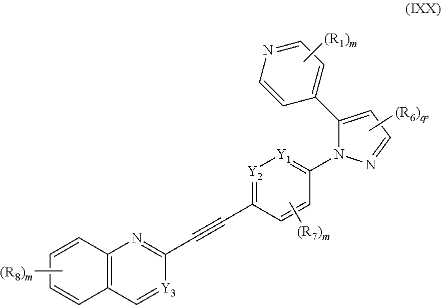
wherein Y₁, Y₂, and Y₃ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
q is 1-2; and m is 1-3.

21. The compound of claim 1, having the structure of Formula (XX): 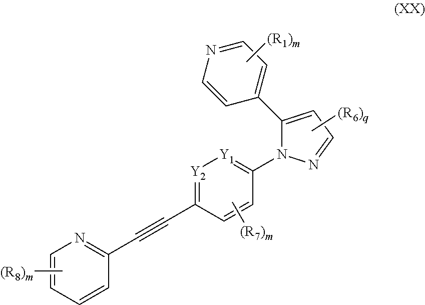
wherein Y₁ and Y₂ are each independently C or N;
Z₁ and Z₂ are each independently C or N;
each R₁ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, or haloalkyl (e.g., CF₃);
each R₆ is independently H, halogen, OH, CN, (C₁-C₆)alkyl, (C₂-C₆)alkenyl, (C₂-C₆)alkynyl, (C₁-C₆)alkoxy, (C₃-C₇)cycloalkyl, (C₁-C₆)alkylthio, NRₐR_{b}, (C₁-C₆)haloalkyl (e.g., CF₃), (CH₂)ₙORₐ, (CH₂)ₙSRₐ, (CH₂)ₙNRₐR_{b}, (CH₂)ₙ(C₁-C₆)haloalkyl;
R₇ and R₈ are each independently H, halogen, OH, CN, OCF₃, (C₁-C₆)alkyl, (C₂-C₆)alkenyl, (C₂-C₆)alkynyl, (C₁-C₆)alkoxy, (C₃-C₇)cycloalkyl, 3-7-membered heterocycle, (C₁-C₆)alkylthio, NRₐR_{b}, (C₁-C₆)haloalkyl (e.g., CF₃), (CH₂)ₙ(C₃-C₇)cycloalkyl, (CH₂)ₙORₐ, (CH₂)ₙSRₐ, (CH₂)ₙNRₐR_{b}, or (CH₂)ₙ(C₁-C₆)haloalkyl, in which said heterocycle contains at least one heteroatom selected from the group consisting of nitrogen, oxygen and sulfur and may be optionally substituted by from one to three groups which may be the same or different selected from the group consisting of halogen, OH, CN, (C₁-C₄)alkyl, (C₁-C₄)haloalkyl (e.g., CF₃) and (C₁-C₄)alkoxy;
each occurrence of Rₐ and R_{b} is independently hydrogen or (C₁-C₆)alkyl, or Rₐ and R_{b}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to three groups which may be the same or different selected from the group consisting of (C₁-C₄)alkyl, phenyl and benzyl;
n is 1-6; and
m is 1-3.

22. A pharmaceutical composition comprising at least one compound of claim 1 and a pharmaceutically-acceptable carrier or diluent.

23. A method for treating a psychotic disorder, an anxiety disorder, or a neurodegenerative disorder in a mammalian species in need thereof, the method comprising administering to the mammalian species a therapeutically effective amount of at least one compound according to claim 1.
